(19) United States
(12) Patent Application Publication
(10) Pub. No.: US 2025/0270650 A1
(43) Pub. Date: Aug. 28, 2025

(54) METHODS FOR IDENTIFYING GENOMIC REGIONS OF LOW BACKGROUND METHYLATION

(71) Applicant: Flagship Pioneering Innovations VI, LLC, Cambridge, MA (US)

(72) Inventor: Jocelyn Charlton, London (GB)

(21) Appl. No.: 18/977,707

(22) Filed: Dec. 11, 2024

Related U.S. Application Data
(60) Provisional application No. 63/609,140, filed on Dec. 12, 2023.

(51) Int. Cl. C12Q 1/6886

(52) U.S. Cl. CPC C12Q 1/6886; C12Q 2600/154

(57) ABSTRACT

Low background regions (LBRs) refer to genomic regions comprising one or more CpG sites that are differentially methylated in cancer and non-cancer samples. Disclose methods involve identifying LBRs comprising one or more CpG sites whose methylation statuses sufficiently distinguish between samples that contain a presence of a cancer signature and samples that do not contain a presence of a cancer signature. LBRs enable improved detection of presence or absence of cancer signatures while using smaller samples and fewer consumable reagents.

### CROSS-REFERENCE TO RELATED APPLICATIONS

This application claims the benefit of and priority to U.S. Provisional Patent Application No. 63/609,140 filed Dec. 12, 2023, the entire disclosure of which is hereby incorporated by reference in its entirety for all purposes.

### SEQUENCE LISTING

The instant application contains a Sequence Listing which has been submitted electronically in XML format and is hereby incorporated by reference in its entirety. Said Sequence Listing, created on Dec. 8, 2024, is named FLG-020WO_SL and is 178,319 bytes in size.

### BACKGROUND

Cancer diagnostic methods have traditionally relied on the detection of somatic mutations, often in the form of single nucleotide variants (SNVs). However, SNVs are generally low-recurring or non-recurring across various cancers (either same cancers or different cancers), thereby rendering the use of SNVs as predictors of cancer challenging. Furthermore, as SNVs are often present at low levels, their use as cancer predictors often requires reference biopsy samples. Therefore, new methods of detecting cancer signatures with improved predictability are needed.

### SUMMARY

Disclosed herein are methods, non-transitory computer readable media, systems, and kits for identifying and using low background regions (LBRs) for predicting presence or absence of a cancer signature in a sample obtained from a subject. LBRs refer to genomic regions comprising one or more CpG sites that are differentially methylated in cancer and non-cancer samples. LBRs have low signal in non-cancer samples, thereby enabling identification of fragments originating from cancer above the noise. Thus, distinguishing between samples that contain a presence of a cancer signature and samples that do not contain a presence of a cancer signature can involve analyzing methylation statuses of one or more CpG sites of LBRs.

The advantages of using the LBRs for predicting presence or absence of a cancer signature are several-fold:
Improved detection of presence or absence of a cancer signature within a sample that is less than a threshold volume (e.g., less than 20 mL). This eliminates the need to obtain large samples from individuals, which may not be possible for all individuals.
Improved detection of presence or absence of a cancer signature within a sample using fewer than a threshold number of CpG sites (e.g., fewer than 500 CpG sites) within the low LBRs. By having to analyze only a limited number of CpG sites, this reduces the consumption of reagents that are needed when performing assays to obtain methylation statuses of the CpG sites.

Disclosed herein is a method for detecting a cancer signature in a sample, the method comprising: obtaining nucleic acids from a single sample of less than 20 mL obtained from a subject; determining methylation statuses of a plurality of CpG sites in the nucleic acids, wherein the plurality of CpG sites comprise at least a subset of CpG sites within a plurality of low background regions (LBRs) identified for exhibiting a maximum methylation frequency of the one or more CpG sites between 0 and 5% across non-cancer samples and/or a minimum methylation frequency of the one or more CpG sites between 5% and 30% across cancer samples, detecting presence or absence of the cancer signature in the sample according to the determined methylation statuses of the plurality of CpG sites, wherein the detection of presence or absence of the cancer signature in the sample achieves at least 40% sensitivity at a given specificity of at least 85%.

In various embodiments, the single sample obtained from the subject is less than 10 mL. In various embodiments, the single sample obtained from the subject is less than 5 mL. In various embodiments, the maximum methylation frequency is between 0 and 3%. In various embodiments, the maximum methylation frequency is about 2%. In various embodiments, the minimum methylation frequency is between 15% and 25%. In various embodiments, the minimum methylation frequency is between 18% and 22%. In various embodiments, the minimum methylation frequency is about 20%. In various embodiments, the plurality of CpG sites comprises fewer than 500 CpG sites. In various embodiments, the plurality of CpG sites comprises fewer than 250 CpG sites. In various embodiments, the plurality of CpG sites comprises fewer than 100 CpG sites. In various embodiments, the plurality of CpG sites consist of the subset of CpG sites within the plurality of LBRs. In various embodiments, the plurality of CpG sites consist of all CpG sites within the plurality of LBRs. In various embodiments, a quantity of the subset of CpG sites within the plurality of LBRs for which methylation statuses are determined is inversely related to a volume of the sample.

In various embodiments, determining methylation statuses of the plurality of CpG sites in the nucleic acids comprises performing one or more assays, wherein an assay comprises one or more of: a. sequencing nucleic acids via targeted sequencing, whole genome sequencing, or whole genome bisulfite sequencing; b. a nucleic acid amplification assay; c. a target enrichment assay; and d. an assay that generates methylation information. In various embodiments, the nucleic acid amplification assay is a PCR assay. In various embodiments, the PCR assay comprises a real-time PCR assay, quantitative real-time PCR (qPCR) assay, digital PCR (dPCR) assay, allele-specific PCR assay, or reverse-transcription PCR assay. In various embodiments, determining methylation statuses of a plurality of CpG sites in the nucleic acids comprises performing the target enrichment assay. In various embodiments, the target enrichment assay comprises hybrid capture. In various embodiments, performing the one or more assays comprise: obtaining bisulfite converted nucleic acids; and selectively amplifying target regions of the bisulfite converted nucleic acids. In various embodiments, the target regions comprise the plurality of CpG sites.

In various embodiments, performing one or more assays comprises providing one or more probes that bind to the subset of CpG sites within the plurality of low background regions (LBRs). In various embodiments, at least one probe binds to between 1 and 100 CpG sites within a LBR. In various embodiments, at least ten probes bind to at least 1 CpG site and less than 100 CpG sites of the plurality of LBRs. In various embodiments, at least one probe binds to between 10% and 90% of CpG sites within a LBR. In various embodiments, at least one probe binds to between 20% and 80%, between 30% and 70%, or between 40% and 60% of CpG sites within a LBR. In various embodiments, at least one probe binds to every CpG site within a LBR. In various embodiments, the plurality of LBRs are selected from LBRs identified in Table 1 (e.g., any of SEQ ID NOs: 1-130).

Additionally disclosed herein is a method for detecting a cancer signature in a sample, the method comprising: obtaining nucleic acids from a sample obtained from a subject; determining methylation statuses of a plurality of CpG sites in the nucleic acids, the plurality of CpG sites comprising fewer than 500 CpG sites, wherein the fewer than 500 CpG sites comprise at least a subset of CpG sites within a plurality of low background regions (LBRs) identified for exhibiting according to either a maximum methylation frequency of the one or more CpG sites across non-cancer samples and/or a minimum methylation frequency of the one or more CpG sites across cancer samples, detecting presence or absence of the cancer signature in the sample according to the determined methylation statuses of the plurality of CpG sites comprising fewer than 500 CpG sites, wherein the detection of presence or absence of the cancer signature in the sample according to the determined methylation statuses of the plurality of CpG sites comprising fewer than 500 CpG sites achieves at least 40% sensitivity at a given specificity of at least 85%. In various embodiments, the sample comprises a single sample of less than 20 mL. In various embodiments, the single sample obtained from the subject is less than 10 mL. In various embodiments, the single sample obtained from the subject is less than 5 mL. In various embodiments, the maximum methylation frequency of the one or more CpG sites is between 0 and 5% across non-cancer samples. In various embodiments, the maximum methylation frequency is between 0 and 3%. In various embodiments, the maximum methylation frequency is about 2%. In various embodiments, the minimum methylation frequency of the one or more CpG sites is between 5% and 30% across cancer samples. In various embodiments, the minimum methylation frequency is between 15% and 25%. In various embodiments, the minimum methylation frequency is between 18% and 22%. In various embodiments, the minimum methylation frequency is about 20%. In various embodiments, the plurality of CpG sites comprises fewer than 250 CpG sites. In various embodiments, the plurality of CpG sites comprises fewer than 100 CpG sites. In various embodiments, the plurality of CpG sites consist of the subset of CpG sites within the plurality of LBRs. In various embodiments, the plurality of CpG sites consist of all CpG sites within the plurality of LBRs. In various embodiments, a quantity of the subset of CpG sites within the plurality of LBRs for which methylation statuses are determined is inversely related to a volume of the sample.

In various embodiments, determining methylation statuses of the plurality of CpG sites in the nucleic acids comprises performing one or more assays, wherein an assay comprises one or more of: a. sequencing nucleic acids via targeted sequencing, whole genome sequencing, or whole genome bisulfite sequencing; b. a nucleic acid amplification assay; c. a target enrichment assay; and d. an assay that generates methylation information. In various embodiments, the nucleic acid amplification assay is a PCR assay. In various embodiments, the PCR assay comprises a real-time PCR assay, quantitative real-time PCR (qPCR) assay, digital PCR (dPCR) assay, allele-specific PCR assay, or reverse-transcription PCR assay. In various embodiments, determining methylation statuses of a plurality of CpG sites in the nucleic acids comprises performing the target enrichment assay. In various embodiments, the target enrichment assay comprises hybrid capture. In various embodiments, performing the one or more assays comprise: obtaining bisulfite converted nucleic acids; and selectively amplifying target regions of the bisulfite converted nucleic acids. In various embodiments, the target regions comprise the plurality of CpG sites. In various embodiments, performing one or more assays comprises providing one or more probes that bind to the subset of CpG sites within the plurality of low background regions (LBRs). In various embodiments, at least one probe binds to between 1 and 100 CpG sites within a LBR. In various embodiments, at least ten probes bind to at least 1 CpG site and less than 100 CpG sites of the plurality of LBRs. In various embodiments, at least one probe binds to between 10% and 90% of CpG sites within a LBR. In various embodiments, at least one probe binds to between 20% and 80%, between 30% and 70%, or between 40% and 60% of CpG sites within a LBR. In various embodiments, at least one probe binds to every CpG site within a LBR. In various embodiments, the plurality of LBRs are selected from LBRs identified in Table 1 (e.g., any of SEQ ID NOs: 1-130).

Additionally disclosed herein is a method for identifying a low background region (LBR), the method comprising: obtaining a first set of methylation statuses of a plurality of CpG sites of nucleic acids from a first plurality of samples; obtaining a second set of methylation statuses of a plurality of CpG sites of nucleic acids from a second plurality of samples; determining the low background region comprising one or more CpG sites that are differentially methylated between the first and second sets of methylation statuses, wherein the determination of the low background region is according to one of: a maximum methylation frequency of one or more CpG sites of the first set of methylation statuses; a minimum methylation frequency of one or more CpG sites of the second set of methylation statuses; or a threshold differential methylation frequency of one or more CpG sites between the first set of methylation statuses and the second set of methylation statuses. In various embodiments, the first plurality of samples are non-cancer samples. In various embodiments, the second plurality of samples are cancer samples of a common cancer type. In various embodiments, the first plurality of samples are cancer samples of a cancer type that differs from a cancer type of the second plurality of samples. In various embodiments, the second plurality of samples comprise between 20 and 1000 samples, between 30 and 800 samples, between 40 and 600 samples, or between 50 and 500 samples. In various embodiments, the second plurality of samples comprise cancer biopsies or cell-free DNA. In various embodiments, the first plurality of samples comprise between 20 and 1000 samples, between 30 and 800 samples, between 40 and 600 samples, or between 50 and 500 samples.

In various embodiments, methods disclosed herein further comprise: ranking the LBR comprising the one or more CpG sites against one or more additional LBRs comprising one or more additional CpG sites that are differentially methylated between the first and second sets of methylation statuses. In various embodiments, methods disclosed herein further comprise: selecting the LBR according to its rank. In various embodiments, the LBR and the one or more additional LBRs are ranked according to differential methylation frequencies of one or more CpG sites of the LBR and one or more CpG sites of each of the one or more additional LBRs. In various embodiments, the LBR and the one or more additional LBRs are ranked according to their significance in distinguishing cancer and non-cancer samples. In various embodiments, the LBR and the one or more additional LBRs are ranked according to an importance analysis conducted by performing a machine learning model. In various embodiments, the machine learning model is a random forest model.

### BRIEF DESCRIPTION OF THE DRAWINGS

These and other features, aspects, and advantages of the present invention will become better understood with regard to the following description and accompanying drawings. It is noted that wherever practicable, similar or like reference numbers may be used in the figures and may indicate similar or like functionality. For example, a letter after a reference numeral, such as “sample 310A,” indicates that the text refers specifically to the element having that particular reference numeral. A reference numeral in the text without a following letter, such as “sample 310,” refers to any or all of the elements in the figures bearing that reference numeral (e.g., “sample 310” in the text refers to reference numerals “sample 310A” and/or “sample 310B” in the figures).

FIG. 1A an overall flow process for predicting presence or absence of a signature in a sample, in accordance with an embodiment.

FIG. 1B is a block diagram of the signature detection system for predicting presence or absence of a signature in a sample, in accordance with an embodiment.

FIG. 2 depicts an example conversion of nucleic acids, in accordance with an embodiment.

FIG. 3 is an example diagram for identifying a low background region, in accordance with an embodiment.

FIGS. 4A, 4B, and 4C are example flow charts for using CpG sites of low background regions and/or identifying low background regions, in accordance multiple embodiments.

FIG. 5 illustrates an example computer for implementing the entities shown in FIGS. 1A-1B, 2, 3, and 4A-4C.

FIG. 6 depicts differential methylation of 130 LBRs across cancer cfDNA and non-cancer cfDNA.

FIG. 7 shows that the 130 LBRs achieved at least 80% sensitivity across all tumor stages at a 90% target specificity.

### DETAILED DESCRIPTION

### Definitions

Terms used in the claims and specification are defined as set forth below unless otherwise specified.

The term “about” refers to a value that is within 10% above or below the value being described. For example, the term “about 5 nM” indicates a range of from 4.5 nM to 5.5 nM.

The terms “subject,” “patient,” and “individual” are used interchangeably and encompass a cell, tissue, or organism, human or non-human, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humor.

The term “CpG site” refers to a location of a genome that has cytosine and guanine separated by only one phosphate group, and is often denoted as “5′-C-phosphate-G-3”, or “CpG” for short. Regions with a high frequency of CpG sites are commonly referred to interchangeably as “CG islands,” “CpG islands,” or “CGIs”. In various embodiments, low background regions (LBRs), as described herein, can contain CGIs or portions thereof. Example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety.

The phrase “low background region” or “LBR” refer to genomic regions comprising one or more CpG sites that are differentially methylated in cancer and non-cancer samples. The phrase “differentially methylated” refers to a difference in methylation of one or more CpG sites between two types of samples. In particular embodiments, “differentially methylated” is used in reference to one or more CpG sites, where the one or more CpG sites are more frequently methylated in cancer samples and are more frequently non-methylated in non-cancer samples. For example, one or more CpG sites that are more frequently methylated in cancer samples may have a mean methylation frequency greater than a minimum methylation frequency threshold. As another example, one or more CpG sites that are more frequently non-methylated in cancer samples may have a mean methylation frequency less than a maximum methylation frequency threshold. Generally, the one or more differentially methylated CpG sites in LBRs enable differentiation of samples that have a presence of a cancer signature and samples that have an absence of a cancer signature. In various embodiments, LBRs are identified by comparing methylation statuses of one or more CpG sites of cancer and non-cancer samples to each other or to a comparison metric. For example, LBRs may be identified for satisfying a maximum methylation frequency of one or more CpG sites, satisfying a minimum methylation frequency of one or more CpG sites, satisfying a threshold differential methylation frequency, or combinations thereof.

The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

It must be noted that, as used in the specification, the singular forms “a,” “an” and “the” include plural referents unless the context clearly dictates otherwise.

### Overview

Disclosed herein are methods, non-transitory computer readable media, systems, and kits for predicting presence or absence of a cancer signature in a sample obtained from a subject using low background regions (LBRs). Generally, LBRs include genomic regions comprising one or more CpG sites that are differentially methylated in cancer and non-cancer. Thus, interrogation of methylation statuses of one or more CpG sites in the LBRs enables differentiation of samples that have a presence of a cancer signature and samples that have an absence of a cancer signature.

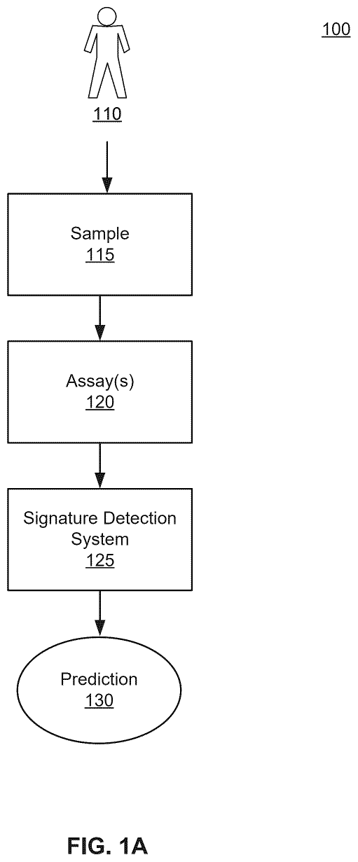

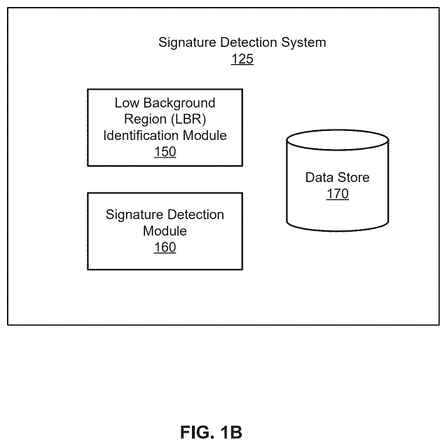

Reference is now made to FIG. 1A, which is an overall flow process for predicting presence or absence of a signature in a sample, in accordance with an embodiment. Although FIG. 1A shows the flow process in relation to a single subject 110, in various embodiments, the flow process can be performed for more than a single subject 110 (e.g., for thousands, millions, tens of millions, or hundreds of millions of subjects).

As shown in FIG. 1A, a step involves obtaining a sample 115 from the subject 110. In various embodiments, multiple samples 115 may be obtained from the subject 110 at multiple timepoints. In various embodiments, a sample is any of a blood sample, a plasma sample, a stool sample, a urine sample, a semen sample, an intraocular fluid sample, a peritoneal sample, a pleural fluid sample, an amniotic fluid sample, a cerebrospinal fluid sample, a mucous sample, or a saliva sample. In some embodiments, the biological sample may include a liquified biopsy obtained from a solid tissue and processed (e.g., through tissue trituration and/or enzymatic treatment) to produce a liquid sample. In particular embodiments, a sample obtained from the subject 110 is a blood sample, such as a liquid biopsy. The sample can be obtained by the subject or by a third party, e.g., a medical professional. Examples of medical professionals include physicians, emergency medical technicians, nurses, first responders, psychologists, phlebotomists, medical physics personnel, nurse practitioners, surgeons, dentists, and any other medical professional as would be known to one skilled in the art. In various embodiments, the sample 115 can be obtained from the subject 110 by a reference lab.

In various embodiments, multiple samples 115 may be obtained from the subject 110. For example, in some embodiments, two or more samples 115 are obtained from the subject 110. In some embodiments, three or more samples 115, four or more samples 115, five or more samples 115, six or more samples 115, seven or more samples 115, eight or more samples 115, nine or more samples 115, or ten or more samples 115 are obtained from the subject 110. In various embodiments, two samples 115 are obtained from the subject 110. In various embodiments, three samples 115 are obtained from the subject 110. In various embodiments, four samples 115 are obtained from the subject 110.

In various embodiments, the subject 110 may be a cancer subject and therefore, the sample 115 obtained from the subject 110 is a cancer sample. For example, the subject 110 may have been previously diagnosed with cancer and/or is not suspected of having cancer and therefore, is deemed a cancer subject. In various embodiments, the subject 110 may be a non-cancer subject and therefore, the sample 115 obtained from the subject 110 is a non-cancer sample. For example, the subject 110 may not have been previously diagnosed with cancer and therefore, is deemed a non-cancer subject. In various embodiments, the subject 110 may be a healthy subject and therefore, the sample 115 obtained from the subject 110 is a healthy sample. For example, the subject 110 is not suspected of having a disease or disorder and therefore, is deemed a healthy subject. As further discussed herein, cancer samples and non-cancer samples can be useful for identifying LBRs that distinguish between signatures in cancer samples and signatures in non-cancer samples.

In various embodiments, the sample 115 obtained from the subject 110 is of a limited volume. In various embodiments, the sample 115 is less than 1 L in volume. In various embodiments, the sample 115 is less than 500 mL in volume, less than 400 mL in volume, less than 300 mL in volume, less than 200 mL in volume, less than 100 mL in volume, less than 90 mL in volume, less than 80 mL in volume, less than 70 mL in volume, less than 60 mL in volume, less than 50 mL in volume, less than 40 mL in volume, less than 30 mL in volume, less than 20 mL in volume, less than 10 mL in volume, less than 9 mL in volume, less than 8 mL in volume, less than 7 mL in volume, less than 6 mL in volume, less than 5 mL in volume, less than 4 mL in volume, less than 3 mL in volume, less than 2 mL in volume, less than 1 mL in volume, less than 900 μL in volume, less than 800 μL in volume, less than 700 μL in volume, less than 600 μL in volume, less than 500 μL in volume, less than, 400 μL in volume, less than 300 μL in volume, less than 200 μL in volume, less than 100 μL in volume, less than 90 μL in volume, less than 80 μL in volume, less than 70 μL in volume, less than 60 μL in volume, less than 50 μL in volume, less than 30 μL in volume, less than 20 μL in volume, or less than 10 μL in volume. In particular embodiments, the sample 115 is less than 10 mL in volume.

In some embodiments, the sample 115 may include nucleic acids that are informative for predicting presence or absence of a cancer signature in the sample. In various embodiments, the nucleic acids include cell-free DNA (cfDNA). In various embodiments, the nucleic acids include cell-free DNA fragments. In various embodiments, the cfDNA can be derived from tumor cells and is referred to herein as circulating tumor DNA (ctDNA). In particular embodiments, the nucleic acids include cfDNA fragments across a plurality of genomic locations. In various embodiments, genomic locations can include one or more CpG sites whose methylation statuses may be informative for predicting presence or absence of a cancer signature. In various embodiments, genomic locations can be locations found in low background regions (LBRs), such as LBRs shown in Table 1. Such LBRs include one or more CpG sites whose methylation statuses are informative for predicting presence or absence of a cancer signature. Further details of exemplary LBRs, genomic locations, and CpG sites are described herein.

One or more assays 120 are performed to determine methylation statuses of a plurality of CpG sites in the nucleic acids. In various embodiments, the plurality of CpG sites comprise at least a subset of CpG sites within a plurality of low background regions (LBRs). In particular embodiments, the one or more assays 120 are performed to determine methylation statuses of a plurality of CpG sites, wherein all CpG sites are located within a plurality of LBRs, such as the LBRs identified in Table 1.

In various embodiments, performing one or more assays 120 involves converting nucleic acids in the sample 115 obtained from the subject 110. In various embodiments, converting the nucleic acid involves converting unmethylated nucleotides (e.g., cytosines) to another nucleotide (a “converted nucleotide”, as used herein). In various embodiments, methylated cytosines are protected from conversion (e.g., deamination) during the conversion step. This enables subsequent downstream differentiation of methylated cytosines and unmethylated cytosines. Further details of exemplary assays 120 are described herein.

The signature detection system 125 analyzes the methylation statuses of the plurality of CpG sites and predicts presence or absence of a cancer signature in the sample. In various embodiments, the signature detection system 125 can be embodied as a computer system and therefore, the steps performed by the signature detection system 125 are steps performed by a computer system. For example, the signature detection system 125 may implement a computational algorithm to analyze the methylation statuses of the plurality of CpG sites. In various embodiments, the signature detection system 125 deploys a trained machine learning model to analyze the methylation statuses of the plurality of CpG sites and to predict the presence or absence of a cancer signature in the sample.

The prediction 130 refers to a prediction generated by the signature detection system 125 that indicates whether a sample has a presence or absence of a cancer signature. In various embodiments, a presence of a cancer signature in a sample refers to a presence of tumor derived nucleic acids, such as tumor derived cell-free DNA, in the sample. In various embodiments, the prediction 130 additionally or alternatively refers to a tissue of origin prediction. For example, if the sample is predicted to have a presence of a cancer signature, the prediction 130 can additionally or alternatively identify the tissue from which the cancer signature originates.

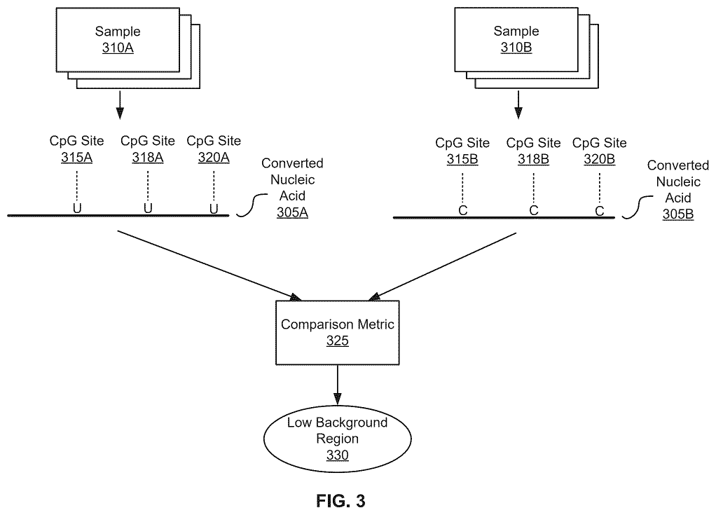

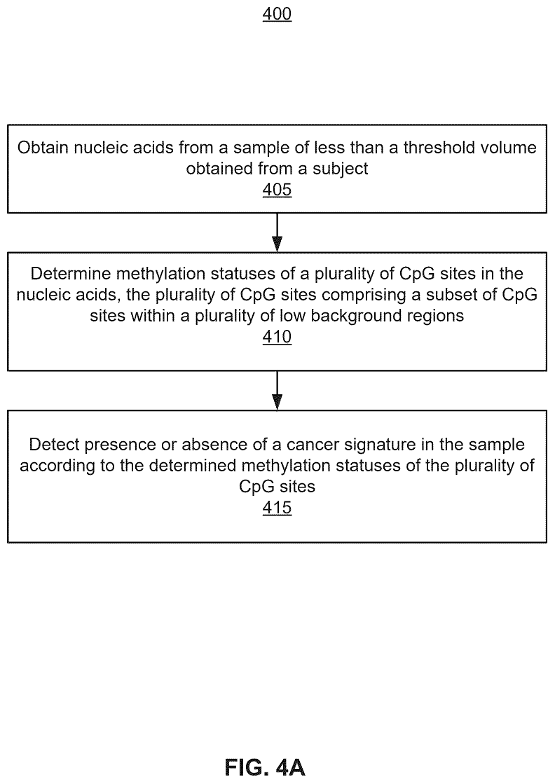

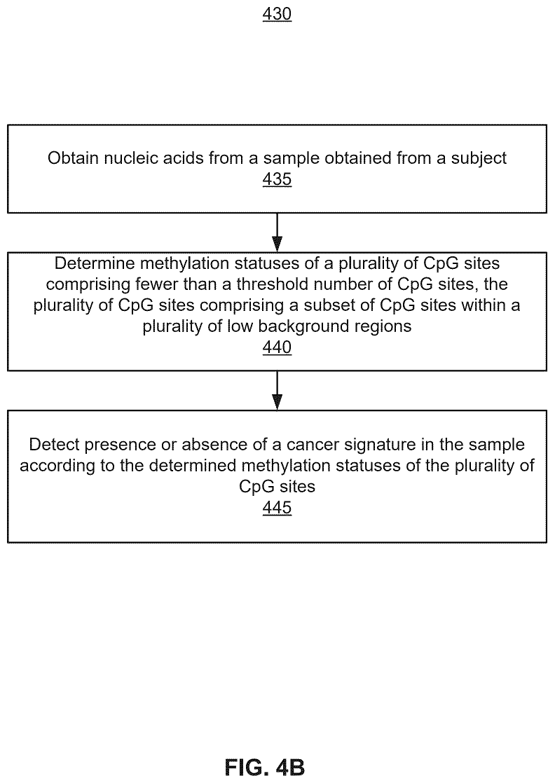

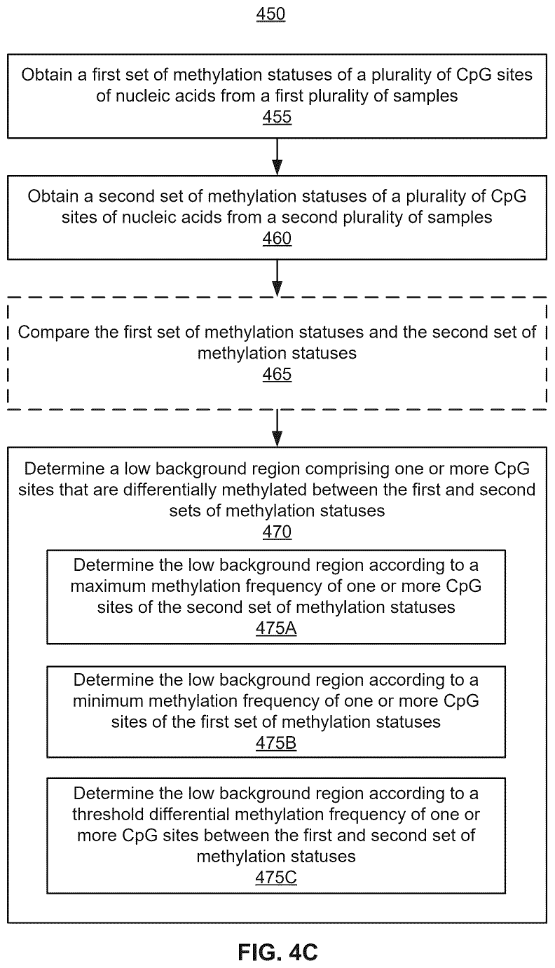

FIG. 1B is a block diagram of the signature detection system for predicting presence or absence of a signature in a sample, in accordance with an embodiment. FIG. 1B introduces elements of the signature detection system 125 which, in various embodiments, includes a low background region (LBR) identification module 150 and a signature detection module 160. Generally, the LBR identification module 150 performs steps for identifying one or more LBRs containing one or more CpG sites that are differentially methylated in cancer and non-cancer samples. Further example steps performed by the LBR identification module 150 are described herein, such as in reference to FIG. 3 and FIG. 4C, for identifying a low background region. The signature detection module 160 analyzes methylation statuses of a plurality of CpG sites comprising at least a subset of CpG sites within a plurality of low background regions (LBRs) to detect presence or absence of a cancer signature in a sample. Further example steps performed by the signature detection module 160 are described herein, such as in reference to FIGS. 4A and 4B, for detecting presence or absence of a cancer signature in a sample according to methylation statuses of the plurality of CpG sites. As shown in FIG. 1B, the signature detection system 125 may further include a data store 170 for storing data, an example of which include training data for training a machine learning model. An additional example of data stored in the data store 170 can be trained machine learning models which can be deployed to detect presence or absence of a cancer signature in a sample.

### Example Assays

As discussed herein, FIG. 1A shows the step of performing one or more assays 120. In various embodiments, performing one or more assays 120 involves one or more steps of 1) converting nucleic acids from a sample, 2) enriching for target sequences of interest of the nucleic acids, and 3) determining methylation statuses of a plurality of CpG sites, wherein the plurality of CpG sites comprise at least a subset of CpG sites within a plurality of low background regions (LBRs). In various embodiments, performing one or more assays involves performing one or two of the aforementioned three steps. In particular embodiments, performing one or more assays 120 involves performing each of the aforementioned three steps. In various embodiments, performing one or more assays involves performing one or more of bisulfite conversion, nucleic acid amplification, polymerase chain reaction (PCR), methylation-specific PCR, bisulfite pyrosequencing, single-strand conformation polymorphism (SSCP) analysis, methylation-sensitive single-strand conformation analysis, high resolution melting analysis, methylation-sensitive single-nucleotide primer extension, restriction analysis, microarray technology, next generation methylation sequencing, nanopore sequencing, endonuclease digestion, affinity enrichment, target enrichment, hybrid capture, or enzymatic conversion.

Performing an assay can involve converting nucleic acids from the obtained sample 115. In various embodiments, converting nucleic acids includes treating the nucleic acids to capture methylation modifications. In various embodiments, converting nucleic acids involves converting one or more unmethylated nucleotides (e.g., cytosines) to another nucleotide (a “converted nucleotide”, as used herein), e.g., using chemical or enzymatic means. In certain embodiments, one or more unmethylated cytosines are converted to a nucleotide that pairs with adenine (e.g., the unmethylated cytosine may be converted to uracil). In certain embodiments, one or more unmethylated adenines are converted to a base that pairs with cytosine (e.g., the unmethylated adenine may be converted to inosine (I)). In certain embodiments, one or more methylated cytosines (e.g., a 5-methylcytosine (5mC)) is converted to a thymine, which pairs with adenine. In certain embodiments, methylated cytosines are protected from conversion (e.g., deamination) during the conversion step.

After a nucleic acid has been treated to convert unmethylated, or, in some cases, methylated nucleotides, into another nucleotide, the nucleic acid may be amplified. During amplification, the converted nucleotide pairs with its complementary nucleotide, and in the next round of amplification, the complementary nucleotide pairs with a replacement nucleotide. For example, following the conversion of an unmethylated cytosine to a uracil, the nucleic acid may be amplified such that an adenine pairs with the uracil in the first round of replication, and in the second round of replication, the adenine pairs with a thymine. Accordingly, the thymine replaces the uracil in the original nucleic acid sequence, and is referred to herein as a “replacement nucleotide”.

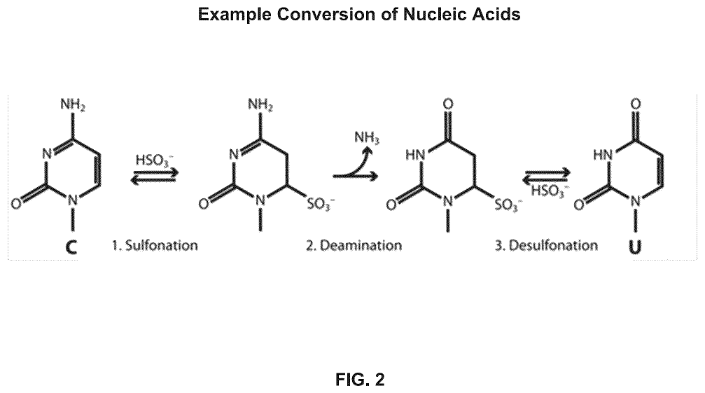

In various embodiments, the step of performing one or more assays 120 involves providing a sample of cell-free deoxyribonucleic acid (cfDNA) molecules from a subject and reacting the plurality of the cfDNA molecules with a deaminating agent to generate converted cfDNA molecules. In certain aspects, conversion of the nucleic acids involves using the deaminating agent to selectively deaminate nucleotides. FIG. 2 depicts an example conversion of nucleic acids, in accordance with an embodiment. Selective deamination refers to a process in which unmethylated cytosine residues are selectively deaminated over methylated cytosine (5-methylcytosine) residues. In certain embodiments, deamination of cytosine forms uracil, effectively inducing a C to T point mutation to allow for detection of methylated cytosines. Methods of deaminating cytosine are known in the art, and include chemical conversion (e.g., bisulfite conversion) and enzymatic conversion. In certain embodiments, the enzymatic conversion comprises subjecting the nucleic acid to TET2, which oxidizes methylated cytosines, thereby protecting them, and subsequent exposure to APOBEC, which converts unprotected (i.e., unmethylated) cytosines to uracils.

In some embodiments, the conversion, for example, bisulfite conversion or enzymatic conversion, uses commercially available kits. Bisulfite conversion can be performed using commercially available technologies, such as EZ DNA Methylation-Gold, EZ DNAMethylation-Direct or an EZ DNAMethylation-Lighting kit (Zymo Research Corp (Irvine, California)) or EpiTect Fast available from Qiagen (Germantown, MD). In another example a kit such as APOBECSeq (NEBiolabs) or OneStep qMethyl-PCR Kit (Zymo Research Corp (Irvine, California)) is used.

Bisulfite conversion is performed on DNA by denaturation using high heat, preferential deamination (at an acidic pH) of unmethylated cytosines, which are then converted to uracil by desulfonation (at an alkaline pH). Methylated cytosines remain unchanged on the single-stranded DNA (ssDNA) product.

In some embodiments the methods include treatment of the sample with bisulfite (e.g., sodium bisulfite, potassium bisulfite, ammonium bisulfite, magnesium bisulfite, sodium metabisulfite, potassium metabisulfite, ammonium metabisulfite, magnesium metabisulfite and the like). Unmethylated cytosine is converted to uracil through a three-step process during sodium bisulfite modification. As shown in FIG. 2, the steps are sulfonation to convert cytosine to cytosine sulphonate, deamination to convert cytosine sulphonate to uracil sulphonate and alkali desulfonation to convert uracil sulphonate to uracil. Conversion on methylated cytosine is much slower and is not observed at significant levels in a 4-16 hour reaction. (See Clark et al., Nucleic Acids Res., 22 (15): 2990-7 (1994).) If the cytosine is methylated it will remain a methylated cytosine. If the cytosine is unmethylated it will be converted to uracil. When the modified strand is copied, for example, through extension of a locus-specific primer, a random or degenerate primer or a primer to an adaptor, a G will be incorporated in the interrogation position (opposite the C being interrogated) if the C was methylated and an A will be incorporated in the interrogation position if the C was unmethylated and converted to U. When the double stranded extension product is amplified those Cs that were converted to Us and resulted in incorporation of A in the extended primer will be replaced by Ts during amplification. Those Cs that were not converted (i.e., the methylated Cs) and resulted in the incorporation of G will be replaced by unmethylated Cs during amplification.

In various embodiments, after conversion of nucleic acids, the converted nucleic acids undergo library construction. In various embodiments, converted nucleic acids can undergo end-repairing and/or addition of library or sequencing adapters. In various embodiments, converted nucleic acids can undergo biotinylation (e.g., addition of biotin moieties to converted nucleic acids). In various embodiments, barcodes can be incorporated into converted nucleic acids, thereby enabling subsequent sample demultiplexing (e.g., demultiplexing to identify sources of converted nucleic acids or demultiplexing to identify a common source from converted nucleic acids). As used herein, a “nucleic acid template” refers to a nucleic acid derived from the converted nucleic acid (e.g., any of a nucleic acid derived from a converted nucleic acid that underwent library construction, end-repairing, addition of library or sequencing adapters, biotinylation, barcode addition, or any combination thereof).

In various embodiments, the converted nucleic acids undergo nucleic acid amplification, an example of which includes polymerase chain reaction (PCR)-based amplification. Here, nucleic acid amplification results in the generation of amplified nucleic acids. Further examples of nucleic acid amplification assays, and in particular, PCR-based amplification, are described herein.

In various embodiments, performing the assay 120 described in FIG. 1A includes enriching for nucleic acid sequences of interest. In various embodiments, nucleic acid sequences of interest include sequences comprising ranges of genomic locations comprising one or more CpG sites. In particular embodiments, nucleic acid sequences of interest include sequences comprising ranges of genomic locations including CpG sites located within low background regions (LBRs), such as LBRs shown in Table 1 (e.g., any of SEQ ID NOs: 1-130). In various embodiments, a nucleic acid sequence of interest comprises at least a portion of a range of genomic locations within a LBR shown in Table 1 (e.g., any of SEQ ID NOs: 1-130). In various embodiments, a portion of a range of genomic locations within a LBR includes at least 2 sequential CpG sites. In various embodiments, a portion of a range of genomic locations within a LBR includes at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 sequential CpG sites. Example steps for enriching nucleic acid sequences of interest can include performing any of hybrid capture, nucleic acid amplification, or CRISPR-based enrichment methods.

In various embodiments, performing the assay 120 includes enriching for nucleic acid sequences of interest that comprise CpG sites within at least a subset of the CpG sites of the LBRs shown in Table 1 (e.g., any of SEQ ID NOs: 1-130). In some embodiments, performing the assay 120 includes enriching for nucleic acid sequences of interest that comprise CpG sites within a portion of the LBRs shown in Table 1 (e.g., any of SEQ ID NOs: 1-130). For example, an example LBR in Table 1 may include a genomic region of ˜450 nucleotides in length. Therefore, performing the assay 120 includes enriching for a nucleic acid sequence of interest that is a portion of the ˜450 nucleotide genomic region. AIn some embodiments, performing the assay 120 includes enriching for nucleic acid sequences of interest that comprise CpG sites within full genomic regions of LBRs shown in Table 1 (e.g., any of SEQ ID NOs: 1-130).

In various embodiments, performing the assay 120 includes enriching for nucleic acid sequences of interest that comprise CpG sites within 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, 102 or more, 103 or more, 104 or more, 105 or more, 106 or more, 107 or more, 108 or more, 109 or more, 110 or more, 111 or more, 112 or more, 113 or more, 114 or more, 115 or more, 116 or more, 117 or more, 118 or more, 119 or more, 120 or more, 121 or more, 122 or more, 123 or more, 124 or more, 125 or more, 126 or more, 127 or more, 128 or more, or 129 or more LBRs shown in Table 1 (e.g., SEQ ID NOs: 1-130). In particular embodiments, performing the assay 120 includes enriching for nucleic acid sequences of interest that comprise CpG sites within 130 of the LBRs shown in Table 1 (e.g., SEQ ID NOs: 1-130).

Referring to the method of hybrid capture, it may involve using a hybrid capture probe set. Here, a hybrid capture probe set can be generated such that probes of the probe set are complementary or substantially complementary to sequences of binding sites of converted nucleic acids. Examples of such hybrid capture probe sets include the KAPA HyperPrep Kit and SeqCAP Epi Enrichment System from Roche Diagnostics (Pleasanton, CA). For example, hybrid capture probe sets can be designed to hybridize with particular sequences of binding sites of converted nucleic acids (e.g., bisulfite converted DNA), thereby capturing and enriching the particular sequences.

Referring to the method of nucleic acid amplification, in various embodiments, a nucleic acid amplification is “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCR) assays, real-time PCR assays, quantitative real-time PCR (qPCR) assays, digital PCR (dPCR), allele-specific PCR assays, reverse-transcription PCR assays, reporter assays, linear polymerase reactions, nucleic acid sequence-based amplification (NASBAs), rolling circle amplifications, nicking endonuclease amplification (NEAR), transcription-mediated amplification (TMA), loop-mediated isothermal amplification (LAMP), helicase-dependent amplification (HAD), or strand displacement amplification (SDA) and the like, disclosed in the following references, each of which are incorporated herein by reference herein in their entirety: Mullis et al., U.S. Pat. Nos. 4,683,195; 4,965,188; 4,683,202; 4,800,159 (PCR); Gelfand et al., U.S. Pat. No. 5,210,015 (real-time PCR with “taqman” probes); Wittwer et al., U.S. Pat. No. 6,174,670; Kacian et al., U.S. Pat. No. 5,399,491 (“NASBA”); Lizardi, U.S. Pat. No. 5,854,033; Aono et al., Japanese patent publ. JP 4-262799 (rolling circle amplification); and the like. In one aspect, the amplification reaction is PCR. An amplification reaction may be a “real-time” amplification if a detection chemistry is available that permits a reaction product to be measured as the amplification reaction progresses, e.g., “real-time PCR”, or “real-time NASBA” as described in Leone et al., Nucleic Acids Research, 26:2150-2155 (1998), and like references. For example, given a converted nucleic acid (e.g., bisulfite converted nucleic acid), primers designed to be complementary or substantially complementary to sequences of binding sites of the converted nucleic acid can be provided. Here, primers (e.g., PCR primers) are added to initiate the amplification of target sequences of binding sites of the converted nucleic acid. In various embodiments, the primers are whole genome primers that enable whole genome amplification. In various embodiments, the primers are gene-specific primers that result in amplification of sequences of specific genes. In various embodiments, the primers are allele-specific primers. For example, allele specific primers can target a range of genomic locations (e.g., a range of genomic locations shown in Table 1) which includes two or more sequential CpG sites. Therefore, performing nucleic acid amplification results in amplification of the range of genomic locations including the two or more sequential CpG sites.

### Exemplary Methods

The methods of the present disclosure involve identifying CpG sites of low background regions (LBRs) and/or using the CpG sites of LBRs to determine presence or absence of cancer signatures in samples obtained from subjects.

### Identifying Low Background Regions

In various embodiments, methods for identifying a low background region (LBR) include obtaining a first set of methylation statuses of a plurality of CpG sites of nucleic acids from a first plurality of samples and obtaining a second set of methylation statuses of a plurality of CpG sites of nucleic acids from a second plurality of samples. In various embodiments, each of the first plurality of samples and the second plurality of samples are obtained from different individuals. In various embodiments, the first plurality of samples are obtained from a set of individuals and the second plurality of samples are obtained from a second set of individuals. In various embodiments, the first set of individuals are non-cancer individuals and therefore, the first plurality of samples are non-cancer samples. In various embodiments, the first set of individuals are healthy individuals and therefore, the first plurality of samples are healthy samples. In various embodiments, the second set of individuals are cancer individuals and therefore, the second plurality of samples are cancer samples. In various embodiments, the second plurality of samples are cancer samples of a common cancer type.

In various embodiments, the first plurality of samples are diseased samples and the second plurality of samples are non-diseased samples. In various embodiments, the first plurality of samples are cancer samples and the second plurality of samples are cancer samples. In various embodiments, the first plurality of samples are cancer samples of a cancer type that differs from a cancer type of the second plurality of samples. Thus, the methylation statuses of CpG sites in the first plurality of samples may differ from the methylation statuses of CpG sites in the second plurality of samples.

In various embodiments, the first plurality of samples comprise at least 5 samples. In various embodiments, the first plurality of samples comprise at least 10 samples. In various embodiments, the first plurality of samples comprise at least 20 samples. In various embodiments, the first plurality of samples comprise at least 30 samples. In various embodiments, the first plurality of samples comprise at least 40 samples. In various embodiments, the first plurality of samples comprise at least 50 samples. In various embodiments, the first plurality of samples comprise at least 100 samples. In various embodiments, the first plurality of samples comprise between 5 and 5000 samples. In various embodiments, the first plurality of samples comprise between 10 and 2000 samples. In various embodiments, the first plurality of samples comprise between 20 and 1000 samples, between 30 and 800 samples, between 40 and 600 samples, or between 50 and 500 samples. In various embodiments, the second plurality of samples comprise between 20 and 1000 samples, between 30 and 800 samples, between 40 and 600 samples, or between 50 and 500 samples. In various embodiments, the first plurality of samples comprise cancer biopsies. In various embodiments, the first plurality of samples comprise cell-free DNA. In various embodiments, the second plurality of samples comprise cancer biopsies. In various embodiments, the second plurality of samples comprise cell-free DNA.

In various embodiments, methods for identifying a LBR include determining the LBR comprising one or more CpG sites that are differentially methylated between the first and second sets of methylation statuses according to a comparison metric. Examples of comparison metrics include, but are not limited to: a maximum methylation frequency, a minimum methylation frequency, or a threshold differential methylation frequency.

As one example, a comparison metric may be a maximum methylation frequency of one or more CpG sites of nucleic acids from non-cancer or healthy samples. In such embodiments, if the methylation frequency of one or more CpG sites of nucleic acids from non-cancer or healthy samples is below the maximum methylation frequency, then a genomic region comprising the one or more CpG sites can be deemed a low background region. In various embodiments, the maximum methylation frequency is less than 10%. In various embodiments, the maximum methylation frequency is less than 9%. In various embodiments, the maximum methylation frequency is less than 8%. In various embodiments, the maximum methylation frequency is less than 7%. In various embodiments, the maximum methylation frequency is less than 6%. In various embodiments, the maximum methylation frequency is less than 5%. In various embodiments, the maximum methylation frequency is less than 4%. In various embodiments, the maximum methylation frequency is less than 3%. In various embodiments, the maximum methylation frequency is less than 2%. In various embodiments, the maximum methylation frequency is less than 1%. In various embodiments, the maximum methylation frequency is less than 0.9%. In various embodiments, the maximum methylation frequency is less than 0.8%. In various embodiments, the maximum methylation frequency is less than 0.7%. In various embodiments, the maximum methylation frequency is less than 0.6%. In various embodiments, the maximum methylation frequency is less than 0.5%. In various embodiments, the maximum methylation frequency is less than 0.4%. In various embodiments, the maximum methylation frequency is less than 0.3%. In various embodiments, the maximum methylation frequency is less than 0.2%. In various embodiments, the maximum methylation frequency is less than 0.1%. In various embodiments, the maximum methylation frequency is between 0.1% and 5%. In various embodiments, the maximum methylation frequency is between 0.5% and 4.5%. In various embodiments, the maximum methylation frequency is between 1% and 3%. In various embodiments, the maximum methylation frequency is between 1.5% and 2.5%. In various embodiments, the maximum methylation frequency is between 1.6% and 2.4%. In various embodiments, the maximum methylation frequency is between 1.7% and 2.3%. In various embodiments, the maximum methylation frequency is between 1.8% and 2.2%. In various embodiments, the maximum methylation frequency is between 1.9% and 2.1%. In particular embodiments, the maximum methylation frequency is about 2%.

As another example, a comparison metric may be a minimum methylation frequency of one or more CpG sites of nucleic acids from cancer samples. In such embodiments, if the methylation frequency of one or more CpG sites of nucleic acids from cancer samples is above the minimum methylation frequency, then a genomic region comprising the one or more CpG sites can be deemed a low background region. In various embodiments, the minimum methylation frequency is greater than 5%. In various embodiments, the minimum methylation frequency is greater than 10%. In various embodiments, the minimum methylation frequency is greater than 15%. In various embodiments, the minimum methylation frequency is greater than 20%. In various embodiments, the minimum methylation frequency is greater than 25%. In various embodiments, the minimum methylation frequency is greater than 30%. In various embodiments, the minimum methylation frequency is greater than 35%. In various embodiments, the minimum methylation frequency is greater than 40%. In various embodiments, the minimum methylation frequency is greater than 45%. In various embodiments, the minimum methylation frequency is greater than 50%. In various embodiments, the minimum methylation frequency is greater than 55%. In various embodiments, the minimum methylation frequency is greater than 60%. In various embodiments, the minimum methylation frequency is greater than 65%. In various embodiments, the minimum methylation frequency is greater than 70%. In various embodiments, the minimum methylation frequency is greater than 75%. In various embodiments, the minimum methylation frequency is greater than 80%. In various embodiments, the minimum methylation frequency is greater than 85%. In various embodiments, the minimum methylation frequency is greater than 90%. In various embodiments, the minimum methylation frequency is greater than 95%. In various embodiments, the minimum methylation frequency is greater than 99%. In various embodiments, the minimum methylation frequency is between 1% and 50%. In various embodiments, the minimum methylation frequency is between 2% and 45%. In various embodiments, the minimum methylation frequency is between 3% and 40%. In various embodiments, the minimum methylation frequency is between 4% and 35%. In various embodiments, the minimum methylation frequency is between 5% and 30%. In various embodiments, the minimum methylation frequency is between 10% and 25%. In various embodiments, the minimum methylation frequency is between 15% and 25%. In various embodiments, the minimum methylation frequency is between 18% and 22%. In various embodiments, the minimum methylation frequency is about 20%. In various embodiments, the minimum methylation frequency is between 6% and 25%. In various embodiments, the minimum methylation frequency is between 7% and 20%. In various embodiments, the minimum methylation frequency is between 8% and 15%. In various embodiments, the minimum methylation frequency is between 9% and 12%. In various embodiments, the minimum methylation frequency is about 10%.

As another example, a comparison metric may be a threshold differential methylation frequency of one or more CpG sites of nucleic acids from cancer samples and one or more CpG sites of nucleic acids from non-cancer or healthy samples. In such embodiments, if the difference between the methylation frequency of one or more CpG sites of nucleic acids from cancer samples and the methylation frequency of one or more CpG sites of nucleic acids from non-cancer or healthy samples is above the threshold differential methylation frequency, then a genomic region comprising the one or more CpG sites can be deemed a low background region. In various embodiments, the threshold differential methylation frequency is greater than 10%. In various embodiments, the threshold differential methylation frequency is greater than 15%. In various embodiments, the threshold differential methylation frequency is greater than 20%. In various embodiments, the threshold differential methylation frequency is greater than 25%. In various embodiments, the threshold differential methylation frequency is greater than 30%. In various embodiments, the threshold differential methylation frequency is greater than 35%. In various embodiments, the threshold differential methylation frequency is greater than 40%. In various embodiments, the threshold differential methylation frequency is greater than 45%. In various embodiments, the threshold differential methylation frequency is greater than 50%. In various embodiments, the threshold differential methylation frequency is greater than 55%. In various embodiments, the threshold differential methylation frequency is greater than 60%. In various embodiments, the threshold differential methylation frequency is greater than 65%. In various embodiments, the threshold differential methylation frequency is greater than 70%. In various embodiments, the threshold differential methylation frequency is greater than 75%. In various embodiments, the threshold differential methylation frequency is greater than 80%. In various embodiments, the threshold differential methylation frequency is greater than 85%. In various embodiments, the threshold differential methylation frequency is greater than 90%. In various embodiments, the threshold differential methylation frequency is greater than 95%. In various embodiments, the threshold differential methylation frequency is greater than 99%. In various embodiments, the threshold differential methylation frequency is between 1% and 50%. In various embodiments, the threshold differential methylation frequency is between 2% and 45%. In various embodiments, the threshold differential methylation frequency is between 3% and 40%. In various embodiments, the threshold differential methylation frequency is between 4% and 35%. In various embodiments, the threshold differential methylation frequency is between 5% and 30%. In various embodiments, the threshold differential methylation frequency is between 10% and 25%. In various embodiments, the threshold differential methylation frequency is between 15% and 25%. In various embodiments, the threshold differential methylation frequency is between 18% and 22%. In various embodiments, the threshold differential methylation frequency is about 20%. In various embodiments, the threshold differential methylation frequency is between 6% and 25%. In various embodiments, the threshold differential methylation frequency is between 7% and 20%. In various embodiments, the threshold differential methylation frequency is between 8% and 15%. In various embodiments, the threshold differential methylation frequency is between 9% and 12%. In various embodiments, the threshold differential methylation frequency is about 10%.

In various embodiments, identifying a LBR comprises identifying the LBR according to two or more comparison metrics. For example, if 1) the methylation frequency of one or more CpG sites of nucleic acids from non-cancer or healthy samples is below the maximum methylation frequency and 2) the methylation frequency of one or more CpG sites of nucleic acids from cancer samples is above the minimum methylation frequency, then a genomic region comprising the one or more CpG sites can be deemed a low background region. To provide a specific example, a LBR can be identified if 1) the methylation frequency of one or more CpG sites of nucleic acids from non-cancer or healthy samples is below a maximum methylation frequency of 2% and 2) the methylation frequency of the one or more CpG sites of nucleic acids from cancer samples is above the minimum methylation frequency of 20%.

As another example, if 1) the methylation frequency of one or more CpG sites of nucleic acids from non-cancer or healthy samples is below the maximum methylation frequency and 2) the difference between the methylation frequency of one or more CpG sites of nucleic acids from cancer samples and the methylation frequency of one or more CpG sites of nucleic acids from non-cancer or healthy samples is above the threshold differential methylation frequency, then a genomic region comprising the one or more CpG sites can be deemed a low background region. As another example, if 1) the methylation frequency of one or more CpG sites of nucleic acids from cancer samples is above the minimum methylation frequency and 2) the difference between the methylation frequency of one or more CpG sites of nucleic acids from cancer samples and the methylation frequency of one or more CpG sites of nucleic acids from non-cancer or healthy samples is above the threshold differential methylation frequency, then a genomic region comprising the one or more CpG sites can be deemed a low background region.

In various embodiments, identifying a LBR comprises identifying the LBR according to three or more comparison metrics. For example, if 1) the methylation frequency of one or more CpG sites of nucleic acids from non-cancer or healthy samples is below the maximum methylation frequency and 2) the methylation frequency of one or more CpG sites of nucleic acids from cancer samples is above the minimum methylation frequency, and 3) the difference between the methylation frequency of one or more CpG sites of nucleic acids from cancer samples and the methylation frequency of one or more CpG sites of nucleic acids from non-cancer or healthy samples is above the threshold differential methylation frequency, then a genomic region comprising the one or more CpG sites can be deemed a low background region.

In various embodiments, one or more LBRs can be ranked to identify which LBRs are of highest importance for distinguishing between samples with a presence of a cancer signature and samples lacking a presence of a cancer signature. In various embodiments, LBRs are ranked according to differential methylation frequencies of CpG sites of the LBRs. In various embodiments, LBRs are ranked according to their significance in distinguishing cancer and non-cancer samples. In various embodiments, LBRs are ranked according to an importance analysis conducted by performing a machine learning model. In various embodiments, the machine learning model is a random forest model.

Reference is made to FIG. 3, which is an example diagram for identifying a low background region, in accordance with an embodiment. FIG. 3 depicts a first plurality of samples (e.g., samples 310A) and a second plurality of samples (e.g., samples 310B). Each of samples 310A and samples 310B may be obtained from different individuals. In various embodiments, samples 310A and samples 310B are obtained from individuals of different categories. For example, samples 310A may be obtained from non-cancer individuals and samples 310B may be obtained from cancer individuals. As another examples, samples 310A may be obtained from healthy individuals and samples 310B may be obtained from cancer individuals. As another examples, samples 310A may be obtained from individuals with a first type of cancer and samples 310B may be obtained from individuals with a second type of cancer, where the first type of cancer and the second type of cancer are different types of cancer.

Nucleic acids of the samples 310A can be converted to generate converted nucleic acids 305A. In the embodiment shown in FIG. 3, converted nucleic acids 305A have a genomic region including multiple CpG sites such as CpG site 315A, CpG site 318A, and CpG site 320A. Prior to conversion, the CpG sites in the nucleic acids from sample 310A may have been unmethylated. Therefore, after conversion, the CpG sites 315A, 318A, and 320A each include uracil nucleobases, as is shown in FIG. 3. Thus, given the presence of uracil nucleobases at the CpG sites, the prior unmethylated state of the CpG sites can be determined. Thus, the unmethylated statuses of the CpG sites 315A, 318A, and 320A are identified.

Nucleic acids of the samples 310B can be converted to generate converted nucleic acids 305B. In the embodiment shown in FIG. 3, converted nucleic acids 305B have a genomic region including multiple CpG sites such as CpG site 315B, CpG site 318B, and CpG site 320B. Prior to conversion, the CpG sites in the nucleic acids from sample 310B may have been methylated. Therefore, after conversion, the CpG sites 315B, 318B, and 320B each include cytosine nucleobases, as is shown in FIG. 3. Thus, given the presence of cytosine nucleobases at the CpG sites, the prior methylated state of the CpG sites can be determined. Thus, the methylated statuses of the CpG sites 315A, 318A, and 320A are identified.

The CpG sites of converted nucleic acid 305A may correspond to the CpG sites of the converted nucleic acid 305B. For example, CpG site 315A on converted nucleic acid 305A may have the same genomic coordinates as CpG site 315B on converted nucleic acid 305B. Similarly, CpG site 318A on converted nucleic acid 305A may have the same genomic coordinates as CpG site 318B on converted nucleic acid 305B. Similarly, CpG site 320A on converted nucleic acid 305A may have the same genomic coordinates as CpG site 320B on converted nucleic acid 305B. Additionally, although FIG. 3 shows a single genomic region of a converted nucleic acid 305A and a single genomic region of a converted nucleic acid 305B, the methods shown in FIG. 3 can involve multiple genomic regions of multiple converted nucleic acids 305A and multiple genomic regions of multiple converted nucleic acids 305B.

As shown in FIG. 3, the methylation statuses of CpG sites in nucleic acids from samples 310A, the methylation statuses of CpG sites in nucleic acids from samples 310B, and a comparison metric 325 are used to identify a low background region 330. In various embodiments, the comparison metric 325 represents a threshold value. In some embodiments, the comparison metric 325 is a maximum methylation frequency. In some embodiments, the comparison metric 325 is a minimum methylation frequency. In some embodiments, the comparison metric 325 is a threshold differential methylation frequency.

As one example, the comparison metric 325 is a maximum methylation frequency of samples 310A (e.g., non-cancer samples). Therefore, if the methylation statuses of CpG sites of nucleic acids from samples 310A are below the maximum methylation frequency, then the genomic region including the CpG sites can be deemed a low background region 330. As one example, the comparison metric 325 is a minimum methylation frequency of samples 310B (e.g., cancer samples). Therefore, if the methylation statuses of CpG sites of nucleic acids from samples 310B are above the minimum methylation frequency, then the genomic region including the CpG sites can be deemed a low background region 330. As one example, the comparison metric 325 is a threshold differential methylation frequency of one or more CpG sites between the first set of methylation statuses and the second set of methylation statuses. Therefore, if the difference between methylation statuses of CpG sites of nucleic acids from samples 310B are above the threshold differential methylation frequency, then the genomic region including the CpG sites can be deemed a low background region 330.

### Using Low Background Regions to Generate Predictions

Generally, predicting presence or absence of a cancer signature in a sample comprises analyzing methylation statuses of one or more CpG sites of low background regions. In various embodiments, predicting presence or absence of a cancer signature in a sample comprises analyzing methylation statuses of a plurality of CpG sites comprising at least a subset of CpG sites within a plurality of LBRs, such as LBRs shown in Table 1 (e.g., any of SEQ ID NOs: 1-130).

In various embodiments, predicting presence or absence of a cancer signature in a sample comprises analyzing methylation statuses of a plurality of CpG sites within a portion of the LBRs shown in Table 1 (e.g., any of SEQ ID NOs: 1-130). For example, an example LBR in Table 1 may include a genomic region of ˜450 nucleotides in length. Therefore, predicting presence or absence of a cancer signature in a sample comprises analyzing methylation statuses of only a subset of CpG sites that are located within the ˜450 nucleotide genomic region. In some embodiments, predicting presence or absence of a cancer signature in a sample comprises analyzing methylation statuses of all CpG sites within a LBR shown in Table 1 (e.g., any of SEQ ID NOs: 1-130). In various embodiments, predicting presence or absence of a cancer signature in a sample comprises analyzing methylation statuses of CpG sites within 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, 102 or more, 103 or more, 104 or more, 105 or more, 106 or more, 107 or more, 108 or more, 109 or more, 110 or more, 111 or more, 112 or more, 113 or more, 114 or more, 115 or more, 116 or more, 117 or more, 118 or more, 119 or more, 120 or more, 121 or more, 122 or more, 123 or more, 124 or more, 125 or more, 126 or more, 127 or more, 128 or more, or 129 or more low background regions (LBRs) shown in Table 1 (e.g., any of SEQ ID NOs: 1-130). In particular embodiments, predicting presence or absence of a cancer signature in a sample comprises analyzing methylation statuses of CpG sites within 130 of the low background regions shown in Table 1 (e.g., any of SEQ ID NOs: 1-130).

In various embodiments, predicting presence or absence of a cancer signature in a sample comprises analyzing methylation statuses of a maximum number of CpG sites within low background regions, such as low background regions shown in Table 1 (e.g., any of SEQ ID NOs: 1-130). In various embodiments, predicting presence or absence of a cancer signature in a sample comprises analyzing methylation statuses of fewer than 5000 CpG sites within low background regions (e.g., LBRs shown in Table 1). In various embodiments, predicting presence or absence of a cancer signature in a sample comprises analyzing methylation statuses of fewer than 4000 CpG sites within low background regions (e.g., LBRs shown in Table 1), fewer than 3500 CpG sites within low background regions (e.g., LBRs shown in Table 1), fewer than 3000 CpG sites within low background regions (e.g., LBRs shown in Table 1), fewer than 2500 CpG sites within low background regions (e.g., LBRs shown in Table 1), fewer than 2000 CpG sites within low background regions (e.g., LBRs shown in Table 1), fewer than 1900 CpG sites within low background regions (e.g., LBRs shown in Table 1), fewer than 1800 CpG sites within low background regions (e.g., LBRs shown in Table 1), fewer than 1700 CpG sites within low background regions (e.g., LBRs shown in Table 1), fewer than 1600 CpG sites within low background regions (e.g., LBRs shown in Table 1), fewer than 1500 CpG sites within low background regions (e.g., LBRs shown in Table 1), fewer than 1400 CpG sites within low background regions (e.g., LBRs shown in Table 1), fewer than 1300 CpG sites within low background regions (e.g., LBRs shown in Table 1), fewer than 1200 CpG sites within low background regions (e.g., LBRs shown in Table 1), fewer than 1100 CpG sites within low background regions (e.g., LBRs shown in Table 1), fewer than 1000 CpG sites within low background regions (e.g., LBRs shown in Table 1), fewer than 900 CpG sites within low background regions (e.g., LBRs shown in Table 1), fewer than 800 CpG sites within low background regions (e.g., LBRs shown in Table 1), fewer than 700 CpG sites within low background regions (e.g., LBRs shown in Table 1), fewer than 600 CpG sites within low background regions (e.g., LBRs shown in Table 1), fewer than 500 CpG sites within low background regions (e.g., LBRs shown in Table 1), fewer than 400 CpG sites within low background regions (e.g., LBRs shown in Table 1), fewer than 300 CpG sites within low background regions (e.g., LBRs shown in Table 1), fewer than 200 CpG sites within low background regions (e.g., LBRs shown in Table 1), or fewer than 100 CpG sites within low background regions (e.g., LBRs shown in Table 1). In particular embodiments, predicting presence or absence of a cancer signature in a sample comprises analyzing methylation statuses of fewer than 1000 CpG sites within low background regions (e.g., LBRs shown in Table 1).PIn particular embodiments, predicting presence or absence of a cancer signature in a sample comprises analyzing methylation statuses of fewer than 500 CpG sites within low background regions (e.g., LBRs shown in Table 1).

In various embodiments, analyzing methylation statuses of one or more CpG sites of low background regions comprises deploying a trained machine learning model. In various embodiments, the trained machine learning model is deployed to analyze methylation statuses of a plurality of CpG sites in nucleic acids, wherein the plurality of CpG sites comprise at least a subset of CpG sites within a plurality of low background regions (LBRs).

In various embodiments, the trained machine learning model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks).

The machine learning model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the machine learning model is trained using supervised learning algorithms, unsupervised learning algorithms, semi-supervised learning algorithms (e.g., partial supervision), weak supervision, transfer, multi-task learning, or any combination thereof.

In various embodiments, the machine learning model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the machine learning model are trained (e.g., adjusted) using the training data to improve the predictive power of the machine learning model.

In various embodiments, machine learning models analyze methylation statuses of a plurality of CpG sites comprising at least a subset of CpG sites within a plurality of LBRs, such as LBRs shown in Table 1 (e.g., any of SEQ ID NOs: 1-130). In some embodiments, machine learning models analyze methylation statuses of a plurality of CpG sites within a portion of the LBRs shown in Table 1 (e.g., any of SEQ ID NOs: 1-130). For example, an example LBR in Table 1 may include a genomic region of ˜450 nucleotides in length. Therefore, a machine learning model may analyze methylation statuses of only a subset of CpG sites that are located within the ˜450 nucleotide genomic region. In some embodiments, machine learning models analyze methylation statuses of all CpG sites within a LBR shown in Table 1. In various embodiments, machine learning models analyze methylation statuses of CpG sites within 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, 102 or more, 103 or more, 104 or more, 105 or more, 106 or more, 107 or more, 108 or more, 109 or more, 110 or more, 111 or more, 112 or more, 113 or more, 114 or more, 115 or more, 116 or more, 117 or more, 118 or more, 119 or more, 120 or more, 121 or more, 122 or more, 123 or more, 124 or more, 125 or more, 126 or more, 127 or more, 128 or more, or 129 or more low background regions (LBRs) shown in Table 1. In particular embodiments, machine learning models analyze methylation statuses of CpG sites within 130 of the low background regions shown in Table 1.

In various embodiments, machine learning models analyze methylation statuses of a maximum number of CpG sites within low background regions, such as low background regions shown in Table 1. In various embodiments, machine learning models analyze methylation statuses of fewer than 5000 CpG sites within low background regions (e.g., LBRs shown in Table 1). In various embodiments, machine learning models analyze methylation statuses of fewer than 4000 CpG sites within low background regions (e.g., LBRs shown in Table 1), fewer than 3500 CpG sites within low background regions (e.g., LBRs shown in Table 1), fewer than 3000 CpG sites within low background regions (e.g., LBRs shown in Table 1), fewer than 2500 CpG sites within low background regions (e.g., LBRs shown in Table 1), fewer than 2000 CpG sites within low background regions (e.g., LBRs shown in Table 1), fewer than 1900 CpG sites within low background regions (e.g., LBRs shown in Table 1), fewer than 1800 CpG sites within low background regions (e.g., LBRs shown in Table 1), fewer than 1700 CpG sites within low background regions (e.g., LBRs shown in Table 1), fewer than 1600 CpG sites within low background regions (e.g., LBRs shown in Table 1), fewer than 1500 CpG sites within low background regions (e.g., LBRs shown in Table 1), fewer than 1400 CpG sites within low background regions (e.g., LBRs shown in Table 1), fewer than 1300 CpG sites within low background regions (e.g., LBRs shown in Table 1), fewer than 1200 CpG sites within low background regions (e.g., LBRs shown in Table 1), fewer than 1100 CpG sites within low background regions (e.g., LBRs shown in Table 1), fewer than 1000 CpG sites within low background regions (e.g., LBRs shown in Table 1), fewer than 900 CpG sites within low background regions (e.g., LBRs shown in Table 1), fewer than 800 CpG sites within low background regions (e.g., LBRs shown in Table 1), fewer than 700 CpG sites within low background regions (e.g., LBRs shown in Table 1), fewer than 600 CpG sites within low background regions (e.g., LBRs shown in Table 1), fewer than 500 CpG sites within low background regions (e.g., LBRs shown in Table 1), fewer than 400 CpG sites within low background regions (e.g., LBRs shown in Table 1), fewer than 300 CpG sites within low background regions (e.g., LBRs shown in Table 1), fewer than 200 CpG sites within low background regions (e.g., LBRs shown in Table 1), or fewer than 100 CpG sites within low background regions (e.g., LBRs shown in Table 1). In particular embodiments, machine learning models analyze methylation statuses of fewer than 500 CpG sites within low background regions (e.g., LBRs shown in Table 1).

In various embodiments, predicting presence or absence of a cancer signature in a sample further comprises performing tissue of origin tracking in a subject. For example, if a cancer signature is predicted to be present in the sample, a tissue of origin prediction can be further generated to identify the tissue from which the cancer signature originates. For example, particular methylation patterns across CpG sites of LBRs can be attributable to certain tissues, examples of which include the nervous tissue (e.g., brain, spinal cord, nerves), muscle tissue (cardiac muscle, smooth muscle, skeletal muscle), epithelial tissue (e.g., GI tract lining, skin), and connective tissue (e.g., fat, bone, tendon, and ligaments). As a particular example, in patients with brain cancer, a first set of CpG sites in one or more LBRs may be frequently methylated. Therefore, if a similar methylation pattern is observed across the first set of CGIs in the one or more LBRs for a subject who is under analysis, prediction can identify that the subject has cancer, and furthermore, that the cancer likely originates from the brain.

In various embodiments, performing a tissue of origin tracking can involve identifying a subtype of the cancer. For example, identifying a subtype of the cancer can involve identifying the cancer as any of an acute lymphoblastic leukemia, acute myeloid leukemia, adrenocortical carcinoma, soft tissue sarcoma, lymphoma, anal cancer, gastrointestinal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, colorectal cancer, uterine cancer, esophageal cancer, head and neck cancer, eye cancer, fallopian tube cancer, gallbladder cancer, gastric cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, small intestine cancer, testicular cancer, throat cancer, thymoma and thymic carcinoma, thyroid cancer, urethral cancer, uterine cancer, vaginal cancer, and vulvar cancer.

### Performance Using Low Background Regions

As disclosed herein, methods involve analyzing methylation statuses of one or more CpG sites of low background regions to predict presence or absence of cancer in a sample. In various embodiments, the disclosed methods achieve one or more performance metrics when predicting presence or absence of cancer in a sample. Example performance metrics include metrics of sensitivity, specificity, positive predictive value (PPV), and/or negative predictive value (NPV). Sensitivity is the true positive rate, reported as a proportion of correctly identified positives. Specificity is the true negative rate reported as a proportion of correctly identified negatives. Positive predictive value refers to the number of true positives divided by the sum of true positives and false positives. Negative predictive value refers to the true negative rate divided by the sum of true negatives and false negatives.

In various embodiments, methods of analyzing methylation statuses of one or more CpG sites of low background regions achieve at least 40% sensitivity in detecting presence or absence of a cancer signature in a sample. In various embodiments, methods of analyzing methylation statuses of one or more CpG sites of low background regions achieve at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sensitivity. In particular embodiments, methods of analyzing methylation statuses of one or more CpG sites of low background regions achieve at least 45% sensitivity. In particular embodiments, methods of analyzing methylation statuses of one or more CpG sites of low background regions achieve at least 50% sensitivity. In particular embodiments, methods of analyzing methylation statuses of one or more CpG sites of low background regions achieve at least 55% sensitivity. In particular embodiments, methods of analyzing methylation statuses of one or more CpG sites of low background regions achieve at least 60% sensitivity. In particular embodiments, methods of analyzing methylation statuses of one or more CpG sites of low background regions achieve at least 65% sensitivity. In particular embodiments, methods of analyzing methylation statuses of one or more CpG sites of low background regions achieve at least 70% sensitivity. In particular embodiments, methods of analyzing methylation statuses of one or more CpG sites of low background regions achieve at least 75% sensitivity. In particular embodiments, methods of analyzing methylation statuses of one or more CpG sites of low background regions achieve at least 80% sensitivity. In particular embodiments, methods of analyzing methylation statuses of one or more CpG sites of low background regions achieve at least 85% sensitivity. In particular embodiments, methods of analyzing methylation statuses of one or more CpG sites of low background regions achieve at least 90% sensitivity. In particular embodiments, methods of analyzing methylation statuses of one or more CpG sites of low background regions achieve at least 95% sensitivity. In particular embodiments, methods of analyzing methylation statuses of one or more CpG sites of low background regions achieve at least 99% sensitivity.

In various embodiments, methods of analyzing methylation statuses of one or more CpG sites of low background regions achieve at least 60% specificity. In various embodiments, methods of analyzing methylation statuses of one or more CpG sites of low background regions achieve at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% specificity. In particular embodiments, methods of analyzing methylation statuses of one or more CpG sites of low background regions achieve at least 80% specificity. In particular embodiments, methods of analyzing methylation statuses of one or more CpG sites of low background regions achieve at least 85% specificity. In particular embodiments, methods of analyzing methylation statuses of one or more CpG sites of low background regions achieve at least 90% specificity. In particular embodiments, methods of analyzing methylation statuses of one or more CpG sites of low background regions achieve at least 95% specificity. In particular embodiments, methods of analyzing methylation statuses of one or more CpG sites of low background regions achieve at least 99% specificity. In particular embodiments, methods of analyzing methylation statuses of one or more CpG sites of low background regions achieve at least 99.5% specificity. In particular embodiments, methods of analyzing methylation statuses of one or more CpG sites of low background regions achieve at least 99.9% specificity.

In various embodiments, the methods of analyzing methylation statuses of one or more CpG sites of low background regions achieve a particular sensitivity and a particular specificity. The combination of the sensitivity and specificity limits both the number of false positives and the number of false negatives. In various embodiments, the methods of analyzing methylation statuses of one or more CpG sites of low background regions achieve between 30% to 100% sensitivity and between 80% to 100% specificity. In various embodiments, the methods of analyzing methylation statuses of one or more CpG sites of low background regions achieve between 40% to 95% sensitivity and between 80% to 100% specificity. In various embodiments, the methods of analyzing methylation statuses of one or more CpG sites of low background regions achieve between 50% to 90% sensitivity and between 80% to 100% specificity. In various embodiments, the methods of analyzing methylation statuses of one or more CpG sites of low background regions achieve between 60% to 85% sensitivity and between 80% to 100% specificity. In various embodiments, the methods of analyzing methylation statuses of one or more CpG sites of low background regions achieve between 70% to 80% sensitivity and between 80% to 100% specificity.

In various embodiments, the methods of analyzing methylation statuses of one or more CpG sites of low background regions achieve between 60% to 90% sensitivity and between 90% to 100% specificity. In various embodiments, the methods of analyzing methylation statuses of one or more CpG sites of low background regions achieve between 70% to 88% sensitivity and between 90% to 100% specificity. In various embodiments, the methods of analyzing methylation statuses of one or more CpG sites of low background regions achieve between 75% to 87% sensitivity and between 90% to 100% specificity. In various embodiments, the methods of analyzing methylation statuses of one or more CpG sites of low background regions achieve between 80% to 86% sensitivity and between 90% to 100% specificity. In various embodiments, the methods of analyzing methylation statuses of one or more CpG sites of low background regions achieve between 84% to 85% sensitivity and between 90% to 100% specificity. In various embodiments, the methods of analyzing methylation statuses of one or more CpG sites of low background regions achieve about 85% sensitivity and between 90% to 100% specificity. In various embodiments, the methods of analyzing methylation statuses of one or more CpG sites of low background regions achieve at least 80% sensitivity and at about 90% specificity. In various embodiments, the methods of analyzing methylation statuses of one or more CpG sites of low background regions achieve at least 90% sensitivity and at about 90% specificity. In various embodiments, the methods of analyzing methylation statuses of one or more CpG sites of low background regions achieve at least 95% sensitivity and at least 90% specificity. In various embodiments, the methods of analyzing methylation statuses of one or more CpG sites of low background regions achieve at least 95% sensitivity and at least 95% specificity.

In various embodiments, the methods of analyzing methylation statuses of one or more CpG sites of low background regions achieve between 80% to 95% sensitivity and between 80% to 99% specificity. In various embodiments, the methods of analyzing methylation statuses of one or more CpG sites of low background regions achieve between 80% to 95% sensitivity and between 80% to 95% specificity. In various embodiments, the methods of analyzing methylation statuses of one or more CpG sites of low background regions achieve between 80% to 95% sensitivity and between 80% to 90% specificity. In various embodiments, the methods of analyzing methylation statuses of one or more CpG sites of low background regions achieve between 80% to 95% sensitivity and between 82% to 88% specificity. In various embodiments, the methods of analyzing methylation statuses of one or more CpG sites of low background regions achieve between 80% to 95% sensitivity and between 84% to 86% specificity. In various embodiments, the methods of analyzing methylation statuses of one or more CpG sites of low background regions achieve between 80% to 95% sensitivity and about 85% specificity.

In various embodiments, the methods of analyzing methylation statuses of one or more CpG sites of low background regions achieve at least 60% positive predictive value. In various embodiments, the methods of analyzing methylation statuses of one or more CpG sites of low background regions achieve at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% positive predictive value. In various embodiments, the methods of analyzing methylation statuses of one or more CpG sites of low background regions achieve at least 80% positive predictive value. In various embodiments, the methods of analyzing methylation statuses of one or more CpG sites of low background regions achieve at least 81% positive predictive value. In various embodiments, the methods of analyzing methylation statuses of one or more CpG sites of low background regions achieve at least 82% positive predictive value. In various embodiments, the methods of analyzing methylation statuses of one or more CpG sites of low background regions achieve at least 83% positive predictive value. In various embodiments, the methods of analyzing methylation statuses of one or more CpG sites of low background regions achieve at least 84% positive predictive value. In various embodiments, the methods of analyzing methylation statuses of one or more CpG sites of low background regions achieve at least 85% positive predictive value.

In various embodiments, the methods of analyzing methylation statuses of one or more CpG sites of low background regions achieve at least 60% negative predictive value. In various embodiments, the methods of analyzing methylation statuses of one or more CpG sites of low background regions achieve at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% negative predictive value. In various embodiments, the methods of analyzing methylation statuses of one or more CpG sites of low background regions achieve at least 98% negative predictive value. In various embodiments, the methods of analyzing methylation statuses of one or more CpG sites of low background regions achieve at least 99% negative predictive value. In various embodiments, the methods of analyzing methylation statuses of one or more CpG sites of low background regions achieve at least 99.4% negative predictive value.

### Methods for Using CpG Sites of Low Background Regions

FIGS. 4A and 4B are example flow charts for using CpG sites of low background regions, in accordance with multiple embodiments.

Referring first to FIG. 4A, it shows a flow chart 400 for using CpG sites of low background regions for detecting presence or absence of a cancer signature in a sample, in accordance with a first embodiment. Step 405 involves obtaining nucleic acids from a sample of less than a threshold volume obtained from a subject. Here, the low background regions have been previously identified such that when they are implemented, only a less than threshold volume of a sample is needed to sufficiently detect a cancer signature in the sample. Thus, the method shown in FIG. 4A need not involve obtaining a large volume of a sample (e.g., a blood sample) from a subject for purposes of detecting presence or absence of a cancer signature in the sample. In particular embodiments, step 405 involves obtaining nucleic acids from a sample of less than 20 mL in volume.

Step 410 involves determining methylation statuses of a plurality of CpG sites in the nucleic acids, the plurality of CpG sites comprising a subset of CpG sites within a plurality of low background regions. In various embodiments, the plurality of low background regions comprise one or more low background regions shown in Table 1 (e.g., any of SEQ ID NOs: 1-130). In particular embodiments, the plurality of low background regions comprise at least 50 low background regions shown in Table 1. In particular embodiments, the plurality of low background regions comprise at least 120 low background regions shown in Table 1.

In various embodiments, the LBRs are identified for satisfying one or more comparison metrics, examples of which include a maximum methylation frequency, a minimum methylation frequency, or a threshold differential methylation frequency. For example, LBRs may be identified for exhibiting a maximum methylation frequency of the one or more CpG sites between 0 and 5% across non-cancer samples, or for exhibiting a minimum methylation frequency of the one or more CpG sites between 75% and 100% across cancer samples.

Step 415 involves detecting presence or absence of a cancer signature in the sample according to the determined methylation statuses of the plurality of CpG sites in the nucleic acids. In various embodiments, step 415 involves detecting presence or absence of a cancer signature in the sample at particular performance metrics. For example, at step 415, the detection of presence or absence of a cancer signature in the sample achieves at least 90% sensitivity at a given specificity of about 85%.

Referring next to FIG. 4B, it shows a flow chart 430 for using CpG sites of low background regions for detecting presence or absence of a cancer signature in a sample, in accordance with a second embodiment. Step 435 involves obtaining nucleic acids from a sample from a subject.

Step 440 involves determining methylation statuses of a plurality of CpG sites comprising fewer than a threshold number of CpG sites, the plurality of CpG sites comprising a subset of CpG sites within a plurality of low background regions. In various embodiments, the plurality of low background regions comprise one or more low background regions shown in Table 1 (e.g., one or more of SEQ ID NOs: 1-130). Here, the low background regions have been previously identified such that when they are implemented, methylation statuses of fewer than a threshold number of CpG sites are sufficient for detecting a cancer signature in the sample. Thus, the method in FIG. 4B represents an improvement as fewer CpG sites are to be analyzed, meaning that fewer reagents and consumables are used to detect presence or absence of the cancer signature. In particular embodiments, methylation statuses of fewer than 500 CpG sites are analyzed to detect presence or absence of a cancer signature in the sample. In particular embodiments, the plurality of low background regions comprise at least 50 low background regions shown in Table 1. In particular embodiments, the plurality of low background regions comprise at least 120 low background regions shown in Table 1. In various embodiments, the LBRs are identified for satisfying one or more comparison metrics, examples of which include a maximum methylation frequency, a minimum methylation frequency, or a threshold differential methylation frequency.

Step 445 involves detecting presence or absence of a cancer signature in the sample according to the determined methylation statuses of the plurality of CpG sites in the nucleic acids. In various embodiments, step 445 involves detecting presence or absence of a cancer signature in the sample at particular performance metrics. For example, at step 445, the detection of presence or absence of a cancer signature in the sample achieves at least 90% sensitivity at a given specificity of about 85%.

### Methods for Identifying Low Background Regions

FIG. 4C is an example flow chart for identifying low background regions, in accordance with an embodiment. Step 455 involves obtaining a first set of methylation statuses of a plurality of CpG sites of nucleic acids from a first plurality of samples. In various embodiments, the first plurality of samples comprise non-cancer samples (e.g., samples obtained from individuals who do not have cancer). In various embodiments, the first plurality of samples comprise healthy samples (e.g., samples obtained from individuals who are healthy).

Step 460 involves obtaining a second set of methylation statuses of a plurality of CpG sites of nucleic acids from a second plurality of samples. In various embodiments, the second plurality of samples comprise cancer samples (e.g., samples obtained from individuals who have cancer).

Step 465 shown in FIG. 4C is an optional step (as denoted by the dotted lines). Step 465 involves comparing the first set of methylation statuses and the second set of methylation statuses. In various embodiments, comparing the first set of methylation statuses and the second set of methylation statuses involves determining differential methylation frequencies of one or more CpG sites between the first set of methylation statuses and the second set of methylation statuses.

Step 470 involves determining a low background region comprising one or more CpG sites that are differentially methylated between the first and second sets of methylation statuses. Steps 475A, 475B, and 475C represent different substeps of step 470. In various embodiments, one of steps 475A, 475B, and 475C is performed to determine a low background region. In various embodiments, two of the steps 475A, 475B, and 475C are performed to determine a low background region. For example, a low background region is determined if the low background region satisfies two of the steps 475A, 475B, and 475C. In various embodiments, each of the steps 475A, 475B, and 475C are performed to determine a low background region. For example, a low background region is determined if the low background region satisfies each of the steps 475A, 475B, and 475C.

Step 475A refers to a first comparison metric e.g., a maximum methylation frequency. Specifically, step 475A involves determining a low background region according to a maximum methylation frequency of one or more CpG sites of the first set of methylation statuses (e.g., from non-cancer samples). Step 475B refers to a second comparison metric e.g., a minimum methylation frequency. Specifically, step 475B involves determining a low background region according to a minimum methylation frequency of one or more CpG sites of the second set of methylation statuses (e.g., from cancer samples). Step 475C refers to a third comparison metric e.g., a threshold differential methylation frequency. Specifically, step 475C involves determining a low background region according to a threshold differential methylation frequency of one or more CpG sites between the first set of methylation statuses and the second set of methylation statuses.

### Example Cancers

Methods disclosed herein involve detecting presence or absence of a cancer signature in a sample using CpG sites of low background regions. In some embodiments, the cancer signature is derived from a cancer, such as an early stage cancer. In various embodiments, the cancer is a preclinical phase cancer. In some embodiments, the cancer is a stage 0 cancer. In various embodiments, the cancer is a stage 1 cancer. In various embodiments, the cancer is a stage 2 cancer. Thus, the methods disclosed herein enable the screening and early detection of a presence of a cancer signature in a sample corresponding to an early stage or preclinical stage cancer. In some embodiments, the cancer is a stage 3 cancer. In some embodiments, the cancer is a stage 4 cancer.

In some embodiment, the cancer is a carcinoma, adenocarcinoma, blastoma, leukemia, seminoma, melanoma, teratoma, lymphoma, neuroblastoma, glioma, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer, lung cancer, and the metastases thereof.

In some embodiments, the cancer is any of an acute lymphoblastic leukemia, acute myeloid leukemia, adrenocortical carcinoma, soft tissue sarcoma, anal cancer, bile duct cancer, bladder cancer, bone cancer, cardiac cancer, central nervous system cancer, chronic lymphocytic leukemia, chronic myelogenous leukemia, chronic myeloproliferative neoplasms, esophageal cancer, head and neck cancer, eye cancer, fallopian tube cancer, gallbladder cancer, gastric cancer, germ cell tumor, gestational trophoblastic cancer, hairy cell leukemia, Hodgkin lymphoma, intraocular melanoma, pancreatic cancer, mesothelioma, metastatic cancer, mouth cancer, multiple endocrine neoplasia syndromes, multiple myeloma neoplasms, myelodysplastic neoplasms, parathyroid cancer, penile cancer, pheochromocytoma, pituitary cancer, plasma cell neoplasm, primary peritoneal cancer, prostate cancer, retinoblastoma, sarcoma, small intestine cancer, testicular cancer, thymoma and thymic carcinoma, urethral cancer, vaginal cancer, and vulvar cancer.

### Computer Implementation

The methods disclosed herein, including the methods of identifying low background regions and methods of using the low background regions for detecting a cancer signature in a sample, are, in some embodiments, performed on one or more computers. In various embodiments, the methods of identifying low background regions and methods of using the low background regions for detecting a cancer signature in a sample can be implemented in hardware or software, or a combination of both. In one embodiment, a machine-readable storage medium is provided, the medium comprising a data storage material encoded with machine readable data which, when using a machine programmed with instructions for using said data, is capable of displaying data and results. Such data can be used for a variety of purposes, such as for determining whether a sample is positive for cancer. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

In some embodiments, the methods of the invention, including the methods of identifying universal cancer signatures and methods of determining tumor content, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### Example Computer

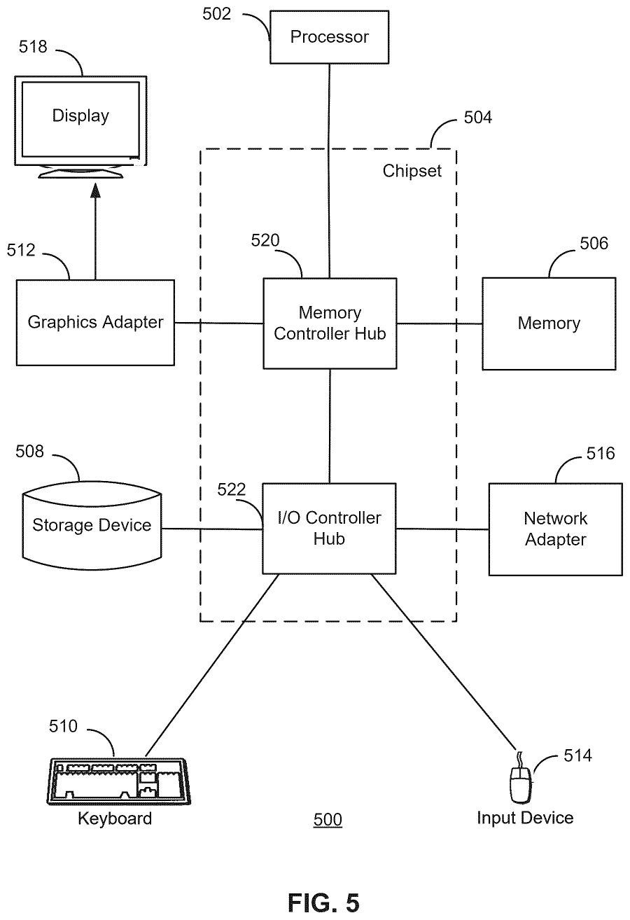

FIG. 5 illustrates an example computer for implementing the entities shown in FIGS. 1A-1B, 2, 3, and 4A-4C.

The computer 500 includes at least one processor 502 coupled to a chipset 504. The chipset 504 includes a memory controller hub 520 and an input/output (I/O) controller hub 522. A memory 506 and a graphics adapter 512 are coupled to the memory controller hub 520, and a display 518 is coupled to the graphics adapter 512. A storage device 508, an input device 514, and network adapter 516 are coupled to the I/O controller hub 522. Other embodiments of the computer 500 have different architectures.

The storage device 508 is a non-transitory computer-readable storage medium such as a hard drive, compact disk read-only memory (CD-ROM), DVD, or a solid-state memory device. The memory 506 holds instructions and data used by the processor 502. The input device 514 is a touch-screen interface, a mouse, track ball, or other type of pointing device, or some combination thereof, and is used to input data into the computer 500. The computer 500 may, in various embodiments, further include a keyboard 510. In some embodiments, the computer 500 may be configured to receive input (e.g., commands) from the input device 514 via gestures from the user. The graphics adapter 512 displays images and other information on the display 518. The network adapter 516 couples the computer 500 to one or more computer networks.

The computer 500 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 508, loaded into the memory 506, and executed by the processor 502. A module can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

The types of computers 500 can vary depending upon the embodiment and the processing power required by the entity. For example, the methods disclosed herein can run in a single computer 500 or multiple computers 500 communicating with each other through a network such as in a server farm. The computers 500 can lack some of the components described above, such as graphics adapters 512, and displays 518.

### Kit Implementation

Also disclosed herein are kits for performing methods disclosed herein such as methods of using the low background regions for detecting a cancer signature in a sample. Such kits can include equipment to draw a sample from a patient. For example, kits can include syringes and/or needles for obtaining a sample from a patient. Kits can include detection reagents for determining methylation statuses of a plurality of CpG sites that comprise a subset of CpG sites within a plurality of low background regions (LBRs). Here, the detection reagents can be used on the sample obtained from the patient.

For example, detection reagents can include a set of primers that, when combined with the sample, allows detection of a plurality of CpG sites in nucleic acids (e.g., cell-free DNA) in the sample. In particular embodiments, the detection reagents enable detection of methylated or unmethylated target sites (e.g., methylated or unmethylated informative CpGs sites), such as methylated or unmethylated CpG sites within low background regions (LBRs).

In particular embodiments, the detection reagents enable detection of methylated or unmethylated CpG sites within at least a subset of the CpG sites of the low background regions shown in Table 1 (e.g., of SEQ ID NOs: 1-130). In particular embodiments, the detection reagents enable detection of methylated or unmethylated CpG sites within 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, 102 or more, 103 or more, 104 or more, 105 or more, 106 or more, 107 or more, 108 or more, 109 or more, 110 or more, 111 or more, 112 or more, 113 or more, 114 or more, 115 or more, 116 or more, 117 or more, 118 or more, 119 or more, 120 or more, 121 or more, 122 or more, 123 or more, 124 or more, 125 or more, 126 or more, 127 or more, 128 or more, or 129 or more low background regions (LBRs) shown in Table 1. In particular embodiments, the detection reagents enable detection of methylated or unmethylated CpG sites within 130 of the low background regions shown in Table 1.

In various embodiments, the detection reagents enable detection of methylated or unmethylated CpG sites including one or more CGIs, such as one or more example CGIs disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. The detection reagents may be primers that target specific known sequences of target sites, thereby enabling nucleic acid amplification of the target sites. Thus, the use of the detection reagents results in generation of methylation information of the patient corresponding to the target sites.

A kit can include instructions for use of one or more sets of detection reagents. For example, a kit can include instructions for performing at least one detection assay such as a nucleic acid amplification assay (e.g., polymerase chain reaction assay including any of real-time PCR assays, quantitative real-time PCR (qPCR) assays, allele-specific PCR assays, and reverse-transcription PCR assays), nucleic acid sequencing (e.g., targeted gene sequencing, targeted amplicon sequencing, whole genome sequencing, or whole genome bisulfite sequencing), hybrid capture, an immunoassay, a protein-binding assay, an antibody-based assay, an antigen-binding protein-based assay, a protein-based array, an enzyme-linked immunosorbent assay (ELISA), reporter assays, flow cytometry, a protein array, a blot, a Western blot, nephelometry, turbidimetry, chromatography, NMR, mass spectrometry, LC-MS, UPLC-MS/MS, enzymatic activity, proximity extension assay, and an immunoassay selected from RIA, immunofluorescence, immunochemiluminescence, immunoelectrochemiluminescence, immunoelectrophoretic, a competitive immunoassay, and immunoprecipitation.

Kits can further include instructions for accessing computer program instructions stored on a computer storage medium. In various embodiments, the computer program instructions, when executed by a processor of a computer system, cause the processor to perform methods disclosed herein, such as methods of identifying low background regions and/or methods of using the low background regions for detecting a cancer signature in a sample.

In various embodiments, the kits include instructions for practicing the methods disclosed herein (e.g., methods of identifying low background regions and methods of using the low background regions for detecting a cancer signature in a sample). These instructions can be present in the kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

### Systems

Further disclosed herein are systems for performing methods disclosed herein, such as methods of identifying low background regions and/or methods of using the low background regions for detecting a cancer signature in a sample. In various embodiments, such a system can include one or more sets of detection reagents for determining methylation status information using a sample obtained from a subject, an apparatus configured to receive a mixture of the one or more sets of detection reagents and the sample obtained from the subject to determine the methylation statuses of a plurality of CpG sites comprising a subset of CpG sites within a plurality of low background regions (LBRs) of the patient, and a computer system communicatively coupled to the apparatus to obtain the methylation statuses and to detect presence or absence of the cancer signature in the sample.

The one or more sets of detection reagents enable the determination of methylation statuses using the sample obtained from the patient. As described herein with reference to the kit implementation, the detection reagents, detection reagents can include a set of primers that, when combined with the sample, allows detection of methylation statuses of a plurality of CpG sites in nucleic acids (e.g., cell-free DNA) in the sample. In particular embodiments, the detection reagents enable detection of methylated or unmethylated target sites (e.g., methylated or unmethylated informative CpGs sites), such as methylated or unmethylated CpG sites within low background regions (LBRs).

The apparatus is configured to determine the methylation information from a mixture of the detection reagents and sample. For example, the apparatus can be configured to perform one or more of a nucleic acid amplification assay (e.g., polymerase chain reaction assay), nucleic acid sequencing (e.g., targeted gene sequencing, whole genome sequencing, or whole genome bisulfite sequencing), and hybrid capture to determine methylation information.

The mixture of the detection reagents and sample may be presented to the apparatus through various conduits, examples of which include wells of a well plate (e.g., 96 well plate), a vial, a tube, and integrated fluidic circuits. As such, the apparatus may have an opening (e.g., a slot, a cavity, an opening, a sliding tray) that can receive the container including the reagent test sample mixture and perform a reading. Examples of an apparatus include one or more of a sequencer, an incubator, plate reader (e.g., a luminescent plate reader, absorbance plate reader, fluorescence plate reader), a spectrometer, or a spectrophotometer.

The computer system, such as example computer 500 described in FIG. 5, communicates with the apparatus to receive the methylation statuses of the plurality of CpG sites comprising a subset of CpG sites within a plurality of low background regions (LBRs). The computer system detects presence or absence of the cancer signature in the sample according to the determined methylation statuses of the plurality of CpG sites.

All publications, patents, patent applications and other documents cited in this application are hereby incorporated by reference herein in their entireties for all purposes to the same extent as if each individual publication, patent, patent application or other document were individually indicated to be incorporated by reference for all purposes.

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

### EXAMPLES

### Example 1: Example Selection of Informative Low Background Regions

To identify informative low background regions, a first step involved defining a starting set of candidate biomarkers. Exemplary candidate biomarkers included CpG sites (e.g., all CpG sites in the genome), a set of CGIs (e.g., 4059 CGIs), or genes (e.g., all known genes or a subset of all known genes).

Next, the method involved identifying a signal for each biomarker across healthy samples, such as healthy normal tissue or non-cancer cell free DNA samples. The number of healthy samples was selected to be representative of the population, such as between 50-200 samples.

Additionally, the method involved identifying a signal for each biomarker across cancer samples, such as a cancer biopsy or cfDNA from patients with cancer. Generally, the signal for an informative biomarker was present across the cancer samples. The cancer samples can be multi-cancer samples or can be specific to a single cancer indication.

Next, the biomarkers underwent rank ordering according to the following factors:
Difference in signal (e.g., difference between signal from healthy and signal from cancer, or difference between signal from a particular cancer and signal from samples other than the particular cancer (e.g., healthy and/or other types of cancer indications)).
Significance (e.g., statistical test+p-value) in distinguishing samples (e.g., distinguishing between healthy and cancer, or difference between particular cancer samples and samples other than the particular cancer (e.g., healthy and/or other types of cancer indications)).
Importance of each biomarker after running a cancer prediction algorithm

The top biomarkers were selected as informative low background regions. For example, a range of the top 10-200 biomarkers were selected as informative low background regions. The performance of identified low background regions achieves a desired level of sensitivity at a given specificity. For example, low background regions achieve at least 90% sensitivity at a given specificity between 85-90%.

### Example 2: Example Low Background Regions

This example describes the methodology for identifying the 130 low background regions shown in Table 1 (e.g., SEQ ID NOs: 1-130). The LBRs were identified within 8.3 mb panel with low mean methylation (<0.02) in normal plasma and high signal in biopsy (mean methylation >0.2). Specifically, LBRs were identified by identifying regions across 60 normal plasma cfDNA samples that had a mean methylation of less than a threshold of 0.02 and regions across 386 biopsy samples that had a mean methylation of greater than 0.2. This was done computationally by calculating mean methylation across overlapping 200 bp regions, and merging together regions that satisfied the threshold criteria. These regions were then ranked by mean methylation signal in 386 biopsy samples, and the top 130 regions (the 130 LBRs) were selected.

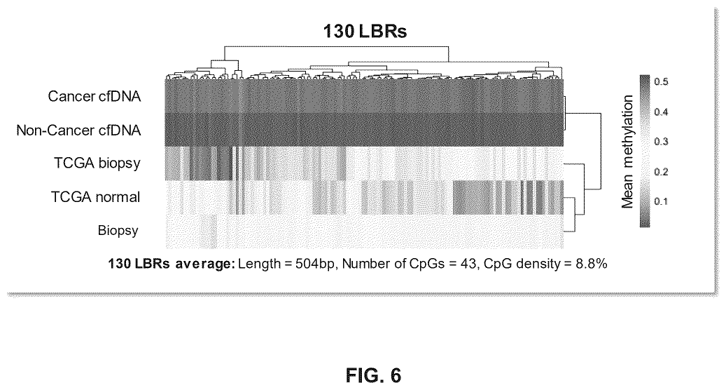

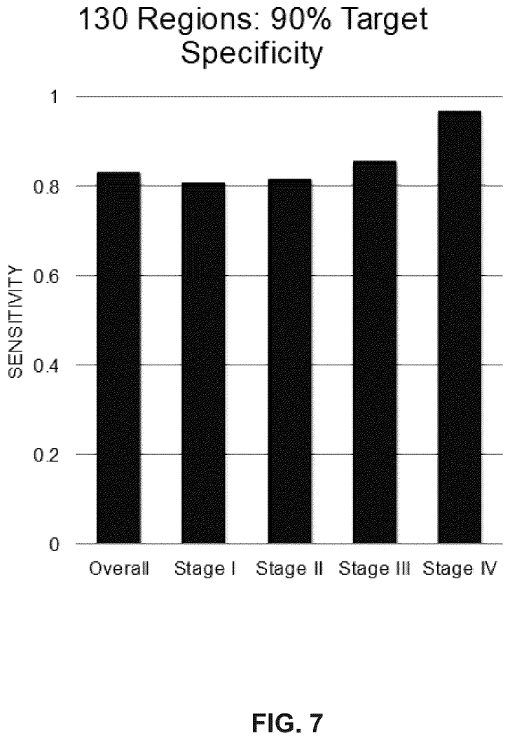

Next, cfDNA data from 1055 samples were in-silico subsetted to generate methylation data for the 130 LBRs. FIG. 6 depicts the differential methylation of the 130 LBRs across cancer cfDNA and non-cancer cfDNA. Additionally, FIG. 6 further shows the methylation of the 130 LBRs across TCGA biopsy and TCGA normal, further validating that the 130 LBRs are differentially methylated in cancer versus non-cancer samples. Using logistic regression, performance of the 130 LBRs was evaluated across different stages of cancer at 90% target specificity. As shown in FIG. 7, the 130 LBRs achieved at least 80% sensitivity across all tumor stages at a 90% target specificity.

The 130 LBRs were further validated by comparing in silico and in vitro results. Specifically, probes for the 130 LBRs were designed and the assay was performed in vitro. In silico read ends were similar to in vitro reads after trimming and mapping. In silico methylation levels and mean in vitro methylation levels were closely aligned across various different cancers (e.g., breast cancer, colon cancer, head and neck cancer, lung cancer, melanoma cancer, and pancreatic cancer) and non-cancer samples. This suggests that the performance of the LBRs described above (e.g., at least 80% sensitivity at a 90% target specificity) would be expected to hold across various cancers when performing the assay in vitro.

### TABLES

TABLE 1
Genomic ranges of low background regions including a plurality
of CpG sites mapped to human genome, hg19. The “Genomic
Coordinate Start” and “Genomic Coordinate End”
columns indicate the beginning and end, respectively of
a range of genomic locations within a chromosome (“Chrom.”).

| SEQ | | Genomic | Genomic |
|---|---|---|---|
| ID | | Coordinate | Coordinate |
| NO: | Chrom | Start | End |
| 1 | chr1 | 119527182 | 119527632 |
| 2 | chr1 | 119530152 | 119530432 |
| 3 | chr1 | 146551368 | 146551898 |
| 4 | chr1 | 1475454 | 1476214 |
| 5 | chr1 | 156863265 | 156863635 |
| 6 | chr1 | 203044592 | 203045022 |
| 7 | chr1 | 221067408 | 221067838 |
| 8 | chr1 | 243646244 | 243646754 |
| 9 | chr1 | 36042742 | 36043444 |
| 10 | chr1 | 41284067 | 41284677 |
| 11 | chr1 | 44883136 | 44884106 |
| 12 | chr1 | 46913816 | 46914343 |
| 13 | chr1 | 48058803 | 48059230 |
| 14 | chr1 | 63795374 | 63795714 |
| 15 | chr1 | 65990821 | 65991721 |
| 16 | chr1 | 67217899 | 67218409 |
| 17 | chr1 | 91183242 | 91183602 |
| 18 | chr1 | 91183712 | 91183962 |
| 19 | chr10 | 101290401 | 101291151 |
| 20 | chr10 | 102894100 | 102894360 |
| 21 | chr10 | 102894700 | 102895270 |
| 22 | chr10 | 103044090 | 103044310 |
| 23 | chr10 | 123922730 | 123923570 |
| 24 | chr10 | 129534690 | 129534920 |
| 25 | chr10 | 16562214 | 16562454 |
| 26 | chr11 | 31825753 | 31826243 |
| 27 | chr11 | 32354800 | 32355390 |
| 28 | chr11 | 32448451 | 32449041 |
| 29 | chr11 | 62693373 | 62693983 |
| 30 | chr12 | 114846931 | 114847561 |
| 31 | chr12 | 52652198 | 52652558 |
| 32 | chr12 | 57618699 | 57619019 |
| 33 | chr12 | 58021294 | 58021744 |
| 34 | chr12 | 6664425 | 6665336 |
| 35 | chr12 | 81102144 | 81102474 |
| 36 | chr13 | 109147608 | 109149208 |
| 37 | chr13 | 112712154 | 112712994 |
| 38 | chr13 | 112720604 | 112721334 |
| 39 | chr13 | 58203376 | 58204016 |
| 40 | chr13 | 58204026 | 58204296 |
| 41 | chr14 | 101193021 | 101193501 |
| 42 | chr14 | 24803748 | 24804358 |
| 43 | chr14 | 37126588 | 37127068 |
| 44 | chr14 | 38724554 | 38725594 |
| 45 | chr15 | 76630599 | 76630959 |
| 46 | chr15 | 76635109 | 76635319 |
| 47 | chr15 | 89922206 | 89922586 |
| 48 | chr16 | 31580559 | 31581023 |
| 49 | chr16 | 54970251 | 54970581 |
| 50 | chr16 | 54970951 | 54971581 |
| 51 | chr16 | 66612869 | 66613229 |
| 52 | chr16 | 67197082 | 67197882 |
| 53 | chr16 | 86612428 | 86612848 |
| 54 | chr17 | 32483927 | 32484257 |
| 55 | chr17 | 37320732 | 37321812 |
| 56 | chr17 | 46618927 | 46619367 |
| 57 | chr17 | 46711072 | 46711352 |
| 58 | chr17 | 5000129 | 5000939 |
| 59 | chr19 | 13210053 | 13210423 |
| 60 | chr19 | 41119211 | 41120121 |
| 61 | chr19 | 46916611 | 46916971 |
| 62 | chr19 | 46996437 | 46997247 |
| 63 | chr19 | 55598337 | 55598657 |
| 64 | chr2 | 105459037 | 105459557 |
| 65 | chr2 | 105459567 | 105459847 |
| 66 | chr2 | 106681872 | 106682322 |
| 67 | chr2 | 114257035 | 114257555 |
| 68 | chr2 | 128421729 | 128421959 |
| 69 | chr2 | 171679398 | 171679898 |
| 70 | chr2 | 171679928 | 171680218 |
| 71 | chr2 | 219735672 | 219736742 |
| 72 | chr2 | 45228144 | 45228934 |
| 73 | chr2 | 468079 | 468609 |
| 74 | chr2 | 63281024 | 63281294 |
| 75 | chr2 | 66808328 | 66809548 |
| 76 | chr2 | 73147405 | 73148085 |
| 77 | chr2 | 80530337 | 80530777 |
| 78 | chr3 | 127795639 | 127796109 |
| 79 | chr3 | 129693197 | 129693897 |
| 80 | chr3 | 129693987 | 129694547 |
| 81 | chr3 | 138657297 | 138657517 |
| 82 | chr3 | 138657887 | 138658147 |
| 83 | chr3 | 138658417 | 138658807 |
| 84 | chr3 | 194118681 | 194118988 |
| 85 | chr4 | 13523882 | 13524592 |
| 86 | chr4 | 140200464 | 140200994 |
| 87 | chr4 | 154713542 | 154714062 |
| 88 | chr4 | 155662869 | 155664039 |
| 89 | chr4 | 174430584 | 174431044 |
| 90 | chr4 | 174459004 | 174459374 |
| 91 | chr4 | 85402970 | 85403220 |
| 92 | chr5 | 115151898 | 115152713 |
| 93 | chr5 | 145725379 | 145725679 |
| 94 | chr5 | 172659649 | 172659909 |
| 95 | chr5 | 37834801 | 37835021 |
| 96 | chr5 | 42994626 | 42994936 |
| 97 | chr5 | 42995122 | 42995415 |
| 98 | chr5 | 45696174 | 45696664 |
| 99 | chr5 | 10381538 | 10381798 |
| 100 | chr6 | 137244265 | 137244585 |
| 101 | chr6 | 1383915 | 1384335 |
| 102 | chr6 | 1393915 | 1394325 |
| 103 | chr6 | 150285942 | 150286515 |
| 104 | chr6 | 1619923 | 1620423 |
| 105 | chr6 | 26240697 | 26240951 |
| 106 | chr6 | 26614073 | 26614293 |
| 107 | chr6 | 26614343 | 26614843 |
| 108 | chr6 | 38682939 | 38683149 |
| 109 | chr6 | 42071932 | 42072572 |
| 110 | chr6 | 6003827 | 6004627 |
| 111 | chr6 | 78172201 | 78172601 |
| 112 | chr7 | 12151220 | 12151550 |
| 113 | chr7 | 156797835 | 156798085 |
| 114 | chr7 | 156798185 | 156798495 |
| 115 | chr7 | 156814492 | 156814593 |
| 116 | chr7 | 24323558 | 24324008 |
| 117 | chr7 | 27203981 | 27205891 |
| 118 | chr7 | 27279145 | 27279415 |
| 119 | chr7 | 42267516 | 42267936 |
| 120 | chr7 | 64349424 | 64349914 |
| 121 | chr8 | 139509105 | 139509455 |
| 122 | chr8 | 23563975 | 23564325 |
| 123 | chr8 | 65282023 | 65282443 |
| 124 | chr8 | 70981763 | 70982053 |
| 125 | chr8 | 70984243 | 70984513 |
| 126 | chr8 | 72468930 | 72469130 |
| 127 | chr8 | 97157293 | 97158030 |
| 128 | chr8 | 97170421 | 97170981 |
| 129 | chr9 | 126774736 | 126775106 |
| 130 | chr9 | 129381027 | 129381477 |

1. A method for detecting a cancer signature in a sample, the method comprising:
obtaining nucleic acids from a single sample of less than 20 mL obtained from a subject;
determining methylation statuses of a plurality of CpG sites in the nucleic acids, wherein the plurality of CpG sites comprise at least a subset of CpG sites within a plurality of low background regions (LBRs) identified for exhibiting a maximum methylation frequency of the one or more CpG sites between 0 and 5% across non-cancer samples and/or a minimum methylation frequency of the one or more CpG sites between 5% and 30% across cancer samples,
detecting presence or absence of the cancer signature in the sample according to the determined methylation statuses of the plurality of CpG sites,
wherein the detection of presence or absence of the cancer signature in the sample achieves at least 40% sensitivity at a given specificity of at least 85%.

2. The method of claim 1, wherein the single sample obtained from the subject is less than 10 mL.

3. (canceled)

4. The method of any one of claims 1-3, wherein the maximum methylation frequency is between 0 and 3%.

5. (canceled)

6. The method of claim 1, wherein the minimum methylation frequency is between 15% and 25%.

7. (canceled)

8. (canceled)

9. The method of claim 1, wherein the plurality of CpG sites comprises fewer than 500 CpG sites.

10. (canceled)

11. (canceled)

12. The method of claim 1, wherein the plurality of CpG sites consist of the subset of CpG sites within the plurality of LBRs.

13. The method of claim 1, wherein the plurality of CpG sites consist of all CpG sites within the plurality of LBRs.

14. The method of claim 1, wherein a quantity of the subset of CpG sites within the plurality of LBRs for which methylation statuses are determined is inversely related to a volume of the sample.

15. The method of claim 1, wherein
determining methylation statuses of the plurality of CpG sites in the nucleic acids comprises performing one or more assays, wherein an assay comprises one or more of:
a. sequencing nucleic acids via targeted sequencing, whole genome sequencing, or whole genome bisulfite sequencing;
b. a nucleic acid amplification assay;
c. a target enrichment assay; and
d. an assay that generates methylation information.

16. The method of claim 15, wherein the nucleic acid amplification assay is a PCR assay.

17. The method of claim 16, wherein the PCR assay comprises a real-time PCR assay, quantitative real-time PCR (qPCR) assay, digital PCR (dPCR) assay, allele-specific PCR assay, or reverse-transcription PCR assay.

18. The method of claim 15, wherein determining methylation statuses of a plurality of CpG sites in the nucleic acids comprises performing the target enrichment assay.

19. The method of claim 18, wherein the target enrichment assay comprises hybrid capture.

20. The method of claim 15, wherein
performing the one or more assays comprise:
obtaining bisulfite converted nucleic acids; and
selectively amplifying target regions of the bisulfite converted nucleic acids.

21. (canceled)

22. The method of claim 15, wherein
performing one or more assays comprises providing one or more probes that bind to the subset of CpG sites within the plurality of low background regions (LBRs).

23. The method of claim 22, wherein at least one probe binds to between 1 and 100 CpG sites within a LBR.

24. The method of claim 22, wherein at least ten probes bind to at least 1 CpG site and less than 100 CpG sites of the plurality of LBRs.

25. The method of claim 22, wherein at least one probe binds to between 10% and 90% of CpG sites within a LBR.

26. (canceled)

27. The method of claim 22, wherein at least one probe binds to every CpG site within a LBR.

28. The method of claim 1, wherein the plurality of LBRs are selected from LBRs identified in Table 1 (e.g., any of SEQ ID NOs: 1-130).

29-71. (canceled)


SEQUENCE LISTING (130 sequences)

SEQ ID NO: 1 - DNA, 450 bp, synthetic construct
  source 1..450: mol_type = other DNA
tcctagctcgaaagttcctctgttgctctgggagagggcgggggagagcaggctcgagagccaggctcctccgaggctggtcttgaggcacttctctagtagcttctccaaaagactgagagtgccggcgtaggtatgacagtgagggtacctcacagacccttctccaaagtctggcgggccttggggtttttcggggccaccaggctcggtggaatttttgaaacgctttcgaaatacatagtttcctctgtggagtgagtgcctacaacgcgcaggccggactgatcccccgttgctgcaggttggtgccccaagctgcgggtgctcgggcgccaactaaagccagctctgtccagacgcggaaagaaaaatgggctgtgaaaaagcaaaaggcctcgtctttgaatgaaagttaaacattaaaatctgaccctagagttgtctaaa

SEQ ID NO: 2 - DNA, 280 bp, synthetic construct
  source 1..280: mol_type = other DNA
cgactggcccttcggcgtcccctctccgcccgcctcccgcggtcggctgcgacgcactcacccgggtgaggctccaggccgtgtgccgccgcgtcctccgtgtctccgagtgggcccgcggggctcagcgcctccatagacaggtccagccccttctcctcccagtctcgcagtttccgttttttatttgagccgatcaaggcttcaacggagaaggcatgtgctcgcgagctcagggcgactgcagatcttctcctttcactcattttagccgcccaca

SEQ ID NO: 3 - DNA, 530 bp, synthetic construct
  source 1..530: mol_type = other DNA
ggctgcgctcacccctgcgatttccccacacgcggggccctagtctgcggtggtgttcgtgctaccccggggctgggttcgcgcacgctcctgacctgccttggctcacggccaacgcggatatcgccgccagagacccttcgccgccctcccagggctcctgagggcgcttccttggtgttctcactgaagctctcgaacagacagatgtgagctctctgtcttttacacgctgaatttggctattggcaaaaaagccctgacccagagcttgggtctccttccgacctgcacacgactccccgactcccgcctccaagcgcggctcttggctcgctggcgggcagcgtccacagagtgtggaaccgccgcagccgcagctcccgcccgctggcgggcagacactagcaggagaaaggacacaaggcctgcgtggtgggaaagcatgggagacctcgctttcccaccggacgagaaggtctccctgcagtctttggacaccagatggatggaggcaccccttctaggaa

SEQ ID NO: 4 - DNA, 760 bp, synthetic construct
  source 1..760: mol_type = other DNA
gagcgtagggagtgtggggatcgcaggttggggaggggagcggaggccggggtgggggagcgcaggccggggaggggagcgcaggccggggaggggagcgcaggccggggaggggagcgcaggccgcacgggcggtaaacaaggcgcgggaggtgggccaggcggcgcgcgcgggcggggagcagggggcgcgcgcgggaagcccctccgctcaccatcacgaccgacgcccccagaatcatgaagatcatggtgttcgcgctcatggacatcgggcggggccgggccgggccggagcgccgccccccggccccggcgcccccccggccccggcccgatgctgagcccccgccgcctccgcagaggtcagcgcttccctggatcgtccgccgccgctcgctgcaaccccggcaccggccggggggaggaggcgcgggggggggggcgccggggagggacgcggggcctttctggggtctctcggcccggcccgccgcgctccgctccgccctcgggtcctccctgcgatgcgctcggctcggctcgactcggctccgctcggctccggccccctccctgcctcctcctccctcctcccccctccctccccgccgccttcctccgcctctcgggagggaccgcgcagaccgcaggccgccccgtgccagcgccgggaggagaccgccatcccccggacgccggccgggacccctccccgggagcctcggcttcgcgtcccgccccagtctggtcagaccca

SEQ ID NO: 5 - DNA, 370 bp, synthetic construct
  source 1..370: mol_type = other DNA
tcctcatccccttcttatccccctctgctcctggcccaggccgcgcagagtcggcctctgtgatcccacccagaatacagttttccctcccgggcgcctggatctcccctcccccggctcctgtttccttgtcaaaacttcctgccttggcgagggcccgagttcccacccccttcctgccccccgcccctcggcgcccctcccggccctgcgatcagcagcgtcccgcctccccgccgctcccgcagtcaggctggggctcgaagtggggaccctcgccccgtcctcggctgtccagtcctcctcctcgcagaccccggcggttcctaccccaggccgcaggggagacggggccccaaggcaggtgagt

SEQ ID NO: 6 - DNA, 430 bp, synthetic construct
  source 1..430: mol_type = other DNA
aggggtgggagtggtgccccttgaaccagcagcgactggccccctccaggcagggcgtgaggatggcaattccaacagggccctcgggcctctggtgtcccccatggtgcagggggatgacaaggtgtttcgccgcgcgccctcctggaggaagcgcttccggccgcgggagcaccacggtcgcggcggcatgctcagcgcttccgcggagaccctcccggcgggcttccgtgtgtccaccctggggaccctgcagcccccaccggccccgccaaagaagatcatgcctgaaggtgagtaacaggcgggctgggcatggccgaggcccagccgagcgcgggcttcttcctggcaccccagggccgggccgggtggagaggggcgaggccgaggctggtgccccgcgctcctgcgctgcagctgcactaac

SEQ ID NO: 7 - DNA, 430 bp, synthetic construct
  source 1..430: mol_type = other DNA
gccttcttccctctttactccattccctaactcctgcctcgtaggcagcggtggggagccgcaggctgctccccgagtcccaacgctactagggtgctgtaacagtgatgaggcggcgttgcagtctggctaggcgctgcagcgggcaccagctacgttttgctgtcggcggcagttacccggacgcaagttaaggggtaggcgcgcagaggcggttggaggcgggcggccggcccctgacctcggagagctgcgccgagcagggcggaaaatttatggtctgtgtacactttctggctccagagccccgcgcaccagaaggatggggccctgcgagcgcgccccaggccgcgagctggggccgcaggcgccacggccatgtatagtgcccacccgactctgttctgcagcgctgtggtcataaaccttc

SEQ ID NO: 8 - DNA, 510 bp, synthetic construct
  source 1..510: mol_type = other DNA
ggggggcttcttgcagcgggggctgctgcctaaacagactccgcgggccccatgatggcaaaccccctcgccccgttcatgacttaatgcctcctagaagaaaaggctaaaaacaagacacacagaacaagaaattgcactctctcaaaacgacagtcgccctgaaacggacgctggaagaggtttccaggcccgtggcgacggtgaggggggtgaaggtcgcaggtggagggcacggggcggcggctgcggagcccggggaggaggcggcgggcgcccgggtcttcctcggagcgcgccccctgggctgggcggcgtgtccctgcctcccgccggcgccctcgtccccgcagcagcgtccccgccgccaccccgctgcctcgtcgcctttctcgcaaacagcggtgccctctacgtgggggatcctggaggctgctgagcacgagcgccctccgcgacccgcccctgctcccgcctccactcttgcctcggggggagggtctcagtctt

SEQ ID NO: 9 - DNA, 702 bp, synthetic construct
  source 1..702: mol_type = other DNA
gcgcagcagcagcaaaggggaaggggcaggagccgggcacagttggatccggaggtcgtgacccaggggaaagcgtgggcggtcgacccagggcagctgcggcggcgaggcaggtgggctccttgctccctggagccgcccctccccacacctgccctcggcgcccccagcagttttcaccttggccctccgcggtcactgcgggattcggcgttgccgccagcccagtggggagtgaattagcgccctccttcgtcctcggcccttccgacggcacgaggaactcctgtcctgccccacagaccttcggcctccgccgagtgcggtactggagcctgccccgccagggccctggaatcagagaaagtcgctctttggccacctgaagcgtcggatccctacagtgcctcccagcctgggcgggagcggcggctgcgtcgctgaaggttggggtccttggtgcgaaagggaggcagctgcagcctcagccccaccccagaagcggccttcgcatcgctgcggtgggcgttctcgggcttcgacttcgccagcgccgcggggcagaggcacctggagctcgcagggcccagacctgggttggaaaagcttcgctgactgcaggcaagcgtccgggaggggcggccaggcgaagccccggcgctttaccacacacttccgggtcccatgccagttgcatccgcg

SEQ ID NO: 10 - DNA, 610 bp, synthetic construct
  source 1..610: mol_type = other DNA
ggaccctccccctccctccccagtcccctgccacatccccagaagtccccgcctccgggtccgtgcgcggggtaggctggctgtgatctcgccgcccccgcccctgcagacttcatcgtgttcgtggcctcggtggccgtcatcgccgcgggtacccagggcaacatcttcgccacgtccgcgctgcgcagcatgcgcttcctgcagatcctgcgcatggtgcgcatggaccgccgcggcggcacctggaagctgctgggctcagtggtctacgcgcatagcaaggtgaggcctgcaagccgcgcgcggagacccgagggcgtgtctggggcacgaccagagcgggcagggcggagagggcgaggtcagaggggctgtctccgtgctgggaaggggtgtggcctgcgggggttggagccctagcaggaggcgaggtctaagcgggtggggcgaagtggattctgaattaggtaggggcgggcctaggagtccggaccggatcagggggcggggccggagaggcggggcttgagggtttcccctggatgcttctgggtttgaagggaccactcgtaccacaaagtgggcagctgaggtgggacttgagggt

SEQ ID NO: 11 - DNA, 970 bp, synthetic construct
  source 1..970: mol_type = other DNA
cggcgcgccgctcgcctggcggctgggctcgctgtagttgtgctcccgctcttcccctgccctcgctccacgccgcgccgctctgtgcggcggctgcctcctcttacggccggaatgcctaatcaccatggtgagaaaacggacaccgcagccgtcctccctcctcgccccgcgccctccctcccatcaattgtcatgcagaagtgatccgggctgccgttttctcgcggccgcccgccagcccctcgccgctgccggcagaagggtggctggtaattgatcttctgggggagggggcgcggagaggcgcgagaactggccccggggaggccaggccacggggtgggccccgttcgcttacttgcaagggacccccagcctccgccaccgaggcggcttgcaagtggtttcagaaatgttggtgatctcgccgtcgctctgcagcacttggaggccgcaggaggactccgcgcgccgcctcgagggccgggagcgcccagcccgcggccgcacacacgagggcccgtcgcgccccccgccctgccccgcctcgccctccacgtccctgcacccccaagtcgcactaagaacccagtccccgatcggtttcctctacgccgtctgagcagaagagagtgggaaccggggtgacggataaggggggggcgcccacgcgacgtcggggtgcatgggagcgcgcgggaggcgctagtgggtgcacggggcgtgagggggacacagcgcgggcgtggggatggccactgcgcggggagggttctgcctggagaaggagggatgggaggaggttgggggagcagggcgcgtggaggagggaggttggacgtgtgtacagcgcctggggacctcgctggccccttggtgcccccaggactctgaggcttctcctttcggcttgaaatgtttttcccttcctgcttttcaaatctgtaaaaatgttccggggaatgtt

SEQ ID NO: 12 - DNA, 527 bp, synthetic construct
  source 1..527: mol_type = other DNA
taggccctgcgccgcccagggaccgcccggcctttgcgtccggcccagctcaagccccttccgccgcgcgcggcctcgggagcgctgggcaggcagcccgggtgggagcgcccacgtctcccgcggacacggacggacggatggacgggcggccgcgcaggcccgcgccccgctcccgccccgcctgccgcgcctcccggggcgcccgcattaaagcgcatatgcaagccatgaattatcaactgaaaggagtcaattaccggctctaaaaacgagtgtctgcgctcgcagcgccccgggccatccgcctatttacggagcgatctaccccgcccggctggggagggggcctcgggagggagagagacggagaaacggagcccgagaccgggagagagccggagaggcagggactggagagtctgggacacgaaggagaggcggggagagaccgagattcagcgagacgcgcggagggccggacgggagccaaggcggctcagttcctcttctcccccgagcaggcg

SEQ ID NO: 13 - DNA, 427 bp, synthetic construct
  source 1..427: mol_type = other DNA
ccaggtgccagggaaggtctcccagcggtcttgcgcggaaggcgttatcagccccgcttattagatgaggcggcgcctgcagtagagggggccgtctccgcagggtctgcagcgccggggcccgcacctcctgctccgcggcccgccgccgccagcccggggctgacctctcagagcagggcggaggattttcacagcacagcttcccgagagagcgggattaagggggaactgctcgggtgggccagagccaggggctagggggaggccgggcggaaacccaaaacctccgcagctgtttccagtttgccgccctggctggaccgcggtggctgcgtgcagagcgagcaaggctgcgcgggcctggagcgcccacggggcgcgggacctctctctgtctccccccgccccgcggggctctctctcg

SEQ ID NO: 14 - DNA, 340 bp, synthetic construct
  source 1..340: mol_type = other DNA
ggtggaactcagctccgggaaggctgtggacctggaccctggggctccccgctgcctgggcgcctctagcccggcgatgagtggttctgcggggaccgacggccccatttccttcttgcctcgtgatgtgggagggaagaaagaaggcggcaactgcaggcccgaagctcccctcgcccagcggcccaaccacagtgctgtcgtgtcggggatcggtccccaagggagcccccaaccccctgcccacgccgagatcacccccgtgccaacctcgagggctcggcctgcgggactccagctgcccctgcctggagggagggaagggagaagagaatacgaa

SEQ ID NO: 15 - DNA, 900 bp, synthetic construct
  source 1..900: mol_type = other DNA
tccctggtatcaggaaactcatgacctcactagttttgatcttttccatttagctttcttttcccactcctcccagttattgcaaccttaattgtagcattttacacaactgctgctgcagagttctggaagtctgggggtgggagggacgggggaaccccagtgcctatctcaagcctgcggtcaccgacgcgggtccggggtgggggggtactgccagggaactcgcggcaccgcgtgcttgctgagttcgcctgggctctcggcctccctgcgggcaggggtcctccctccccagcgccgggtctctcctcccgcaggcccgcggccacccgcagcggcattccccgccgccgcccccgccacctgcgcgaccgccctagccgctcgagtccgctcctcccgctcaggggcggctgcggagggcgcggggcgcacgcgggcggccacccgactcttccctcccttctccccaccccagcctgctcctgctcagccccacctccccacgcactcggctgccctcctcctctcctgagagttgccccgcaccttgggcgaggagttcggagcggccccatcgcagagcccacggccagccgagcgcgcgcgacgcaggtgcccgagccccggcgccgccgccatctctgccttcggtcgagttggacccccggatcaaggtgggacctgcttccctctccgacaccccctattgccacctcttcccgctctgggtaactcgagaatggggctttgggacgcgcgtggcagacgcggagccccgcgggccgcttagggactgggagtccggggcagcttagcgggaccaccagaggggccagcgcccggcgggcggcgggggtggggtgggtgcgacgccgggcgactctcggctcccgt

SEQ ID NO: 16 - DNA, 510 bp, synthetic construct
  source 1..510: mol_type = other DNA
tgatgaggggccctaggggtgcaaatgtctttagtcccaaatgtcttcctcgctacacaatgaaccagttcccctccagctcgccgccaacccaggacgcagcccggctcagagtgacccccagtgggcagtgaagatgagatcctctacatccttagggcagctcagagggccactcagcggccaagtccggccggggagggccgagggctctgtttgaggtcaccctggagaccggcctcagagtccagggagagtgcgcgggcggccgccggctgaatgaagcctgggacgcgggagccgcgccgcgcgcagtgtctgcagtgccggaggtctgggaggctccgggcgaagcctccctgctgcaggtagggtcgcctctctctgcagcgcgtctggaccccaaaggaaggatagggggcaagggatcggccctttgttgagaaatggccctacttggccccgatctctccgccaccttattcctcctctccccaacacataagaaaa

SEQ ID NO: 17 - DNA, 360 bp, synthetic construct
  source 1..360: mol_type = other DNA
cgaagcccgggcggccgcgcgccctcggaactcccccgcggctcccgaggtggcagccgcgcgccactcggcagccccgtagccgagtttctgaactttcttggaaagttgcggggcagacacggccgcagcggttgcggcaacccgaggaggaggaggaagaggaggacgacgagaaggagcaggcacagaaggaggaagcggcggagacgcgatgccgggcgactccggccgctgccgggcgcgttcgcttgtaatccggctgctggcgggcggcgccgaccccctcccgtgacgtcacggccactaccgccgctccccgcgccgcgccgcgccgggcccgcgcccccgccaccgagg

SEQ ID NO: 18 - DNA, 250 bp, synthetic construct
  source 1..250: mol_type = other DNA
tcggactccgctgctcttttgagaacccgcacatccctgagcccgcagccctgagccgaactccgagtccagtggtcccggtgtccacaatcacgagtgcgaggcaaagggagagggaggaagagatcagagggagaccagagaaagggaaccaaaggggagaaagaaaaaaaaaagtagcaaggattcgcccgttttcttaagtcagaagagagagagaagaggacgggaagggacttacctttaggct

SEQ ID NO: 19 - DNA, 750 bp, synthetic construct
  source 1..750: mol_type = other DNA
aagcaagggaatgtaacaaggagaaatgcaggcttcggttggaaactggggagagagagattgtttcgatagcctttctccttttctttttccagaatcatgaaatcaccgaggagaagaaaactggggactagaagagagcagaaacatttcccctgagacagttttgccccggaggtggggtggggaagtggcgctggagctcagcttcctgcttttatgctcggcggccaccgtgatgcacacacactcgaggtccgccaccccgcagtgccgtgcacccctctgcccccacctcggaatttttgtgcccgagcgctcgggatccacggcaaaaccaggcacattcctccctctgcagactcaccgcctggtttgtagacactggtctccgcctggggcgtcaatagtttcaggatttaaagcagaaagacaccgggcgcccctgtggctgaggaatggcggttccagcacaagaccctgcccacagagggtctctccctactgcctgcgggccgccgctaccctaagtactccggggcgcacgtggcccgcgcggcatcccagctccccgggcggacccaggggctgcctctccacaacagttccgctcccttctgcagagccccgcctctcccacctcccgacaggctgttctttctcccttcaaccttgggcacccactcctgtcgccgttgcgggacccgcgtggcagccctccaccctcgtacccactcccagccggtgtct

SEQ ID NO: 20 - DNA, 260 bp, synthetic construct
  source 1..260: mol_type = other DNA
aacctggttccagaaccggcggacaaagtggaggtgagcaagcggggcgggccggccgcccgcgagcggcgcggtctcaggcagctctcggttcattggcctctcgtggggcgcacatactttttccgctcgcggtttctgatcctttcggaggagcgagctcccgctaggcttgcggggagctggaagcaaccgaggccgatagctgggatggggctgaagagccctggctctgttttaccggaggcttcagggctttc

SEQ ID NO: 21 - DNA, 570 bp, synthetic construct
  source 1..570: mol_type = other DNA
gggtttcgcgggtcccactggaaggggaatcaattaggagcagatgggtgtgtgtgagaaagagaattttccttctcccatagccacaactctagttactacgcaatgggctttttgttttgttttgttttttcacatttacgctcacggacgtgaagcgtccccaacactctcgagcagcctccctctccgcgcgcactgacgctttctccggctgcaccctctggcacgctgcaccctctggcacgctgcaccctcccgcacatctggcccttgcccgccggagtttctcccctagctcaggccccatggggtagcgcgggagagtctgggcgaagtcggcggcgccgagacgggcgggccttggggcaggaggaaggaattggagtttcctctttttctgaacgaaggcgaggaatctgcctgggattccgcctacggggccacaaaggaagccatggccccacgattctaccacaacatttctgacgctgggaaaggagtgggggatgcggcctcagaccttggaacgctaaagagggagccagggctagctcgggggttgggggg

SEQ ID NO: 22 - DNA, 220 bp, synthetic construct
  source 1..220: mol_type = other DNA
gctccgcgcgcagctggagcccggcgagagggccgcggaaggggggtgcgaaccggggccggaccccggggaggagccgggaggcgagcggcgaggggcactgcgcggctgggtctgccccggggtttcgcactgcgccgcgggtcgaagtaccgcgagttggccctgactgtctgcaggatgagggtgtcgaggagggttccaggccagcgtgcctgcc

SEQ ID NO: 23 - DNA, 840 bp, synthetic construct
  source 1..840: mol_type = other DNA
ccgctgcccactgcagcctatttacctgttcctccagccatccccttagtcctgggccgggagagaccatttcctccttcctggagggatgagccaacagggctgagccccctactctctcggtggtcagcccgcccgaccaccacgcccctcctttctagggccccagctcccgggccgcggctcgtttgcatttcaaagctacacaatgcgggcggtcccggcgaggcgggaggggccggggcagagggagggtcttcgcctccccacccccagccccgtttccggagcccgcgatggagctttgtgaagatgcaggcgttggggctgctcggcgatgatgacatcatcgtgtcacacacaggagccggctgccggggggtttaagagaagagcctttcggaccacaccggcgctcacgctcatacccgcacgccccgggcagagccgcgcacgccggccacactcgggcgcgcgccggccacactcgcgcgcacacatacgcggcgctcgccccccggcccccggctcgggccgcgagtcgcagctccctgccgccgctcccgccgccacggatgcccgcagctgctcccctctgcagtgcagcaaccccggccgccggccggctcgccccggctcccggctgcaggaatcgcgccaggacgctggccccgctcgcggctagcttgcacgccagggcacagcgaggatgggagggtcgcagtccctgcagccagccccagccagcgacctgaacctggaggcttccgaggcaatgtaagtgctccgcccgggccgccccgagtctcccggggaggaggagaggatgcccgggctttg

SEQ ID NO: 24 - DNA, 230 bp, synthetic construct
  source 1..230: mol_type = other DNA
gcgcccgaggtcacagcgcggagaaacctggcggggccccggactccccggcttgggaaaagcgatgactgccctgaactgctggggcgttcgaaatttccagggtcccgaccctccgtggggtacgcgcgacttcggcgcagatgtcagtccgctgccttccgggttgagggagcgaggactccagacgaccccagggccgctgtccaggcccagccccgcgttctcca

SEQ ID NO: 25 - DNA, 240 bp, synthetic construct
  source 1..240: mol_type = other DNA
agggtcgcgctcgcaggggcgcgcaccgatcgagggtcccagactccggaactgccctctccaggccgcggggcgccctcctgcgcgcacgacccccgcctctccagggtgggacggcgtccccccttgccgtcgctccagggagcccggccgccgcgcccttcctccgcgggctcccacccccatttccggggtctcctccctctcgcccgcaccttcccgcgctccctccccgccctc

SEQ ID NO: 26 - DNA, 490 bp, synthetic construct
  source 1..490: mol_type = other DNA
tccggatcatgcatggaaggctaattgaaaagatcagtcggggcgcttgtggcagccttgtcactttgtgacagtgctccgctccgggaaattgcatcgtcacgacaaacgggaccgtgataaaacgaccctttccgtccttatttgtagatcactcagacgagattgaactgcacttgtttccccttcgaggggagccgcgttttcagggtagccgaaggcttggggctgagggggggccctcaccaaggcgcgggtgggggccggagcctcaactcgatgagaagtgacaggcgtttgggggatctgggctccggccgggaccagcgcaagcagggactttgcggggacaccgcttctccaacagagcaaggcctggcccacgtttccggtttctcctaacttccttttattgccttcctttgcttcgcaagttccatctacccctccagctacagagccccacctctaggcacaggaagcttcccgg

SEQ ID NO: 27 - DNA, 590 bp, synthetic construct
  source 1..590: mol_type = other DNA
ccctttttctctctctggacagaatttcttggggatccttcgccccccacccccacccccggccgtatttctccctaggctcttattaaaaccaggtgcacggaaggcggcccattctggacagaaaagctttcgtttgacccaaagctcccccttccggcgtgttttctagagggaattcgtgagctgagccgaggagcgagcaggcggcttctcctgaccggggagacgcgggaggtttgcaaacagaccttggcctgggctgcgcagggacaaagtgttcaagggcagcaggggaggctgagggagtacacggctgggaacaagggacactgccgcccccagccacgcaggagcggtgtctgcaccactgaaagtcccccggggcccacagctctatccaggcggaagggaagcagccaggccgccgcggatctgaacttgaatcccgggtggaccggacgccccggccggcgctcctgacgcgcggaggagctggcgagaacctgcagatctcagggctcgcccgcggtccggcgctcggttcaggacgtccgggaaccccgcagcgcttaccctctgcagcaggc

SEQ ID NO: 28 - DNA, 590 bp, synthetic construct
  source 1..590: mol_type = other DNA
agtcccccaggcgcggccactcccccacccaatctcccagggcaggcgcttttccttccttcatgacctcactacctccttcccttgacgtcagccccgggtccccgcgcggaggggagggaggatggggatgggtttgggggcagccggtgtcaggatccccgagcaccagggaggagggggctatgaggtcatgcgcgcccaatcccgggccgcacaggcctcgcgctgctgctccccggcccccgccccaacttccagccccgtgcgggctggtgtgcgccgcagttactaggcaaccctatcgcagacgcgggcggccggccaagttcacccaaagttgaagaaaaaatttgggggagagaaggtggagcgagaggcgagaagaggaagcacggagcggggaaaagcttggaagggaatgaagctggccttgggcctcccgctcacaggaagaaggtcccacgcgggccagagcagcctccccgcagggccctgttgcgacccgatctccgtcctgcaccccggccgggggcccaccccagagcgtagcaggggcgctgggaacgacgtccacaaagcgctgccac

SEQ ID NO: 29 - DNA, 610 bp, synthetic construct
  source 1..610: mol_type = other DNA
cgccgtccgaggcgagagcggacgcggccacaataaaggcatcttattgggacaggaaatcgggattttccgagtccggtttcgaaattgaggccttgtagacgcttcacctttcctgcccggctacgtgctgggctccggagggcagtgggagtgcggagaagcagctccactccccagccccaagtcttgcgggcagttcccgaagaaaagatgggtttggggcggtcgcgaaagcggcgcctcgcgtgttttcctgccgttcccgggtccttatagcccggccggagactccgctgagttgactcggcgcccggggtcctgccaggggtagtgaatgctgggaactgagataacgcaacgtggcgtttcggactccccgctggaccgggccgtggagaggagctgcgttcccacagggcatccctaagcgtcctctgtccccgcctatgaggacacaacctggaagtccaggccactggttgccgccttggagttggatgtaccgtgaaagtgcggccccgacgcaggaagcaggcaccttcccccgcactatggaagctcgcgcccctcgcttctcgaaacacctgattcacggcgtcgaccctgc

SEQ ID NO: 30 - DNA, 630 bp, synthetic construct
  source 1..630: mol_type = other DNA
ggcctggctccaggcccggtcctgtaaggactggaacggcagcaaaatgtgcagggaggcagtcggccggcagagctgcggcgggagccaaggtcaggcccgcggggagagcgggcagcttccagcgccggccacaagctcccaggccagctgggccgcagacccctttgcttccagagagcacaacccgcgtcctttctctcagccaggctgcagtggctgccccgagcttcgctttcgtttcccaagctgttaataacgatatgtccccaaatccgaggctcgtgtttgctcccagatgccaagaacgcaacccgaaatccttctcccaaaccctaggtcgacgagatgagttcctacttgacctctgagccgaggtgggccggaaaccgaggcctaggccccgccggggctgcaaggaaaaggggaaactccgagcgtagcgtcttttccttgtggttcctttctccggcatcccggactgcgggccctgcagccacctggaccggcattcaaaggattctgcaagtccagcttcacagactggctttcccagacgctccgaagcccgcaccacgaacagaataaaggagagacgagagatcgcaactagatttgagaatcctcgtt

SEQ ID NO: 31 - DNA, 360 bp, synthetic construct
  source 1..360: mol_type = other DNA
tgaggccgcggtagcaggagacgccgcggtagggggcggcggtgatgcagcagcggccgggccggggcccgcaggccgagatgcagctgaaggcgcggaccccgcagggggcgctgaggcgggagtagaggcaggacatggtgcggtggcggcaggggcgggcggctggaccccaagccggcgctgcgccttatatgacggcccgcgggcccggggagggagctcgcggccaaggcgtttacgagcttggagggctcggccctcgcgcctcgcggagccgggaatgaaagcgagccaggccgcccggccgcctgcataaaacggcgttcgcctcccgcccgagttttatggggccgggaa

SEQ ID NO: 32 - DNA, 320 bp, synthetic construct
  source 1..320: mol_type = other DNA
tacctggggctgcgccccgccgcggccggagcgggtgcccccggccagcagctcccagagccaaggtcttcggacggcctaggcgtgggccgcgcctggagctgggcctggccgaccaaccacacgggggcgctggcccgggcaggggcagccggggcgttgcccgcgcagcgcaccaagaggaagccgtccatcaaggcggcgcgcgccaaaaagatcttcggctggggggacttctactttcgggtgcataccctcaagttttcgctgctggtgaccggcaagatcgtggaccatgtgaacggtaccttcagtgtgta

SEQ ID NO: 33 - DNA, 450 bp, synthetic construct
  source 1..450: mol_type = other DNA
cgctggcggctgggcgcgggtgcagcctaggaggagaggcgcgtgaccgtgcgccagctccccgtggggctcctgccagggtcgaccgggagggggtgccactcacccagatgagccacgcggctgaggcgggggtcgaaaccgacctcgcgcaccttgtcagtccgcgccaggaagaagttaaccacgccgtcggtgaccacgcagcctgggaagccgacgagctcgtggtggaagccgcgcctttgccggaggcagttcccgaggcctggggcgccgggctccacgctcagcagctgccgataagtggtggcaaagccggagatctcgcgcaccgcgccccccacctgcagggagagggaggttgcctccaggcgggcctgggataggggacccgaaggggtcaaggtctgcgctccggtgccttcgggggtacccctgccccatcct

SEQ ID NO: 34 - DNA, 911 bp, synthetic construct
  source 1..911: mol_type = other DNA
cgcttgtactcgtcccgctcccgcttcactttggccagcacgttgtagagagcgcggatctcgggcgtgatggtgtcgatctggacgcccaccccatccgggtgcacccacgacaagccaggcccttgcaccagagtgggctccagtcccggcccgagccggcgggcgtgcgagaacgaccagatggtgccgggcatgaagcgggccgacgaggaataggtggtggaggtggaggtggaggacgacgagaggctggccgcggagggcgcgagggggccggccggggagcgcgcgggcgagcccagcacgcgcgccgaagggctgcagacagcggccggccgggcgcccagctgcagccccaggggccggatggggctgacgaagccggtctgcactgcctggtcgcgacgacccaggccccgccggccctgcttaccctcctccagcgcttgctgcagttgcttctccaacagccggttccggcgctccagctcatgcaccttggccaggaagcagcggaaccggaggttcagggtcttgagcacgttgatgttggagcccaggtcattgcggagggccatggcggcaggcggggccgggcccggcccgggcggcgagtaggcagcagggccggccggcgagagaggcgccgggggcaagtctcctcccccggcgaagtggtcgcctcccagtgagtcccccagtggcccggccaggccctgctgctcctgctgcaggaggaagaggttggggccgaataacggattcatggctgcgccttctgctgggagatgcagaccggtgcaggagcagggatggaaggcgagccagaagagccaatgcggcgccggcgggacagagccgaccaatcaggcggctcggcagcggggcagaggtcagggggcgggccg

SEQ ID NO: 35 - DNA, 330 bp, synthetic construct
  source 1..330: mol_type = other DNA
tctaatgaacccccagtgacccaagaagaccgggtgcttgcaataggcaggaaatgcgtacccggcccgaggaagcaggaaagccgccccccaccccaccccaaccccggaaccgatgtttctttcctaatctgccgctgccttggttttccctccagcttgagggtgcggatttcctgcgcacctgcagctcccagtggccaagtgtttccgatcattccagggggctcgtgataacggctaaggaaggtaaagtaaaagggctctgggccgcaccagagaaaatccgggaggtggataggatgcttgggccgagagggctcgaagcga

SEQ ID NO: 36 - DNA, 1600 bp, synthetic construct
  source 1..1600: mol_type = other DNA
ctgttaggactccccagggggacctcggctccaggtcacagctctgggcactcccttctcctcctgctcccaggatcttgctaaagtggagacggaaggaaaagtttcaggagtgagctgcgttcttagaattgtagcagagagagaggagggagagagcagaggatcggagggcaggagtggagagaggcgcggtctcctggaacgcgcgggggaacgtggaaaggagggcgcgggagacccggagggctgccccggcggggattagaagcgaggcgggttcagcgtcctgggcgccgcaggaaagtgacccgcagagcgaggcgcggcgccccgggagctggagctcagccccacgacccgcgctgcggcgggcaccacgcggggggcgcgcggggaggggagaggcggggccggcggggactgtgtcgccgccgacgccgcggctgcgggtcgcagaggcgggcagagagagccgccgccgagcgggtggcggagcagtccccagcctccagccggcctggctgcgcgcaaccgcgccggccccgggcacaggggcaactgccgacccctctcacccgccgcgcggggctggtttccgagccgagtcccggccatcgccgcgtcgcctggcgcccggctccggcggacccttctccggcaggtaggggcgtcggcggggcgctgcgcgcggtccaagcgcggggaacgcggcggggcagcctctccgagtctggaggtacgcggggcgcagaggctgttctgcaccgccgggctggggacgccgggagggtgccccgggtcggacttgcggcgctgggtccccacccagagttcccgcacggtgagggttggacgcgtctggggggcgtggagaggaggcttgtcggtcccttcctgggaaaggaatcgcggggacttcccaaccgctaggacgcgtaagccgaggctgggagtagaagccttctgaactctggacctaccccagaaggagggggcgaaactggccatttcgagggacgcggaagccccgttgctaggggagatgggggaagcggcgtggtcacctacacaaccgctcccaggcgcgatgggggctctgcttcctcctcctctgtttctttggggatggaaaagagagcagaacaaagatgtagagtgaaggagggctccccagctgttgcctcttgcgcggattctgctgacccctctgcgcgaccccagctgggcagtgtcagctccattgtctcagaggccggcggccaagtgagggaccagtctcacccggggggcgtcgagcctcagcgcatcgccccgtcttccctggctgcagaaaaccactaggccggtggaagtgctccgtttaattggtccctagtacgcccccgaaaggaaatggagacgatttccgtcaagttctcttagtatttctgtcatttacagaaaagcagtagctactcacatgtggctcagatgtctgtgtgggggttgtgagccagggcattaaaattgggtgttaaccgtttgccactcagaagaattattagccattggaggaggagcggtttctcttgctaagcggggttaagtgaaacttggc

SEQ ID NO: 37 - DNA, 840 bp, synthetic construct
  source 1..840: mol_type = other DNA
gtcctccctcctccagcgaaatcacacccgctttgcctcctcttgcagcaccgcgcgtgcctaggttctcggtctgcgcagctgggagtctcccccaccagcgccgcggagccgcccgcttccgctccctctttccgcgggtttgagcctcccgtggagaggaaaggtcacgaagtttcaaaggtgaaatggattttcggcggtggctggtgcgggagtcaatgggcagggcggggcaagcctggcagtcatcgacccgagcagggacccggctggccgcctccccttgcaggaaacaggtgtttgaacgcgatagcggccgccagtcaactaaggcattaaaagctcgctttataacatcgatttcctggagtgcggtggggcctgcgttctggaagctcgtttgtgaaaggcggggttgggggaattgccctgatggtaccccagttggcgcagaagatccccaaaagtccctgtcaccgcagcggcggacagcggggctgcccaggcggctgggagggcagcttttccctcccgctggggtggaaagaggagtcctgctccgttccccacccaggacaatggtcttgttaccccggcctcctcccctgcacctagggcctggcatgcagagagcgcccgcagcagagggggaagtctccaacaactggcaggtgtcccccgaggagctccgcttgatttggaagggtggttaaaccgggatccaggaggaaagcaattccgttgccacccacatcccctgggccctaccaggcttcttcccctgctagcacatctcccggatgcgacgcaggccaggtggcagggctgaagcaggca

SEQ ID NO: 38 - DNA, 730 bp, synthetic construct
  source 1..730: mol_type = other DNA
cggccgagaacggatgcagggcgggggatggctgagccgcctgatccttgcagagaacctgcaggggccctcggagggtatcccctgcgtccaagggagcgcccctccttttcagcactcgggggaactgagggcgacgtgccagccccgcactcaactttccccttccctgcaggcacagagttggccggcgggggcagaggaggagctgggtctccactgcgcccgtttaaacctggccaggggctgcgtttcctccccccaccccacgacgatcctttcttagtcttcgcttttcaacccaatcgttaatcattcggaacgcgcgggcggggagcggcgaggagggcgagctcggggttcgccgccgccgccgccgccgcgcgcgcgcgctcaggaagcggtgtggctgtcaccccctcccgggcctcctcccccctccttcctgctttgctccccctccttcctcccctcctccccgctccgccgcccgcgcccagtgtatctactccctccccacgtcactcgccagcgcgccatgcaaatcaccgccgccgccggctcccattggccgcggcgcgctcatttaatggcagcccgggcccggcgtatggctgctgggccccgcgcgccgccggccccgcgtgcgcctccgctccgagcgcacggccccgggcaggcagcgggcagcccatcccgggctcggcggccccggctctccggccctctc

SEQ ID NO: 39 - DNA, 640 bp, synthetic construct
  source 1..640: mol_type = other DNA
atttgagtttacaaagcttgtattgacagtgagcctttctccttaaaggagaggctctatttctcttaagagacatagcctctctttcaacgggagtctgcttttcttctgaaacactttcataattaagtgactttccttgcataggtgcctgaggacagctgatttttctcagaggatgaagaaagttacatattgattgctgttgctcgcggcatgttcgtaccagcgcgggcagaaacgggctcacgagcgccctcgaattcacacacactcaccgaaatacacatctcagcccgtggaagcgcattcacacacacccacgtacactcctccgcacctcattcagatgggtccacgcggagtcagggctgcgcttcccttggcatgggcgtgtgtgcggcgagggcgtgtgtgcgtgcgtgtgtgagagtgtgtgtagagaaaccctttaggcagaccgcagctacgttattctgcaagcatactgcgattcccgattccgtgtatgtaggaccacaaaaaaattaaatcgctgttaaacttttttttgtaaattctcttttccgaaggaaggcgctggaagagctactcccggtggttaaaggcccgggagacctggtggcgaatcctggggcgc

SEQ ID NO: 40 - DNA, 270 bp, synthetic construct
  source 1..270: mol_type = other DNA
gcggagcgctgggctccgctagccggactggttccccatctaactgatctaatcaatgactccgaagagccggggctcctggccccgcccccgccatcctcttctccgcctcccattggacgcgggccgagaaggcagggccttgctcctcgctcggattggctgacagggaatcagtatcaatgtttaagcggcggcgtgaggtgaatagagtcagtcagcaagagacgctggggagagagcagggatcgcgagcccggcggatgcggc

SEQ ID NO: 41 - DNA, 480 bp, synthetic construct
  source 1..480: mol_type = other DNA
gcgggatacctgtgcccgtctagccaagagtgcacccgtgtgcgcgagcgggcttctgggacgccgccgtggtcgggggcggccctgcgaggggagggggtcacagggactggccggcgccggccccgtgcgcacggaggcgggggcggggggcgggggccgcgaggggggaggcggtacgaaaagggcggcgcgcgcggcggcggcggcagctccccggcagcggcggtggagagcgcagcgcgcagcccggtgcagccctggctttcccctcgctgcgcgcccgcgccccctttcgcgtccgcaaccagaagcccagtgcggcgccaggagccggacccgcgcccgcaccgctcccgggaccgcgaccccggccgcccagagatgaccgcgaccgaagccctcctgcgcgtcctcttgctcctgctggctttcggccacagcacctatggtgagttccccggcggcccggctcgcgcc

SEQ ID NO: 42 - DNA, 610 bp, synthetic construct
  source 1..610: mol_type = other DNA
acgatccccagcagcagcagcagcagcgggtactgctggctcaggctgtagtaggtctcgtagaagaggtcttcgctggggggcggccgggggctgaagaggcgggccatgatctccccagccccgagccccggggctggctagggccgggcgccgggttacctccttcggcccggcgggccccacctgagcttttctcacccgctcaaagccgctaccaccccgcgcccccaacctcgtggcaatcccgtctcctttttcaggccctccctgcggcctcccagcccgctcccagctggcgatgaggggatccctcagtcctttcctcctcctccctcaaacccggattgtagaggtgccctagaaaagcctcatcgccaggagggcaggatttggggttggtgcgagggagccccgggttcccctgatccccgaactcactgcccgcggcgctggcgcagcggagtgggctaggtccggggagggagccggggacctgatggcggagtcacgctcgccgctgcgctctggctcagagtcccgggcgacaggcgcttccctcccggcgccgcggccccctcccacttccccgacgggacagcccggcccc

SEQ ID NO: 43 - DNA, 480 bp, synthetic construct
  source 1..480: mol_type = other DNA
cttcaacgcgtggggtaagggggggtggggaacaattactgagtgacgctaatatggggaaactgaaaagaaatgtcgattgtttttattgtaatagaaggagtgagcaaacagaaaaaccaaccccgggtgatcggaaacaggcaggcggagaattaaaagcgggtttcagacagcaacaggcccctcccctgctctcgcaaggagaaagcgggcgacgagcgctcgcagcccgtggggctgatcccacctcccgcagggctccggtcgtgtcgctgtcggaccgcagaacgccgggacgcatttccgagctcgggcccgacccaacccgctgccattctagctacctagtggagccgcggagagactcttgaaccgtgaggctccttgatgagaaggtggagaggctgccgggctattctccgagcatcttccggccgagctctgctcctctccgtgcgcttgcggcttctccttggg

SEQ ID NO: 44 - DNA, 1040 bp, synthetic construct
  source 1..1040: mol_type = other DNA
taactgagatcgggagctgtccccggcagagcgcactcacctcggtcccaggtggactgaagtccagagcggcgctgtgcagctggaagggcgcgcgatagctcaagttagaggcggccccggggcgcggcgcaggacacaagacctcaaactggtacttgcacaggtagccgttggcgcgcaggtggcatcgcatctccttccagcctgcgggctcgaccccaccggtggcctggagtaccgcgcatctccgcgcggtgcaggagcgttggggctcctccacccactgcagcgtgtcgctttcgagaccgccggggtcggaggacagccaggagaaaccccgcaaaggctcgttctccagggtgcagtgggaacgcctgcgctccagtgcgacccagaacagcaggtctttggagccccctccgggccctgggcctgcccgcaggagcgcgagcacagcgcgcagctcggcgcccgcacgcacggtgctgagcgccccacctcgcaggatgcaggcctcctcggccgcctgccgcttcatggtagcgtggtgcaggctgtagcaggcccccgaggccgagcagccagcacggtcggcagtggggtgttcgccgccgcccggcccgggccagagcgcctgccagaggaggcacagggcgaacgccggcctcattctctgaggccccgcacgcagagctgctcccaacttggatctgtcccgctcgaggacgcagagctgtgtctggagcgcggtcaccgccctctacagctggcttccccaacccggttccccctcccttcccctccgccctcgctcccgacggaccccccaactgccaagaatccccccgcgggcccagcggccaacagtttcccttaggccgcgacaggaaatgtgtgcggagcgctgccaggcccggtgcagccctgagttattctggggtcaggaacacagctggcgccgggtacaggggacgcagtcgactagaaggtcaaacaccctgcttagagcaagaggagaaattcgccactgggatgaa

SEQ ID NO: 45 - DNA, 360 bp, synthetic construct
  source 1..360: mol_type = other DNA
ggcatcaagtgcgccaagtgccaggtgggcttcagcagcagcgacctggtgatgagggcgcgggacagcgtgtaccacatcgagtgcttccgctgctccgtgtgcagccgccagctgctgcctggggacgagttctcgctgcgggagcacgagctgctctgccgcgccgaccacggcctcctgctcgagcgcgccgcggccggcagcccgcgcagccccggcccgcttcccggcgcccgcggcctgcatctgcccggtaagcgcgccggggcctgtgccggggtgagcggggtgtatgtgcgtgcgtgtgtgtaggggtacgcttgtgtccctaacgagaagttgtcagttgtgtgtggt

SEQ ID NO: 46 - DNA, 210 bp, synthetic construct
  source 1..210: mol_type = other DNA
ccggcctagggttatgcgggcgagggcgcgccccgctcccggaaccagccttggcctggcgcctgccgagccccgagccccactctcgctcggcgccgcgggccctcccggcgcttccttccccagcggtccgcggccctgggtgaggctcgaagccccgcgcgctgcccctggccctgcagcacagcagtgaggccaaccccggccaga

SEQ ID NO: 47 - DNA, 380 bp, synthetic construct
  source 1..380: mol_type = other DNA
aagatggctgcgtgggcacccggaaggtcgccgcgccaagggcccgctgagcccctcctcccattcgtccagccgcgcggcccacagaagcggaacgcgcgtcgagagcgccctgtccgctcgccccagacagatgcccggttattcattaccgcgaggcctagaggaaagagtggctgccgtcttcctgcccacagcccgccggaccctccgtcgcggctgcccggtccccggagccgcagccgccgagcccggctgtgcgtgtcgtggctgctggggagaaagaggcttccggtaagagcccccctgactgggctgcccaccccgggcaggggcggaaacggccttccccgcgtggggaacaatggcgttattttgga

SEQ ID NO: 48 - DNA, 464 bp, synthetic construct
  source 1..464: mol_type = other DNA
cgagaacttttttctccgcgtcttcgctgtcggcggcggtggctaaagaggcggcggccgcggtgcccgtggctgcgggggccccgtccccacccgggtttcctgcgacgagggcggcgggcgccggggccgcggcttggggtgcaccgctgcccacccggctcttggtcgcggggtcctggggagccgcggcggccgccaccgcaggagcctgctggagggtggtggtgatgatggtggcctcgcccgcctcgctcatgcctcctcctcctctgctctcgctcatgcgcctcggtggcggttggtcggcggttagtgcggctggtggtcgccgcggccggggctcgctctcggggaggccggggctgatctcgcggcgcaggcggcggcggcagaggtggggtcgcgcagcggaggcagctcgagcttcgggatgcgcgctcgcttcttgggctcctcgctcg

SEQ ID NO: 49 - DNA, 330 bp, synthetic construct
  source 1..330: mol_type = other DNA
gtgttcccgggaatatgcaaatgccagggcgcaggcctcccaaatcagaccgcgaaggctcggggctgcgagccgaatgcccgccgagctgcctgggaacgggaaccgcgcacccgggaacgccaggcgtttctcttcatccaggaacggcgcggagagcctccggctggggagctaaaccgctggggactccgcggctgctgcctgagtcgctgtccgctgcccgcatcccttccgccctgggcctctgcacggtctgcggttttctgtgcgcacttggtcttcagtactagcacccaattacgtctgggtttttcttctttacagagc

SEQ ID NO: 50 - DNA, 630 bp, synthetic construct
  source 1..630: mol_type = other DNA
gcctaccttggcacttagggaccagaagcctctggatgtctagcaacaggggtcacgggatcactgcgtggggtctctgtaagcagtcccctgaggcagtgcaaaaccggaaacctgctctgtgcggggccgaatcagttcatgggatttgggagtcaggagagacgtctttctcctcccagctcctacactccgggtaccaaggcccgaaatgccgttcccccagcccgggtgcggggtctgcagcaaggcctcctgatttgcaaacccctccgggctgcttattcctggtgccatgcgcagcccttgggacctagaagattgtgggggagggaaggtcgcacgtgcggttttggcaccgaccgtgcctcctagtccacctgtcccccacagctagctgccaactcggacggaagcgcggaacagcgcaatgcaaaccgcccaaagtgaaaggaatgtaattgcgctcccatgacagagccagacgcggatgcagtttaggaagggcgccgcctaccggcccctgggagccatgcgattcgaagggagggggacctagaggagacccccgctatccccccacccaccgtggggccttagctttgaactccggcccggacaaacttaaac

SEQ ID NO: 51 - DNA, 360 bp, synthetic construct
  source 1..360: mol_type = other DNA
aaaccggccccgacgccccgcagaggcccgcctgaagccagcccggcgccctagcagatgcacgtgtctgtcgaatcgctgcctccgagcccacccccgagctcgcatgctgtcacccattccagcctaaatgtgaccataaaattagggctgctgcttttatcgagaacacctgcttcctctcgttgccttgtgtgaaagtcgcgttgtattttcctgcgcttggcgctgcgcccgcggagctcagggccgtgacccggtgctcgcagccccccgaccccgcagcggagcggcggcttacagacctggggccacctggagacgccgggatgggaccgcgagggcagctccgcgcggagg

SEQ ID NO: 52 - DNA, 800 bp, synthetic construct
  source 1..800: mol_type = other DNA
ccgagctccacagcctttttctggacaacagtaccctagtgggcagcgtgggtcccggctcagtgctcgagctactggaggcctgcccgcgcctgcgcgctctcggcctgcacctagccagtttgtcgcacgccatcctcgaagcactggcggcgccagaccgagcgcctttcgcgctcttggctctgcggtgcgcgtgccccgaagatgcacgcgcgtccccgctgcccaacgaagcctgggtcgcgttgcgccgccgccaccctgggctggcagtggagctggagctggagcccgcgctgcccgctgagagcgtgacgcgcgtcctgcagccagccgtccccgtggctgcgctgcgcctcaacctctcaggcgacaccgtaggcccagtgcgcttcgcagcacaccactacgccgcaaccctgtgcgcgctcgaggtgcgcgcagccgcttcggccgagctgaacgccgcgctggaggagctggcggcgcgctgcgcggccctgcgcgaggtgcattgtttctgcgtggtgagccactcggtgctggacgccttccgcgcgcactgcccgcgcctgcgcacctataccctcaagctcacgcgcgagccgcatccctggaggcctacgctcgtggcgtgattgggcgacttctctcccccgtccccgtggacgtaagcgctctgagagggaacggggggggtgtccaggcgcgccccgagtgctagtgccttcttttgggattgttgccccccgggtctttaccgagttgggaactgtgatggcatcgggaccagtcct

SEQ ID NO: 53 - DNA, 420 bp, synthetic construct
  source 1..420: mol_type = other DNA
cccgcggctgctctagctgcctcgggccgggccgagaccccgcagaagcctccctacagctacatcgcgctcatcgccatggcgatccaggacgcgcccgagcagagggtcacgctcaacggcatctaccagttcatcatggaccgcttccccttctaccacgacaaccggcagggctggcagaacagcatccgccacaacctctcgctcaacgactgcttcgtcaaggtgccccgcgagaaagggcggccgggcaagggcagctactggacgctggacccccgctgcctggacatgtttgagaacggcaactaccggcgccggaagaggaagcccaagccgggccccggggccccggaggccaagaggccccgcgccgagacgcaccagcgcagcgcggaggcgcagccggaggcgggg

SEQ ID NO: 54 - DNA, 330 bp, synthetic construct
  source 1..330: mol_type = other DNA
taaaacaagtcgctgagcagctcggagcctgttaaccagcgcataatgccccatgttaaccatcctgggggatgtcagggcggccacgctgatgcactgcggtcgggcgcagcggcacagccttcagagccgccgccgctcggttccttttcccatgatgttttggactccgaatgccgtttctcgccgccggcctccgtccctcctctcctcctcctccaggcccccttttgtgggcagaattccaaatgccagcggccgctgctagcaccggggagaggaggcagcgcgcggcgcggagaggcgaggaccggtatctggagactgggg

SEQ ID NO: 55 - DNA, 1080 bp, synthetic construct
  source 1..1080: mol_type = other DNA
tgtattcctccattgtttgaaattttcctttataattttttaaaatcattacatgtattactttttcatggaaaagcttctcctaagtaaagtactttggaggctgtaaaatgggagaggtgccatccagagagcgaatatcggggaaagctgcgatcggtatttactcagcaccacccatatgtgccaggcttgctagaccaggagctacagagcctcagtgagcctttgggtttgaagaaggggaagaaagttaactggcagaggggacaggggtgagaaaggagggacctgccgctagagggagcgaagagcagggaggcagtttgggacacttggcaggcctggagctgggtttggcagggcccccgggaagatggaggaaagcagtgggagctagggttgcctgtacggtgcaggatttgaatgccagaccgaggagccgcagtactaggaaggtccaagcagcgtgcccaatcgctcctccggactacagaggagcccgggaacccagggaagagctccagtgattggtccccctgcctgtcagtctggtgggaggcccagctctctaccctccccgcccgagagtcacttacaaccggtagagaatggtggtctttccttcattgtccagtcccacgatgatgaccgtgtgctctggaagcgtcggaggaagggcagcgtcagggccaaacccgaagaccaagggactgtgtgcccccaccagacctcccaggaaggagatagcgctccccgcaggctggggacctctgcccaccgtaccgcgtctgcttcccacccagctccgacacctgccccactccgggacggtggaaaagacagctgagcccccacctcccttcacattccagaaaagtgtctgaaaggcccggggcgcttcggggcttgccaagagacggtgtttagagaaagagcataacgcgaagtcacaatcgcaggaaactcgcagcagccccccatccccgccgctggctccgtttagcggggagaaaggagggtcgcccagctttgcgtcctggggcgcaccgaagcgccgggacccaagaggagcaggca

SEQ ID NO: 56 - DNA, 440 bp, synthetic construct
  source 1..440: mol_type = other DNA
ccttcccttcccacatccacccggactctccttccatctcgcgtcctcagcgtccgttgggctccactttaaaaataaaaaaatcaaactaactaaaacgaaggtgggaccccccaggctccccttcttccctggcctctggaaaatccagctgggaggacgcaggggcgggggcagatcccggctgggccggcgcctcctgcagcggctgtcatggcggccacagcttcttccctcgccccccccccggcccgccgcccggccccagcgccattttgagcatcgtctttgcttccctgtctccctcccgctctcctcgctcctccttctctccccgcaggtccagcgcctcgcgacgctgccgggccaccctgggcttctcgcccggctgctgcttggatggtctcccaaaccccagcagcccgagtccatgggtgtcc

SEQ ID NO: 57 - DNA, 280 bp, synthetic construct
  source 1..280: mol_type = other DNA
gaaatacaataaaacttcaccgtgttgatgaacttcaaacggtttacatcccctcctgtcctgaaaagaaagacgtccgggggcggattaaaaggaaggggaaacaaatttacgagcggcagaagcacccgcctcggcccctttctttgcccgcacactttgtccgcgtggagagaagcagccccagcccccgggacttggcctccgccgaaccggccgccaggcgcgctcttctctcggaggagtcggcgctgacccagcccgcgcccggcagagactg

SEQ ID NO: 58 - DNA, 810 bp, synthetic construct
  source 1..810: mol_type = other DNA
aaaaatgtacatgcagtttcagataatttatggacttcctgaaactcatcctctgaaccctgttccccaaatcaaacatttaacttaaaaaaaaaaaaaagaccgagtcacttccccaataatttcctcttcaccgcacccctcagcttttggaatgaagctatgtcaaaaagacctgcctgggtgaacccctgcaaatggaggtcagctggacctcagtaaaagcccagtgggaaggagcggggacgattctggcgatcacatgaggaagggagcagcctcagcagcgcaggtggacacagtcgtcaaccactcgctggccgcctgcgacgtgaagcacctgcgctgggcgcggcgggtaagcgccgacactagcgggcgcctgaacagtccagcggcgtcagcgctgcggagcaggaaagggtcccaaacgcagggcggccatgcagcgtggcgcccacctgtccctctcctcgaagcgctgagccagcacgatgcacataagggaaggaggaaggtggcttgagggcagtgaccgagcggacagcggaagcccatttcaggagccccggtacccgagctcgggcccctttccaatcccccaggcgtccttcccatgtccttgtctcctagtcctgggaatccacggccactccactctcctgcagtaccgcggacaagaacaccatgtactcttcacaggcaggtgtcttctggtggacccaactctgttggtacttgtcgtctccaaaaagtccccaaatgcgctagaggccagccagcccggtatccgagaggtaagagccccgg

SEQ ID NO: 59 - DNA, 370 bp, synthetic construct
  source 1..370: mol_type = other DNA
gcacgtccactgccctttccttgacggccctgggccgagtccctggtgccctccggctcagaggtgctgccgcgtgcggtcaccgcacctctgggctcaaagcggtttgctccatcttgatgggccgggccgctccaccggggctgccgtcggggtcggccgggggcgcgggctgcgagcgcgctgccgcctcggccctgcgtccggatccccggcgggcgccgtcgtctgggacccggtgcaccggccgtttgcgaggcccgggaggggtggggcctgcggccagagctgcggcttggtcgcgcagcagccgcaccaggaagccgatgtacttcatggctaggcggagcacctcgttcttgctcagctt

SEQ ID NO: 60 - DNA, 910 bp, synthetic construct
  source 1..910: mol_type = other DNA
aaacctggcattggtgggggcggggttactgcgatgtgggcggagcttgtctgggaggccgggtcccgtgactccgcccaatctcccgcgtaccctagacgtgaacgagtgcctggagggcgatttctgcttccctcacggcgagtgcctcaacactgacggctcctttgcctgtacttgtgcccctggctaccgacccggaccccgcggagcctcttgcctcggttcgtacccgggctgatcctggccccggaaagggtgggcttagggcaggaaaaggcgggacggggagaagagggcgaaaaggggaaaacgagtttttagccggggtattccagcaggatcagggggcagctggtgggagtctcgaggcagtgagggggggcggggcgtggagatgaaagggccgagtctgggtatttggaccgtgattgtaaagaagctgttctaaacccgtcggggggcggtgtttgcagggagggaagtagcgtgaggcaggttggggaaggcgtgagaggcctaggagagccgaggggcggtggaggggtgtggcctagaatgttaggcggagcgggaggtgggccgggccttcggacgccctgtcccgcagacgttgacgagtgcagcgaggaggacctttgccagagcggcatctgtaccaacaccgacggctccttcgagtgcatctgtcctccgggacaccgcgctggcccggacctcgcctcctgcctcggtgagaggccccgccccggcctgatccctcctcccttcgactccccgactcgccgattggcctcccacctctgtctttcctcctccgcttctcccctccccttacctctttcccccgcctcctctcctagcctccccaactctcctctaccccaatcttctcctgccctctcctctg

SEQ ID NO: 61 - DNA, 360 bp, synthetic construct
  source 1..360: mol_type = other DNA
ctgccccaggccccagggacgcggctggaacttgggacaggacttcgcagacaaagacggtccagtcgaagctattcaatatggccgaggcgctgggaggccccagcccgggcctccagtagggtcccccgcccccaggcaggccgccacccagctccaggctgaccttgacgcgccgcagcctgccagcccgcgcgcttagagctctgcaccgtggagcgcgcgggcaggaagttcctcctcgccctccgccagaatgaggcccaccttcccccgtctcatcttacactcccagactctggaagactgacaggggggctgtccactcccttcggggcggcctccttcccctcccccaca

SEQ ID NO: 62 - DNA, 810 bp, synthetic construct
  source 1..810: mol_type = other DNA
cccgagagcgccacgaagagccctgctcgcctctgcaggtgcccggcgtccacacacacaccccctccgggggcaacccgggcccccaatcctggtccacttggcgcgtccccatctcggccctgcgctgctgcctcgggcgcccacttctctttcctcctcccatccgggcttcctccgtcgggatcgctggcgcccccggcctgcgagggcccgagaggggaacgtgacgctggcaaaacgcggcccgggcgcgctgtgaagcgaggagattgcgtctgcggaggacaggaagaggggaggggagggcgggggccacaggcgggcgggtgccctgcggggcctgctcgcagcctgggcggcttcttggggggccactagcggcatttagggggcagcctccggccccgacgggccttcttcccgcgcgcggccttggatccagtgggcgcctggcccttggcctcgcctgccctggcatgggccccggcgcccgcgctccccttcttcttcctgcttcctgcggcgtcgtcctgggccgagccccggctcccggctttcttctcgggagtgacgccccggcctcggcctcggccgcccgcgcgggttttgcgggccccggaagcggtgggaggcgcgccggccggagtcaggcccctgggggccgtgcgcgccctcttggcccggggcttcctggatgccctgtcctccggctccgacgcctcgctctcggtgtcctccgactcctcctcggactgttcgtccgaagcctcctccgacccctcgttggaccccatgttctcccgggcg

SEQ ID NO: 63 - DNA, 320 bp, synthetic construct
  source 1..320: mol_type = other DNA
gcggcagtcgtggagaccacagggagccgggattagcccgaagtgagggtctgtctggtagcaacacccaatggcaggctgtgattgccatgttttttcagactccgactggattccttagggaaatcccgtaccctttgaaagcaggatgcggccacgccccccgggtggcaacattgtgattggtgggtggggttcaggaggtgtcgggcctgcccccgctagagctagaatgctgttctgattggaccatcgccgggtgggcgtgacatgattgtccgggctggggcatccgccgaccggcctgaccgcgcccgggc

SEQ ID NO: 64 - DNA, 520 bp, synthetic construct
  source 1..520: mol_type = other DNA
ggttgcagatttagatctcaaggctgaaaaggataagcttccaccagagcatcctgtagcgcctcctgtcctgccctgccctgccctgcgcgcgcaccgcactcacacgtacacccggtcctcgcacgcgcacacacgcacactgttccccgccgctctaccctctagactgcaagggaaacggctcctctggggcgcgctcaccggaggtgaggcgacccagcccggcccaggccagcctggtacccagcgctggtcgctgcccctgtggggaggcagcgcggccttcggggctccagagcgcgcgggcccggaacgaggcgcgcggccgctggcacatgcggggactgcccagcgcggactggagaaggggagcgaaggggtggggagggggtgacgccggctgcccaccccgctccgcgcctccctctgtggccctctctcccttcttcccctcccaccctgccgctcccagccgcgtatggaactcgcgtttggaaaaggcacggagcaggcgggc

SEQ ID NO: 65 - DNA, 280 bp, synthetic construct
  source 1..280: mol_type = other DNA
gtgcgcgccggtggcctcggacgcagccagcgcgaggacctcggcgcggcggtagcgccccggcgggccctccaagtgccggcctgcagggggcgccggcgcgctcgcgccccgcgggttctccaccgccccgccttcgcccaccgaggaagggtcggagggaggacccggacgggagcaggttcccttcgcgcctctctggagggtcggcggggccctcaggctggtgctcacccgtaggaccgggacggtgttgtcacacttgtcaccaacgacggcc

SEQ ID NO: 66 - DNA, 450 bp, synthetic construct
  source 1..450: mol_type = other DNA
gaacccagcttgtcctaaccgctttcgctgcgggcagcgctggccacgcggcccccgccgccggcggttctccgtggccaagcatccttggccttggagcccaggggctgcgttccccttggggccggggcgggagagaggacctcggtggtactcgcccgtgcgctgggcgcagccgcttggccctcagccctctggcgcggcgcccacccgctgggtcccgccccggcagcgacgcagggataacccgcggccgcgcctgcccgctcgcacccctctcccgcgcccggttctccctcgcagcacctcgaagtgcgcccctcgccctcctgctcgcgccccgccgccatggctgcctcccccgcgcggcctgctgtcctggccctgaccgggctggcgctgctcctgctcctgtgctggggcccaggtgagcggggcgatgccaggctg

SEQ ID NO: 67 - DNA, 520 bp, synthetic construct
  source 1..520: mol_type = other DNA
cgcttccccgagagcacatcgagggcggcggcccgagcgacccctcagagtttggcaccgagttcagggcaccgccaaggtctgcggcggcctctgaagatgcccggcagccggcaaagcccccctactcgtacatcgcgctcatcaccatggccatcctgcaaagcccgcacaagcgcctcacgctcagcggcatctgcgccttcattagtggccgcttcccctactaccgccgcaagttccccgcctggcagaacagcatccgccacaacctctcgctgaacgactgcttcgtcaagatcccccgcgagccgggccacccaggcaagggcacctactggagcctggaccccgcctcccaggacatgttcgacaatggcagctttctccggcgtaggaagcgtttcaagcgccaccaactgaccccgggagcccacctgccccaccccttccctctacctgctgcacacgccgccctgcacaacccccgcccaggccctctgcttggggcccctgccct

SEQ ID NO: 68 - DNA, 230 bp, synthetic construct
  source 1..230: mol_type = other DNA
caccctgtcgccaacccagggcccatccgacgccggggcagagcccacggcgtcggagggccccggtcgggtttccgagggaaggcctgccctgccgccgcagctttccggccattgtccccgccacccgccccgcccctggccacctaccccgtggctggcggcgggctctgccggtgctggcgccgccggtacagccccgacgcggccagcgcacccagccgggccgc

SEQ ID NO: 69 - DNA, 500 bp, synthetic construct
  source 1..500: mol_type = other DNA
aaccgggaaggaagaaaatagatcagggagctggagaggagaaagaactgctctctggagacaggagggtctgaaatgaaaacgcccaaaggatgtaatttttttaaaggaaaaagggagatttgttcgaagggacacagctgagtgattctggctgaacaatgaggcctcactttcctggctacatttcaccgactaaaattctctagccttatcgggccagaaaatacggatgtccccgggcagaggttggagaggcgggggaagattaacgggcggcttattaaagagccatccgtcagctcctgcgcgcgggagatagcggcagagcaggcacgggacacgcccgcccgccctagccccggagcgccgagagccgcccgccgcctgggtgctctctgcacctgatcttcccagcctccctgggtcccggggcgagggcggtggcagtttgcagtcagagcagagtggccgctttggggaactactcccttccctgc

SEQ ID NO: 70 - DNA, 290 bp, synthetic construct
  source 1..290: mol_type = other DNA
ggggctctgtcctctagaccctgggactcggcgtttagggaggaacaggcgctccattcctagacccagccattttccaccgaaagcccagccctgccgccgggcggcctgggagtttggccttctccccagcgcaggacgcccgtggggcaaggccagggctgcatccctgtgctggggcagccggcggccgcggatctttgttctataaatagccccgtcccactcccacccccacctccacccccaccctgccgccattctgggcttaatggcccgtttcgcgccag

SEQ ID NO: 71 - DNA, 1070 bp, synthetic construct
  source 1..1070: mol_type = other DNA
ggggctggtcctcctcccttccaccccaagcccttttttgggttacacccctgacctgctattctctgctttccagggctgtgggcagccccttggttatggaccctaccagcatctgcaggaaggcacggcggctggccgggcggcaggccgagttgtgccaggctgagccggaagtggtggcagagctagctcggggcgcccggctcggggtgcgagagtgccagttccagttccgcttccgccgctggaattgctccagccacagcaaggcctttggacgcatcctgcaacagggtcagtgtggggagggggcggaagtggggctgctttctccctgctgtgggacccgaggagaggagaactggttcgctgaagttgcctgagccccacttccccctcacatgtgtctgggcaccctgcaaggaccctgcctcccaggcccctggggcagccctcccgccgcaggtttcaggtcccaggccccagctgaccgccccagcccgcgctgattgcacctgtctgcattcacagacattcgggagacggccttcgtgttcgccatcactgcggccggcgccagccacgccgtcacgcaggcctgttctatgggcgagctgctgcagtgcggctgccaggcgccccgcgggcgggcccctccccggccctccggcctgcccggcacccccggaccccctggccccgcgggctccccggaaggcagcgccgcctgggagtggggaggctgcggcgacgacgtggacttcggggacgagaagtcgaggctctttatggacgcgcggcacaagcggggacgcggagacatccgcgcgttggtgcaactgcacaacaacgaggcgggcaggctggtgcgtacgggcaggatggagtgagtgtgtgcggaaatgtgagtgtgcgcgcaggagtgtgcttgaggaagtgttggcaggagtgagggtgtgtaggtggaggggggcgttaatgtgtgggggagtgcgcacgggcggaaagatgggctgcaagcatggatggacatgtgggaggagaggtgtccagcctccaagaaggag

SEQ ID NO: 72 - DNA, 790 bp, synthetic construct
  source 1..790: mol_type = other DNA
gagactttctccactgcagggcggcctggggcgggcatctgccaggcgagggagctgccctgccgccgagattgtggggaaacggcgtggaagacaccccatcggagggcacccaatctgcctctgcactcgattccatcctgcaacccaggagaaaccatttccgagttccagccgcagaggcacccgcggagttgccaaaagagactcccgcgaggtcgctcggaaccttgaccctgacacctggacgcgaggtctttcaggaccagtctcggctcggtagcctggtccccgaccaccgcgaccaggagttccttcttcccttcctgctcaccagccggccgccggcagcggctccaggaaggagcaccaacccgcgctgggggcggaggttcaggcggcaggaatggagaggctgatcctcctctagccccggcgcattcacttaggtgcgggagccctgaggttcagcctgactttcccgactccgccgggcgcttggtgggctcctgggcttctgggctcacccttacacctgtgtactaaagggctgctaccctcccgaggtgtacgtccgccgcctcggcgctcatcggggtgttttttcaccctctcgcggtgcacgctttttctctcacgtcagctcacatctttcagtacacagccactgggtctccctgcccctccagcctttcctaggcagctttgagggcccagacgactgaagtcttactgctaggatgggaacacgatgaaaaaggaaggggcccagtcaaaagtcctctcctct

SEQ ID NO: 73 - DNA, 530 bp, synthetic construct
  source 1..530: mol_type = other DNA
gatgagggaaggacgcagaaaccagggcggaggagccgcgaggggcaggacgaggctgcatgggccagcgagggggtcgacaccgagccagagtgagcgcggggcctggggcgcagagcccgcccagggagccgggagacgccgcgcaagctccccggacaaacgcaatgaccgaggacgcgcgggcgaggccgtccagggagccctggtccctcagctgcaccggactgagccgcgaccgctcagcacgcgctgcttataaatcaggggtgcgcttcccaagccccgggtgaggtcccctacgtcggcacagccttaggagctgcaaagcagcgcgcgcctccggggctcctgcgcgccccttgaaccccgcctcccgcatcctcctgcaacagcctggagctccctgtgcaggacgcagcggggggcggggggcggtcttaggaggctgcggggcgcactcccacctcctgcctccccgagacccccagcgccttctccagggtttagagcggaggtgaaggggcctc

SEQ ID NO: 74 - DNA, 270 bp, synthetic construct
  source 1..270: mol_type = other DNA
ctctcaggctcggccgcccgagggagtttcttttattcccagttcggctttcttttgcgaaggccgagatctgggcctgccaggggcctgcccgagtcctctatcgcgggtccacgtggccaccaatgacccgcggcgcccccgcgtgtccccgcagccactccgcggaagcagcggcgggagcgcaccaccttcacgcgttcacagctggacgtgctcgaggcgctcttcgccaagactcgctaccctgacatcttcatgcgggaggag

SEQ ID NO: 75 - DNA, 1114 bp, synthetic construct
  source 1..1114: mol_type = other DNA
gtgatctaagtcacacaaacaaaaacaagcttggtggcagcttgttgttgttgagtgttgttgagttgatttcttgagataagcagtgtaaagacaaaagggggagcgatgcaggtctgtttgttttctttccccgccgccccactgtcccttttctcgtctccccgcgcacctccccagacgccctgccggggtggctccgcggatgaggcggtcatttgctgtccgctttgcggggacgggtcactttccgcgctggcgtgaaagcaaatgtggagctgcttttggaaggcgccggccggacgtcggctaggctccttctccccgcgggctgggggccctggggctctgcaaggctctggctccgaacagattgcgcctcccgcctggctgccagtaggaactggggtgggagccgcgtaactaacagttgcgcgcaggaggcgagccccaggtgtgagcgcagaggctctctccccagcccgcgggtctgggaacctttcaggacgcctccctccccaactcctacccatgcgtctgctccctaggccgagcccccctcgtgaggttttaatgaccgcggacgcaggggagcccgcacttgagcgaggaccgacttctctggcgggtccacgctgctcgcgcttgcgtccgcgggtagcgcgctgtgcccgggtcagggggcgagctgcgagaagtaggaggggtcaagacccccagaaatccctccatgggcacacacacaatcaagaatagggttgagggtcttgagaggtagaactaccctaggcagggcttctccaactcggcctttggaccccgcgcgcgcccaagggcgtgcccaccgcggaagcacagatcatcttcccgggactgggtctcctggaccctgcgttgctccctttttcctagcggccccgtagctggctgccgcatgtagggcgatcttcattaacttggacgcccaacgtgattgaaatagagaggaggaaaaaacacatttgatctgggccgaccctttgtcttgaagcagattaggcagcctagaaataggaggaaaaacagaaagtctaggcaggaaaacttattgtgttcacaacatataaaaatgatc

SEQ ID NO: 76 - DNA, 680 bp, synthetic construct
  source 1..680: mol_type = other DNA
gccgcttcctgagccatcagtcccagcgggtacgttatcgagtagcacaaacagttggatttttccctcaagaaccgagtctggacgcggagatggagccaagtgtggctgcattttcggacccggaaatccgttgggcactgaaggacttttcgaaccctgtagcgctgttgcttcgcggtccatcgtcgccgctgcagacggatgcgctccccggcggctctacgccctccagtcccggccaggcctctgggctgggagccgagccgtctcgggccctccggcgccgcgttttctagagaaccgggtctcagcgatgctcatttcagccccgtcttaatgcaacaaacgaaaccccacacgaacgaaaaggaacatgtctgcgctctctgcgcagcgcttgggcggcgcggtcccggcgcgcggggaagcggcgtctccgctaaccgaggcgctggaaggggaaaagcgaatgcggaatcgtccaggactccgaaggtcggggccgctcgcgagcaccgaaggggaggagccgacgaagaccaggagtgggccgcatttcggtactgtttccccgagatcaggaactttccgggtctaggagcaacgcctggagggggctgtagagacccagccccccgggacccgcaactacaatgggccggagcttctaaggtcgc

SEQ ID NO: 77 - DNA, 440 bp, synthetic construct
  source 1..440: mol_type = other DNA
tgcgcgccagactcttgagctgattgtatccgatgtcgagaaacttgaggctgcggcagtcctggaagatgcgcacgggcacaaactggatggcgttggcccgcatatgcagcgtggtgagcttccgcagcccgtggaagaggtcgggcgcgagcgcctgcagcttgttgtacgagaggtccacgctgcgcaggttgggcatgggccggaaggtggtgttgggcagttgggtgatctggttggaactcagcgtgagttccttaactcggcgcagtttctgaaaggcgtccccctgcacggagcagatgtgattgtgatccagatagagccacgtgagctgcattaaccccgtgaactggccggcgcgcagctccgagaggctgttgtagcgcagggacaagcccagcaggccggacaggttgtggggcgcctcggtgagg

SEQ ID NO: 78 - DNA, 470 bp, synthetic construct
  source 1..470: mol_type = other DNA
cggctccagtgtaagtgcagcgtgccgtgtgcggcgtcccagcccgcaccccgcaccccaccatgtccctctgggtgcggggtccgcatgcccgcgccctccccgcagctgccggcgtccccattaatcacgcggcgctgacggcgctgatctgcgtgctggcggcgtgacggtgacggtgacggcgggtgacggagcgggcaatcggcaggcgcccatcggcccatcttatcgcggcggcccagccgccgcccgctgcgcgccgcccattgatcgcgccgggcccctgcccgggggtcagcgcgggccgggccggcctgacagccgcatccgtcgccgagacgcgcgctgatggccgcccagcccgcacggcctggccattaatcaccgtgcctgccgccaagtgacgggaggggcggcagggcagcaggggacgacaggcagcccccgtggccggcccaccccttcac

SEQ ID NO: 79 - DNA, 700 bp, synthetic construct
  source 1..700: mol_type = other DNA
ctccgtgtggatctcggtctttgtctcgccctctagctctctcgtgcgcctgtgtttaatcgtgcgcttctctcaatctctctgtccctccctttgccccctacacgcgtgttctcgtcaacagatctccagaggtggtgcagaaacgaccccgcgccggcgccccatcctgcggccagtgcctccgcgccccggctccggtccccaccgtccccgccccagatttccggaggagcaggcgggcggggtcccgcggggccggctgccgtcagcgccccttcccggcggccgcgacccctccccgctgacctcactcgagccgccgcctggcgcagatataagcggcggcccatctgaagagggctcggcaggcgcccggggtcctcagcgctgcagactcctgacctgccgactgcggatcccgagtccccggatcccggacccatcctgtggagcccactcctggcaggtaaccgccccaacccctctccttccgcagacggtgtccgggagcactggaaagggagccctctcggcccgccctcaccttcggttctctgagagcttcctggagaacagggccggtgcacaggcgggcgctgtgctcgcacccccgggtcctccccgagcgcaggccggggacccgacgggaggcacggcgcgggcacatcggaggctctcgctgctgggcactcaggtc

SEQ ID NO: 80 - DNA, 560 bp, synthetic construct
  source 1..560: mol_type = other DNA
gaacctgggagttcgaatcccaatctcaacgctagaaatagaattttagcgttgaagttaggattttagcgttagaatcagattcctggcttagtcatgaaagcttagcattagaaatagaatcttagctttgcaaatagactttgagctttagaacctgaatcttagcgatagaaatgggatcttcgcgttggaatgaggacgtcggccacggagcttggagttagaacgtggcgatggggacctttgggggagtcttggcggggacgcaaccgttagcgtcgcggctgtgcctggcgctgcaatctcaatggttgcactcccggcgaggcagtcccagcgttggcttccccagttccgcgtcacattgaagggtggcggtggcgctggcgatggggttcctacggcctcctgccttgcgcaagcagaactgggcgcaggcactacaggcacccgtgggggcgaggacgcctagaggtgacaggtgtggacgcagagccggggcgaacccgccgctgctcccgcccctgcacgaggggcagtggtcctgcttctgggag

SEQ ID NO: 81 - DNA, 220 bp, synthetic construct
  source 1..220: mol_type = other DNA
aggtgtaagcccaaggctgctgcctgcaaaggcaatgccgtggagactgggttccacagcgacctgggtttttaagtcaccgaaagctacagggcagggtcacaaacactctcccgccttctctgcagaaccgtgcggctgacaggagagcgttgcgcagaaaattcgaggccgggcgctggaggagtctccgcggcccggagaaggcacagaggcgccc

SEQ ID NO: 82 - DNA, 260 bp, synthetic construct
  source 1..260: mol_type = other DNA
gccgcccctccctcgggattccacagggccctgacccggcctttatctctgcacggtcagcagtcgcgggggcttcaggaaggaggacaaaggcccggtgtcaccgcgggcgggggctcggtttcctggctctctgctcacactcacagcctttagcggttgttgggggaagatttttaaaaatatgtgtcgaatttcctttttttctcttctagaaacaaacaaacaaaaaaggcaaaaggcgaattccccttcactcc

SEQ ID NO: 83 - DNA, 300 bp, synthetic construct
  source 1..300: mol_type = other DNA
cgcccggcagtcgagagcaccctgtgcccagactggccgactcattctcccccgaattttgtttagagctggcaagggggacttagctcgcgccccaagacctgggcttgcagcgccgccaacaggcccggggacacgaggcgctccaggccggggtcttcccggctgctggcccctctcgctccccacccgctggcggcgcctcggtcgcccgcaattgacccaacccgcttcctgcgtttgcccctcaggtttcccgtttctccacaaaggcctaggggagcctcgcccacaggctga

SEQ ID NO: 84 - DNA, 307 bp, synthetic construct
  source 1..307: mol_type = other DNA
gattttgttgcgcgacagcctcagggttttcagttttatcaggtcactgaaggtgccgggggcaacggcggaaatgtggctgtcggagatcatgaggcgctgcaggacggtcatgccgctgaagctctggctctgcaggacgccgcggcccattccgaagagcaggatgtgcgtgaggttggtgggcaggcctagcgcggagatgcgcgccacgtcgccccccgagcactgcgcggcgtcccggaagacacacttgcaagctggcggacaggggaagggctgggcgcgcagaagcccgagcaccgcg

SEQ ID NO: 85 - DNA, 710 bp, synthetic construct
  source 1..710: mol_type = other DNA
cgcaaataattcacctggataggtaaaggcgagcaaacactgtctgcttgtgaatccctgacaacttccctaccttcctcccgagggagaggcagagcctctagctccatttcagacctgcaaggaaagggctttgagaagggaactggggagaagcacgttccaggtgtgggttcagctcggcactcgccacaggcctccgcaggtggctctccttggcagcgaggcaaggcgcaaacccgctggcttgggaagcctgacgtgacccccgcccgccaagcctggcaggaagcgacgccggggttctccgcaagaagaaggcggcctccgggtgtcggccgcagccaggctactgtgctccggggcaaaccgctgcaaagtcctagtgaggcagaactcgacccccaacactcagcagcctgccgtccacccatccacacccccgtctcgccccctgccccaagctggcagatgccttgtcgcccctctaagaccgcacccggattgggtggctgcaaagacgctcgccaaagcgctcagagccccagggaagccgtggcgcctggcagctccctctcctctgggagatctgggggcgccagggctgccggggcctagcacagcgtgcgccacgaggcttaaggatgcgaaaactactgggaggccgagagaggtcccgggagcaggggcttgaggaggcaccttggggc

SEQ ID NO: 86 - DNA, 530 bp, synthetic construct
  source 1..530: mol_type = other DNA
ccagttcacactgcaactccccctttagttaattgctgagtattctgctttttctcgcgtatcctcagaaccaaaggtgtcgatatttggactacagagccaagcagttgcaggacgacgtgcaacctcaaaggaaagtgtcccgaaggccgcttcctttttgggggttgtggatctcagagccaggcgctcgcggggttcaggctgctgagaccctgccaagggttaggtttatttaccccacgaacattcgcccagcgtccactgagcgggccctgcctcactgcctcccagcgcttgccgggcccgctcgctgatcggcgtccaccttcccaccgttcctctgcttcctcaggagattggagagcgaatggtgcctctaacaataaaagtggtaagctgcgactttgtcagccaggcacgtttttaagtgcttttaaccatacattaactcattaaaaaggaaaaccatcatcgccctcattttatagatgaggaaacaggttaagccatttgtccaagatcaggat

SEQ ID NO: 87 - DNA, 520 bp, synthetic construct
  source 1..520: mol_type = other DNA
cttgtcgggtgtctccaaccgcgttgccgcctggctttcagccgtgcgctggccagccgggcgggtgacgtcacctctccccgggtccgtgcacccaggccttccctgctccgggtgagtattttttcatgggattcagccatctgggggcgccatttcccaaaacagagattgtccctttgaggttgcaagtcaggttcctttacgctgagatcccttacatttttacttttaggaaggttttttcgaggggtggggggatgacctcattccttcgcctttggccctccctctacccggccccacaacgaggacgcccctgcagctggctgggccgcttcctcttgccacgcggggggacctgtgcccgcggccgtgcacctccgtgtacgcttctgctcctcggaccccatcggttcagacgcggatttccgagaacgagaccagagtctgcgtctggagagcctttgcccctcatcttaggaagagtctagtctgagaacaaacagtttgccaactc

SEQ ID NO: 88 - DNA, 1053 bp, synthetic construct
  source 1..1053: mol_type = other DNA
aaaacgcggtgggacaaggctattgagtctatagctaattctttcatgtatataaaatgtatacatatgtatatatttttatatacataaaagaattcatatatatgtatatagctatgtggagccctgaagcaattctccatgcttttgtctccctcaagttccccaggtggaggcagtcataagcattataagccgccttagtgaccaccagggacggaaaccgttaattatcacgtttcctttcatctccaggggccctttggcccgtgacacaagaggcttcggtattggcgctttcccagaactggcccagaggagccagttcagagtgtgaggtcgggtctgcattgaacgtacacaccgaggtctatcagactcccccgattttagcgaagggtgctgactgctgtgctgctagaggctagcaagctccctgtgcgcagctgatgagtttcagcaactcgccacctgggcgcttttctttaaattttgggagtaaactgggaaaataaaaaaatctccacgtccactggctctctccccttctccaacttcctctttcgactcgtttgtgggagttttctcctctttgctgggactataatgtgatgcgcaatcgtttgtgaatgaacaaaagtcaccggcaagcagggagacggggacagatcgctgacggcagattgagggtggcagcaaaggcccggcctccaaggataatggggagccgttttccctcacgcctggtctctatggcccccttcgtcttccaggtaaaatgaatgttccttcatccatcatccgcagagtaccctcaggcgtgcgtagaatctgctgatgaaacctattagcgccgactgggcagctttgtggagccacccgaggctctccattgtggcctttgtctgcagaatttaagcatttacataatgcattagcacggaactcagcacccggtggggacatcgcgtgccaagcctggcgcggccaacgcttcagcggctccctcacccggcagctccctaggaccaccctcgaggaggcattggagtc

SEQ ID NO: 89 - DNA, 460 bp, synthetic construct
  source 1..460: mol_type = other DNA
cgagacggtgtcggtagcgggggccgcagcgctttgcaaagccggcctctgcccccggcctccttggagggcggtagcgggacctgcgctcgcagcctctccccagggctgtgcgcttcggcctggcggctgccgggcaaaaacctcatttatttccttccttcccgagtgagctcacttcctccccgcgccgctcccttccgtcggccggaacccgctcctgggccgcgcggctgcagccgggagcgcggactcggggtgtcgggcggtgtccgcgcccctccccaccccagggcccttctcgggggagatatccaggagctgaaagagattcctcaggaggaagtggaaacctctgaaaggagaggaaaccgactcttttgcaagaaaactgagcgccggctggaaggcaaggaagccgaggcggccggggagattcacccacatgcctgttgactcg

SEQ ID NO: 90 - DNA, 370 bp, synthetic construct
  source 1..370: mol_type = other DNA
ggcaccccacgattagctgagtgatctagggcaaggacctaggcgtctctaagtcttagtttccccatctgaacaatgtggataatgacacgtacctggggtgggcaggctagagtaaatgggaaaatcaaagcgccagccagagcgggtcgcactgttagttcctgtattttctcctcctgcctctggtttcttccgttccgttctagctgcctgaagacccagcccgacagcgatgcctgtcagcccgcgtcgcccaccagggcagctgctctcccaactcgcatggggggcaccaccccgcctcggtgccctagagctgagcgcagccgcggctccacggggatcgccagggcctcagccctcgcgg

SEQ ID NO: 91 - DNA, 250 bp, synthetic construct
  source 1..250: mol_type = other DNA
tcaaaggtcgggggcgggtagagtgggggtggggggaaccgcgggacgccctagctgtgtgcgtttcgggaggcctcgcagtgccattctgcaagagtacctgctatctcgaaaatcttttgccaccgccgaccgcagcgggagggggcaaggggctctctctgactcagctccggatttgtgcatccccgcagtgcggcacccgcagtcttgacttccccagtctctgactgaacagatgtttttgtcc

SEQ ID NO: 92 - DNA, 815 bp, synthetic construct
  source 1..815: mol_type = other DNA
tggcattgaagctgcagcgcgctcacctgtactggtcgaacttggcgtacattgcccactcggtggggtcgctctcgtaggcttccatgatggcctgcacctcctctacattgacctcatcgccggcaaagagctggtgcaggatgcggatcagatcagccagggtccgtggcttcagcacttcggtctgttccatctcgtggggagctggctgcgcgcgcgtctcactgctgggctgcggtggaggagctgagcgagccaaggagctgggggcgagggagcctaacagcccgctagaccgctaagcagacacacacgcacaaacccagcattagagtgccgaaacgtaaggatgtcgtcgcagagacagcaagagacccacccccaggcccctggcagcgcagtggatccgggatcgctggagacgcggtgcacacacaaatcaggttcagatctgtggggttcatcctcccgggccccttttaagcgcttggagtcactaggaatgtaccaacggccctcggagggaggacgaggcggagagccacccaagaaaggtggcggaggcggggagaccctgcgggcacggctcacgcgcacatccccggcttccccgggctccgcgccttcccaagagccccgttgtctccggcgtcccagggatcgcgtgggctccgcgcaatctctcccccactttaacggcgcgttttagccgcccggcctaacgcctctccccgctccacctccgccgctgtggttcgcgacgctgggacgtagacaaaaagggtcggaggagatggctgcggggactgacg

SEQ ID NO: 93 - DNA, 300 bp, synthetic construct
  source 1..300: mol_type = other DNA
tctcccgtcctgtctgtgtgcgtctgttcgcgtgtctctcactcagcccctcttagcttccctccagcctcgcgggcgcgaaactctggcccccatgtgccggagcgagggggcccgcgtccccgggcgcgcgcagagtcgccttcccctctttcctctcttccgcagagcggcgggagcccccgcggtaaccccgccccggcgggcagacggagccagcgaggaccccggcggacaaattagtctccccttcgatcgctcttttccgatcaagtcaggtttcccctgttcccccgacgg

SEQ ID NO: 94 - DNA, 260 bp, synthetic construct
  source 1..260: mol_type = other DNA
gtccccgacgccgaagttcacgaagttgttgttggcggcggcagtggccggctgcgctggggaaggcccggcggggtaagcggcagtgcagctgtagccagggctgcaggccgcgccgccgtaacccggataggcggggtaggcgttataaccgtagggattgaggcccacgccgtaggcaggcgcgtagggcgccgagtcccctaggcatggcttgccatcgcgcaccagcactggcaccgcgatcctgcgggcaggcg

SEQ ID NO: 95 - DNA, 220 bp, synthetic construct
  source 1..220: mol_type = other DNA
cgggcgcctcgggaggcctcttaccggcgggcagcgggaaggcggacgcggtgtggagcagcaccaggcagacagccacgacatcccataacttcatcttaaagtcccgtccggcggcggcacctgcgcgggcaggcgggaggtgggggagagaaccgcagaatgcacgttaagcctgggctccctgcgggtccctgcgcccctctccaacctcgtcacc

SEQ ID NO: 96 - DNA, 310 bp, synthetic construct
  source 1..310: mol_type = other DNA
cggtctccgcttcggtggtttcaggtcccgggttctgtctggcggctctgcttctaatgcggagctggcggttttgcagcaacgcttttgccagtagcgcccactgagcggtttttcagttgctgcaccgttcttagcgcccaacggaacgtttcccgtacgcggagtccataagttgtctgtgtcgtcactttctcggttctggcaccgctagtatctcctaggaacttcctaaacccgttttacggtgcagaacggtaattgcaccgagcctctgtgccaactagtacttccgtaataccgttagccg

SEQ ID NO: 97 - DNA, 293 bp, synthetic construct
  source 1..293: mol_type = other DNA
cgttatggcgcctaaacggatatttgtttggcaataccagcgctatccgctaggtgccggcgcttgctgagtctcagcgccgccagattcttgctagcgaggtccagagcagtgcagagacctagcggacagttttccggtggcagcaatgctcatttcccggacacagatggtttttggcccctgaagaactctttaatccctgcgttttgtagcgctcgttctctgtaaggtttgcttagtatccgtttcatttcgacttcttttctcgctgtttttctgtcggaattacg

SEQ ID NO: 98 - DNA, 490 bp, synthetic construct
  source 1..490: mol_type = other DNA
gggcttgccgcctccttccatgcccggaggacgcggccggcgacggcgcgggctccagactcgccggccgcccggcgccggagacacgtagccgagagggtaggggcccgagccggctgccggcgagcccagctgcccgtcgcggcggcggcggcggcggcggcggctgctgcttcccgaccgcgccgctgctagctgcgcgcctggctcccgccgccgccgctgcccttagtggctgcggtccgggctccggtgcggctggtgctgaagctgaggctgccggagctaggcgaggctcagctcggctcagccctcgcgccccagcgcctcccctcggctgccctctgttggccagaaagggtctctccccgtccgctctccacacatgctcgttactcttgctctacttctgcccagcgtgacattgaactcaagagccctcggaggagacattcattggtacacgtggagaaggactcggtgtatgttt

SEQ ID NO: 99 - DNA, 260 bp, synthetic construct
  source 1..260: mol_type = other DNA
ggcttgtgtgccctagagggcgctgggccccgcgcgctttcctggctgtccccgccggctttccaccctccccaaagcccaggtgcccaccgtgggtcgctgcggcctttccccttcttggccaaatccgattacttcgcagcctgcagatggcatcgccggctaagggcagcctgcggcaggtccccgagcctgagcactcctcctatctggggcctgagaggacgctctgggctttttcccaggcccagggtgcgcgg

SEQ ID NO: 100 - DNA, 320 bp, synthetic construct
  source 1..320: mol_type = other DNA
cccttcctgtcgccccctctcctggtcttcccctgtcaccccattctccgggagaggtgggagggccgcctgagcctgggagctggagtcctccaagcctggaccaagccggaaggagggggccgtgaacttccttgggtcacgaggggctggaatggaggtgggggatgggggcgaagctgagggttcccggggctactgcggggtgtctcgtgctgcgcagggggctgcggccctggggcagacgacccaggtgctgagcgagacgagagcctgggcagggggaagcttcactgggggccagaacaggcgttctcccc

SEQ ID NO: 101 - DNA, 420 bp, synthetic construct
  source 1..420: mol_type = other DNA
aagcagcttctcggaggcggcgtcggggcgccctgaggtccctagtcggcctctgggcgccgagcacgttttatttatccccacagaggatgctggaggacaaaagaaatatttggacgtcagagctgatttatatttttggaagagctacgtttccttttctgaagagaagcgagcgcgccccatatcctgagctcggcggggaggacgagaggggagttttgatgcgccaggaagttcatctatcgcccaggcgctcaggacccggcgccccgccgagttcgccgccctcggaaatatttatttatttgggggaagggagggcagcggggaggagaccagccagacgggagaagaaagccctgcccgggctgggttgggacctccagggcgcttccccgcgcggccgcagccggccag

SEQ ID NO: 102 - DNA, 410 bp, synthetic construct
  source 1..410: mol_type = other DNA
gaatccggcccgaagcgacgtgcactttatggtccccacaccactcggttaactaagaaaagacccgggcgaatggacctaacgcagcccggtgccgagagggcccggtccagcagcctctggggcccagtgcgcagggcactgcgggccgattgcgccccggggccaggaggcgccgagaaagcaaaagcaaaagccggcggcgggtggaggtcaagcggcccggtgcaggccggggaccggggagccgccccgggagagcagggtccaggccttctctgtccccctccagcctggggctacctcagggactcctcgcgctgccagatgcttcggaccaaagggcatttctctgcgccctgaagtcccggcttctctagaggaagtctaagacgttcgggacccacgaa

SEQ ID NO: 103 - DNA, 573 bp, synthetic construct
  source 1..573: mol_type = other DNA
tcgtgatcccccccgaacatcgccgtccccccgagtaacgcggtctccccgaacatcgcggtccccccgaacatcgcggtacccccgaacatcgccgtctccccgtacattgcgatcccccgaaacattgcgatctccccgaacatcgcgatctcgccgaacatgcccggctgaaggcactcagttcccctccgcggctcctttcctccgggtccgcttcctgcggctgctgcttgccccataggccaggaggcttctgggtggaccagcgcgatctccccgaatatcgcggtctacccgaacatcgcggcctccccgaacatcgcggtctccccgaacatcgcgatcccccagaacatcgcggcctccccgaacatcgcggtctccccgaacatcgcgatcccccagaacatcgcggtctacccgaacatcgcggcctccccgaacatcgcggtctccccgaacatcgcgatcccccagaacatcgcggtctccccgaacatcgctgtctccccgaacgtgcctggctgaaggcactcagttcccctccggggctcctttccgccgagtccg

SEQ ID NO: 104 - DNA, 500 bp, synthetic construct
  source 1..500: mol_type = other DNA
tgccggggaaaagtggcacaacaggaaagcctcgccagagggcccttcccagtcggcttcctcgccataaaaagccagactccagccgaggtcctgaccccttccaaatatacgttcccttgcccgcggcccgccactcccgcccgggcctcgggctgtgcagcttcctcctaggcctgcccgggcccatgcctcccccgaagcggcgggagtcggtgcagcctcaacttctactgcgtgtcccaggtcgccaaccagctttgctcgcgtgccgttggcccggaggctgccgcgccagtctctgcgtgcgcctgagcgcgctggagcatttgcgaggccagggctgccgacggcgagaggatactttgaggaacacggtgcttcagtcccgcccccgctctccgcccgagctggaggttggggcgcgctgagccgcagcgcctggacaagtgcgcgggccggagccgcccaggcctgcacccgggggctcgcggccgacg

SEQ ID NO: 105 - DNA, 254 bp, synthetic construct
  source 1..254: mol_type = other DNA
cgccaagcgccatcgcaaagtgctgcgtgacaacatacagggcatcacgaagcccgccatccgtcgcttggcccgacgcggcggcgtgaaacgcatttcgggcctcatttatgaggagacccgcggtgttcttaaggtgttcctggagaatgtgatacgggacgccgtaacctacacggagcacgccaagcgtaagacagtcactgcaatggatgttgtctacgcgctcaagcgccagggacgcactctgtacg

SEQ ID NO: 106 - DNA, 220 bp, synthetic construct
  source 1..220: mol_type = other DNA
ggttccttaacaaacagcgggcttcccggtagccgggttcccagcccaagaaccttctcattcacctgcgcagcgccgcgggacctcacccttccggtgtggtgccaagatccagactgcctgacttgttttcgtgccgccggacggtgtgtttaatcgatgctgatgttttactctctagtttgaggacccagcgacagggctttagcccctcttcctg

SEQ ID NO: 107 - DNA, 500 bp, synthetic construct
  source 1..500: mol_type = other DNA
caggagcgccgcgctgtctggggactgtggggcagtggtccctcggggcggacagagcttgggtacagggggctcgcgctccctgatcccggcttcaaggttcagcttgaagagagtcggtttacctttccacccacaccgggccctggtgcgggtcgaaggccacaggctgagaatttgctgggaggggtcgggaagaagcgtaggagcgctgaccgcctccccttcctctttctctgtctttgcgggcggcgctacttccctttcccgaattccgagcccccctctccaggtcaaatgctcggcgcggcctttccgagactacccgagaaattctgacggtgagcgccagacagcggcgtggtagcggccttccagggaactgcctctcgggagaggaggggacgggcggggagaggcccttggcccctctcattccctgagtccctgcggcggcgcccctcttggagcttgagacctcagggattgaggatcgcggg

SEQ ID NO: 108 - DNA, 210 bp, synthetic construct
  source 1..210: mol_type = other DNA
tttcagtcctcgggctcggtgggtgcatcctttattggggagcgcgggggctgcccgtgcccctccagtcgctccccgtagggagctgcgcactgcggagccctcgcccccgagcgagcctgtctcggactcccaaccgtcagcctcgttccgggccgcggaggccggagcagctcccccggggcagcgcaaccgctggggccggcctca

SEQ ID NO: 109 - DNA, 640 bp, synthetic construct
  source 1..640: mol_type = other DNA
cacacttagcctgacctctgggatctagcgggcaggagagagacagacgagaggagctggtggggaaagagaagggaggtcaggagggaggtcaggaaggcgccaacagcgccctctcccggtaagtgggcctccctcccgcgttctacctgcaaggccgaagggagaaaaccaaatgttttctcttgacggatggccgggactccttggccctcgcctggctttccacccctcctggcttcccgcaccagccgggcccgcagctcacctgccggcagctggggcgaagccgtagtcggcgctgccgggcgctttgtgcttggcctccgcggcgccccgggcggcgccctccagggacagcctcggcgcgtgcaggcctccggggggcgcgcgacccgccgagttcacgcgccgcatctcggggcctccgggctgcggcccgaagcagttgggagagctcaggctgcggccggtgccaccgtggggtagccctgggcctcggtgcggctccccgacgtacaggcgcttctttatgagcgagcggccccctcccgagaagcgctccaggcccccagccccggcgtagcgcgcgcccgcgggaaagcgcgagaagccgagagccgggggcgccccggggcca

SEQ ID NO: 110 - DNA, 800 bp, synthetic construct
  source 1..800: mol_type = other DNA
gaggaggaggaggaaacacaggccgcacgaaggtccaagccccaagcccccaagcccccggcctctggacggccaaaccccgaggcgcgggactggaaggacaggtaccaggctgcgggcgcgcggctgtggccatctctttccgccctgaggccgacgaacccggctggaagctgagtgcctagcggcccaaagcagcccgggcgccgggagggcgccagagaagcacagcgttagggcggggaagaaagggtgaatctcagaatcgaaatccgcactggcgcccacgaccctgggcgccggcctggtcctcggcagctttctggcggctgcgcttgtgtgtgaatgtgtcccgggaggaccggacacctcaatcccccggcccccaacgcgggcgcctgtccgcgagcgccgggccagacgccgaagaggaaggtgaccgaacccgtagcagcttccgagagcgtacccgtttgcaaattgctgcaggaagagcgaggcgggccttgcgctttttaatccggaacgggaagcactggggaagggaccgaggttaacttcgacctccgctggggcagatacgtaaaccttctcaaacctccgagttcaccttacagcgaacattaaatacccttgcgactacaatactaacaaatcgaacactgacgtaaaatcttaagaataaacgaatctttgttctgatagagctcgttcaaaatggaaatacactccacacatcttcctctataacgcatgtcacaaatacagctttgcagagaagaaacgaaataaaataaagt

SEQ ID NO: 111 - DNA, 400 bp, synthetic construct
  source 1..400: mol_type = other DNA
cggccatgagtttcttcttttccagcagggcgtcggagactcgcactttgacttggttcacatacacaggagatccggattcgctgggcacgtcgggaacccgcgagttaatagaggtgaccgaggacgtggacccgggggagtcggttatcagctgggctcgggtcaagcgcttgccggtcctgttgggcgtctgtttcaaaatccgggagcgggcttctacgtagatgcggccatagagggcgatgaggagcagggtggggaagtagaaagcacccaccgtggagtagaccgtgtagaggatgtggtcggtgttcaccacgcattccgacacctcctcttcggccttagcctgacgccagaagaagggcggcagcgagatagagatggagaagaccca

SEQ ID NO: 112 - DNA, 330 bp, synthetic construct
  source 1..330: mol_type = other DNA
cgcacgaaatccggggcgggcactatgtgattcgagcccgctgcgccctgggctcctcgtgtcgcccgcggggaaaggaggagggagactgctcacatggcccacttcgcggcctgagatggagtgcgcgcagggcgtcagggcagagggaggcggggattcaggataaagagcggcctgatgcccagagccgggtgcccggacctcctgcctcctgcttctgatccctgcaagccgcccaccctgcgatcgccgggtcccgccctgacaatgccctgccgccagttccagccccagtgcagccgtcgccggtcctgtgcgcctctggtg

SEQ ID NO: 113 - DNA, 250 bp, synthetic construct
  source 1..250: mol_type = other DNA
cttctcggacgcggggaaaggggagacaggaggcttccccttgcgcggggtgggtcggttcttctcacacgcactcgcgcctcccccaacaccaaccaaaattaattttaaaagaaccagagttcaagtttcagccccctgggtctccctctcgctgtttcttgaagagcaggtgaggcgcccttgcttaaaagggaagcgcccaggaccggagccgcggccgaatccctccagccccggcctgggcgag

SEQ ID NO: 114 - DNA, 310 bp, synthetic construct
  source 1..310: mol_type = other DNA
gcgccgcacctgctgggccgcggggctcctactggggcgcgggctggtggctgggccgcgggggcggcgagtcgtcctccgaggagcagtcggaggaggcggcgtggacgctggcgccgttgctgtaggggaaatggtcctcgtcgtcctcgtcctcgtcctcctcggggtcactgtccctcaagtcccgcaggcggcgtccgctgcccttgtctccgggcgctggcggccccagcagctcctcggctcccggctcctccgcgccgcccttccccgcgcccccgccgccgcccttctgtttctccgcttc

SEQ ID NO: 115 - DNA, 101 bp, synthetic construct
  source 1..101: mol_type = other DNA
ggccctgccaccccaaccccggccgcagtggctggaagccgtggccttttccgggctccgagagccggcggggcggttttcctccgggtttcctcccgccg

SEQ ID NO: 116 - DNA, 450 bp, synthetic construct
  source 1..450: mol_type = other DNA
cgcgctcggagtgcgctgcctcgctcacgcggtcccaggccccgcttcttcaggcagtgcctggggcgggagggttggggtgtgggtggctccctaagtcgacactcgtgcggctgcggttccagccccctccccccgccactcaggggcgggaagtggcgggtgggagtcacccaagcgtgactgcccgaggcccctcctgccgcggcgaggaagctccataaaagccctgtcgcgacccgctctctgcaccccatccgctggctctcacccctcggagacgctcgcccgacagcatagtacttgccgcccagccacgcccgcgcgccagccaccgtgagtgctacgacccgtctgtctaggggtgggagcgaacggggcgcccgcgaacttgctagagacgcagcctcccgctctgtggagccctggggccctgggatgatcgcgctc

SEQ ID NO: 117 - DNA, 1910 bp, synthetic construct
  source 1..1910: mol_type = other DNA
ctgtcatggggacccttgtggcccgggtcgcccgcagcctcatcttggcaggatttacgccgccactggccgaaggcaagaagtggaaggaatcggccgtctcccccagcgtcccagctccggctgccctggctgccgccgctcacggacaatctagttgtacaaaaggctctctgggctgcactgctttcgaagaacggcccaaagtatctcggtcctgggcctgggcagccaaggagaggggcggccagtcttggctcgtcccgaagtgcccgccccgccccctctcgctgcagcagccgcctcctctcccgtagccctgcgggccgctcttcactgctctccagacttggggccctatctgaggcgtcccaaacaccaacttctggctcctggccccaactcgagaggcttccagcgaggacgaaggcaggctcgagagaaacctggcgggccagcagatccgggaggccggcgtggaggcggcggcggatttgaagggaggagacacttactgggatcgatggggggcttgtctccgccgctctcattctcagcattgttttcagagaaggcgccttcgctgggttgtttttctctatcaactggaggagaaccacaagcatagtcagtcagggacaaagtgtgagtgtcaagcgtgggacagtcaccccttctggccgacagcggttcaggtttaatgccataaggccggctggagggcaagcccgcgaaggagagcgcaccgggcgtgggctccagccaggagcgcatgtacctgccgtccggcgccgccgccgccacgggcgcctgggggtgcacgtaggggtggtggtgatggtggtggtacaccgcagcgggtacagcgttggcgcccgccgcgtgcactgggttccacgaggcgccaaacaccgtcgccttggactggaagctgcacgggctgaagtcggggtgctcggccagcgtcgccgcctgccggggaggctggcccagggtccccggcgcatagcggccaacgctcagctcatccgcggcgtcggcgcccagcaggaacgagtccacgtagtagttgcccagggccccagtggtggccatcaccgtgcccagcgcctggcccgcccggcccgacccacggaaattatgaaactgcagatttcatgtaacaacttggtggcaccgggggggaagtacagtcacctaataagttgccggcgcccgcgcccccattggccgtgcgcgtcacgtgcccgtccagcagaacaataacgcgtaaatcactccgcacgctattaatggtccgatgttttgcagtcataatttttatagcaaaagccatatgtttttatgtaaagggatcgtgccgctctacgatggggtttgttttaattgtggccaacgacgattaaaagatcaaatctagccttgtctctgtactctcccgtctccccccccatacacacacttcttaagcggactattttatatcacaattaatcacgccatcaagaaggcgcgggtcccgcgtgcgagtgcggccagcggagcccctcacataaaattagacaataattgaagccataaaaaagcagccaaatcgcattgtcgctctactgtatttaaatctatatttatgatatttcataaggagttattgtttcagaagccacacaggctggcgggaagtcggaaacgaccaacagattcgtttgcctcgccgtggctcccagctgtaaaaatttacgaggacttggaaaggttagactgttgtgtttggttggcgagctccctgtaaataatccctgcggtccccgggagaggcgagtttacccgcggccgccctcgaaaagtcaaattcaacgcaggatccgtcccaaacggagccgccgccggccctaccagggcactccagg

SEQ ID NO: 118 - DNA, 270 bp, synthetic construct
  source 1..270: mol_type = other DNA
tcaggagcgaagctgttccagctgacccgcgcgtctgggggcctatgcccggcttccgattccatttaaaacgacccgcgcatcttatctccgtcgcctccccggggttcccacccacccccctccggcccgggccaggccagcccagccccggcggaagccaagctgggagcttttgaagtccggagaatttcaatccgagaggagccggctggaccggagcccgtcgccccagcgggggaagggacggggggcctgccgtgtggcagg

SEQ ID NO: 119 - DNA, 420 bp, synthetic construct
  source 1..420: mol_type = other DNA
tacgggtatggccaattttgtttgaaagctcgctctttctctgcgctttcctgtcgagttcccttcaggttctggtttcctccgacggtggcaagggatgtttttcacgcctttcaccacttttgcccgcgcctgcgtcccctgcccgaggtcccaggccgcggcggcggcgcggtgggttcattagcgggcgcgccgggcccgcgcagcgctgtctgcagccgcgtcgccgcatccatcatcattacatcagcgcggggaagtccacgccgagttctcccggccccggcgcgccccagtcagcgcgccagcaaacagaacatgctggattgctcagagacggctctgggtgtctgggggcagaaggggtgctcccaggcccggctttgatccgcatttctgaagctggctttgggagac

SEQ ID NO: 120 - DNA, 490 bp, synthetic construct
  source 1..490: mol_type = other DNA
cgaagaacgtttctcctacctagagcgcagccgttttgcggaaggcacagtccgggctccgggagccaagcagggatccgggcctcgttgcggaggggacgcccgggctgagctgagctcacttgctggcggcggcagcccctgcagtcatggggtcaccggcttgtggagaggggctggcgctgttccttggccccgctccccgcccgcaaagggccgggaggtcgggctcccggcagtgtccaggctctcgcccgccggtgcctgagccgtacccaccgcgtcccctccgccccaacagctggtgctgagggtccggcccggggtcatctggcagggccgtgcggctcctgcgggtgaccaggagcgcggagccccttcccactcgggctcgggtggcctcacctttcctggggctgcgtccccgcgggacgccaggttcccggtttcttctaggtcgccctggttcctggcagctcctggtgccacg

SEQ ID NO: 121 - DNA, 350 bp, synthetic construct
  source 1..350: mol_type = other DNA
atgctgccctcgcccgccgccgccggtgctgatgcggttgctatggttatggaaccccgcaactgcccccctctccgtgcaacagctgcccggccgcggcctccgggcagccccagcgagcaggcgccaggacgcgaggctgtcagcgcggtcgggggaacgcagccgccagcgccgcaagaaagcaaggaccgaccggctgcgcccgcgccgctgggctggcgcctcccgggctgcgctcacctctggcgccctcactcctctccttcctccgccgggacgcggccagaccctccgcgccgcgccgcgcgccctcgcggccgggagagcccgccctgctgctcccgctg

SEQ ID NO: 122 - DNA, 350 bp, synthetic construct
  source 1..350: mol_type = other DNA
ttctcaggtactgaaagttttccgggctcttccgcacccgcggatgtggcgaagccgcggggcagctccgctcgcgctccagtcgcaggatgtccttgaccgagaagggggtggaggtgacggggctcagcagcatcccgaaggcggatggggcggggccgaggaggtccgggtgaggagcggcaccctgaacttcccgtcttgtcgctgcaggccccgcagacagacccaagctctgggacagacgcccagcgtcccagacagcgccttcctctgggccatgctggtaggcccgggtccagggccgggtgacgagaccgtagccccccattggttctcgcagaaaccac

SEQ ID NO: 123 - DNA, 420 bp, synthetic construct
  source 1..420: mol_type = other DNA
gcggagcgcgccgtcgctgccagtgtccccctttaacattccttccccgagcctttccctgctcctccgctctccgagcccggggcggccccgcagacaccagcgcctctatcctgcgctccttggcgtacgcagccgggcgaggtcgcggcgggaatgggcgccgaggccctgcccgccagggccgcatctgcggcccgagacttacttctgctttgctttcctgcacctggcccggggcaggctttctagtttctgcctcggccagaggagccgacttccgcaggacgggcttggggcagggggtgccggtgggggcagcggggcgaggcgaggtcgcagggagacgaccaggcctgcttgggcctgggccgctccagccgcctcgcctcttcagggacctcgccccgggaccacccg

SEQ ID NO: 124 - DNA, 290 bp, synthetic construct
  source 1..290: mol_type = other DNA
acttccctggggacgtggggaattgggtaccacggtggcagcttgctgtaccagggcagatcgtagagaggctccctctgtaggcccagacccgggctcagcaagggaggcgccatccggaaggggaaggggggcatggcgggggcagcagacgctgcggcctccagctgccggaaaggttggaagtcttcctccacggtggccaggctgtttctgtagtgtccataggacgggaaaggcgtgtagtacgcggccaggttctgcgggctgctctccggcgggtagcacac

SEQ ID NO: 125 - DNA, 270 bp, synthetic construct
  source 1..270: mol_type = other DNA
aaagaccgcatttgaattctcctcgccggccattgagggagagaaaggaacgcttagttccattcacattcacagaaagaagcgccgagggtgggggaaacgcagtcttgccgggtgagccgggacaggttcctcgcctgccccccggccgctgcttcctcttagctgaatggggagcgacccgccccgggcgcggccttcggggctgaagactgaggtgcagcctcacccccggcctggcagcggcttggaagagagagggaaaggagg

SEQ ID NO: 126 - DNA, 200 bp, synthetic construct
  source 1..200: mol_type = other DNA
cccaccgagggaggttagaggaaggcgctgtggctgtgggattaggaggccacagacgggaaccgcgccgagctagactagctgtagaaggctgtctctccaccccgccacagcgctccgagaaggagcgggaggcctgggcaggggagaggcgctcctgcccccacccgcgccctgctggccacctgtggcgtcgggga

SEQ ID NO: 127 - DNA, 737 bp, synthetic construct
  source 1..737: mol_type = other DNA
ggttcttgcgctgggatctggtgaataccaccagcagggcccgctcctggggaggccgcaccctccggccgaagcccagactccgcaggtccgggggcggcggttgctggggtccccgcgcgcgcgcctcggcctccccggcgtccagctcgccccatgcggcccgcagctccaagcacagctgcttccagggctggtggcgcaggccctgccacacgtcgaagacttcccagccggccggcggcgccccctgcgggtccagggtccgcgcgtccagcagtaggggcgaaaggcaagggaagagctgcacgtggagcggcccggctggtggcccccagggcgctgagggcgcctggcgaaagagccgcagctccgcgcccaccagctcttctttgtctgagagcatggacacatcaaacaaatacttctgtctccggagaggagtgtgcgagagatcgtctgcgagataaaaaataattacagtcagtttcacttaagggggagatcagcccggtgctcttcggccgccccgggaggaaaagggcggggagtgggggcaggtcggccgggcagtccagcttgcccggcccagggcctgaccaccccggctccccatctggctggtgcatggcgcggggaagggggcgcgccaggacgggccccctccttcgcgtcagctccggactctcagggcggaagtcggtggctgctggcgaggcggcggcggcggcctaccg

SEQ ID NO: 128 - DNA, 560 bp, synthetic construct
  source 1..560: mol_type = other DNA
tgtggatctcggtcctgctagtctggaggccttggctcgggaacagaggcctccccagactgggtgcacatgcaatccctcctctatctccaggatcgggggcatacagcgcagtggcggggatgggggtggggaggctacagggcaaaccagggtaaagtgatttcgcgtctacgccggattggggcccccaagacttttccccaacccttgtctgaatgccacaggcttccggagacacctccgaacagcccgcagggtcttttggtcctgtgccaaattcacagggcgcagcgaaccagttatggtgcccacttgggggaggggcagagattcttgccacttcattcccgggcaaagaaaatccgcgcccaaccgccggccagcctaggcctctgggacttcttctcttgaggaagggcctcttgagcagtcttctctgagcttccttagcctccttccgcaattgtagtagagaaagtgcacgattaacagaggactttgagggacagaaacaactgtagcaaggcgtgcgtcgtggggagggttggcgagctgca

SEQ ID NO: 129 - DNA, 370 bp, synthetic construct
  source 1..370: mol_type = other DNA
acaccgagacggtaggcgcgcggctgtggggtcggggctgagagctgggatggggccgggccagtcagcgcctctgctccccgaagtttggggagcgtccttcgtgccgcacgggactgggtgctggggatcctcggtcagaatgcaaggccggtggctcccggttcgggggaaacccggctgctgggacgcagaagggaaacaaggttgaaaccgaaatctcggccctgggggtagaggagagcgtttcttccgaactggaagcgaagtcccatccgcggcccggggcggctcccttctcaccttgcccggtgccggggtcgacagccccgcgctctcctccacctctcggctccggttgctggcggcg

SEQ ID NO: 130 - DNA, 450 bp, synthetic construct
  source 1..450: mol_type = other DNA
gtttttatcctgaagaggtggagaattcctggaggaggctccggaaggggggagggtcggtgggagagtccaaaaatctgtcacaaaatggagtctaatcgcttgtaagacagctcccaggtttggcgacccgagacccgctggggtgcaagcgggcgcctttgtgcacggcgagacgcggggttccggcccgggccgcgctcctacctcggcggcggggccgcggcgtccttccgcccgcagggcctgcccgggcggccgagcctctcggcgacacagacttcagggctccgccgggcccgctgggggccctttgggctcgcctgtccaggtaggatgtgcctacgttggcagcaggggagcatcgaggctttgattgagtttcacagaaagccctcttgagtcctggggtgagtttttaaaaaatttcccccagctggctgtggctcc